(19) United States
(12) Patent Application Publication
(10) Pub. No.: US 2026/0174765 A1
(43) Pub. Date: Jun. 25, 2026

(54) Methods for Modulating Complement Factor B Expression

(71) Applicant: Ionis Pharmaceuticals, Inc., Carlsbad, CA (US)

(72) Inventors: Daniel A. Norris, San Marcos, CA (US); Steven G. Hughes, Carlsbad, CA (US); Michael L. McCaleb, Carlsbad, CA (US)

(21) Appl. No.: 19/126,841

(22) PCT Filed: Nov. 2, 2023
(86) PCT No.: PCT/US2023/078495

§ 371 Date: May 2, 2025

Related U.S. Application Data
(60) Provisional application No. 63/382,057, filed on Nov. 2, 2022.

(51) Int. Cl. A61K 31/519; A61K 9/00; A61K 9/48; A61K 31/19; A61K 31/353; A61K 31/357; A61K 31/501; A61K 31/554; A61K 31/70

(52) U.S. Cl. CPC A61K 31/519; A61K 9/0019; A61K 9/0048; A61K 9/0051; A61K 9/0053; A61K 9/4858; A61K 31/19; A61K 31/353; A61K 31/357; A61K 31/501; A61K 31/554; A61K 31/70

(57) ABSTRACT

Provided herein are methods of ameliorating a disease or disorder associated with dysregulation of the alternative complement pathway by administering a Complement Factor B (CFB) specific inhibitor to a subject.

### RELATED APPLICATION

This application claims priority to U.S. provisional patent application No. U.S. 63/382,057, filed Nov. 2, 2022, which is incorporated by reference herein in its entirety.

### REFERENCE TO AN ELECTRONIC SEQUENCE LISTING

The instant application contains an electronic Sequence Listing that has been submitted electronically and is hereby incorporated by reference in its entirety. The sequence listing was created on Sep. 29, 2023, is named “BIOL0467USLSEQ.xml” and is 45,762 bytes in size.

### FIELD

Provided herein are methods of ameliorating a disease or disorder associated with dysregulation of the alternative complement pathway by administering a Complement Factor B (CFB) specific inhibitor to a subject.

### BACKGROUND

The complement system is part of the host innate immune system involved in lysing foreign cells, enhancing phagocytosis of antigens, clumping antigen-bearing agents, and attracting macrophages and neutrophils. The complement system is divided into three initiation pathways—the classical, lectin, and alternative pathways—that converge at component C3 to generate an enzyme complex known as C3 convertase, which cleaves C3 into C3a and C3b. C3b associates with C3 convertase mediated by CFB and results in generation of C5 convertase, which cleaves C5 into C5a and C5b, which initiates the membrane attack pathway resulting in the formation of the membrane attack complex (MAC) comprising components C5b, C6, C7, C8, and C9. The membrane-attack complex (MAC) forms transmembrane channels and disrupts the phospholipid bilayer of target cells, leading to cell lysis.

In the homeostatic state, the alternative pathway is continuously activated at a low “tickover” level as a result of activation of the alternative pathway by spontaneous hydrolysis of C3 and the production of C3b, which generates C5 convertase. Dysregulation of the alternative complement pathway is associated with diseases and disorders, including kidney and ocular diseases and disorders.

### BRIEF DESCRIPTION OF THE DRAWINGS

FIG. 1A, FIG. 1B, and FIG. 1C show plasma CFB protein levels following either single or repetitive administration by subcutaneous injection of Compound 696844 or placebo in healthy subjects. Dates of sample blood collection are indicated by visit day on the x-axis, and dates of administration of Compound 696844 are indicated by arrows. FIG. 1A shows mean (+/−SEM) percent change from baseline of the level of plasma CFB (mcg/mL) following a single administration of placebo; or 10, 20, or 40 mg of Compound 696844 (N=6 per group). FIG. 1B shows the mean CFB protein level (mcg/mL) following repetitive administration (8 times within 6 weeks) of placebo; or 10 or 20 mg of Compound 696844 (N=6, 12, or 12 per group, respectively). LLNR, Lower Limit of Normal Range=146 mcg/mL. FIG. 1C shows the mean (+/−SEM) percent change from baseline of the level of plasma CFB (mcg/mL) following repetitive administration. Baseline (BL) is defined as the average of the measurements on Day-1 and Day 1 (pre-dose) including unscheduled measurements between Day-1 and Day 1 (predose).

FIG. 2A, FIG. 2B, and FIG. 2C show systemic levels of various components of the complement cascade (CFB protein level, CH50 activity, AH50 activity, and Factor B split product (Bb)) of subjects receiving subcutaneous injections of either placebo or Compound 696844. Dates of sample blood collection are indicated by visit day on the x-axis, and dates of administration by subcutaneous injection are indicated by an arrow. FIG. 2A (placebo) and FIG. 2B (20 mg Compound 696844) show the mean (+/−SEM) percent changes from baseline of the levels of plasma CFB protein (mcg/mL), plasma Bb (split product of CFB) (mcg/mL), serum AH50 (a biomarker for Alternative Complement Pathway activity) (U/mL) and serum CH50 (a biomarker for Classical Complement Pathway activity) (U/mL) is indicated on the y-axis; baseline (BL) is defined as in FIGS. 1A and 1B. FIG. 2C is a scatter plot of serum AH50 hemolytic activity (UmL) (x-axis) and CFB protein level (mcg/ml) (y-axis) in subjects receiving placebo or prior to administration of Compound 696844 (Group 1, pentagons) or after administration of Compound 696844 (Group 2, diamonds).

FIG. 3A and FIG. 3B show the urine protein/creatinine ratio in 24 hour urine samples for individual subjects prior to administration of Compound 696844 (baseline) and at week 29. FIG. 3A shows the urine protein/creatinine ratio for each subject and FIG. 3B shows the percent change in urine protein/creatinine ratio for each subject from baseline to week 29.

FIG. 4A and FIG. 4B show the urine protein excretion rate in 24 urine hour samples (mcg/day) for individual subjects prior to administration of Compound 696844 (baseline) and at week 29. FIG. 4A shows the urine protein excretion rate for each subject and FIG. 4B shows the percent change of the urine protein excretion rate for each subject from baseline to week 29.

FIG. 5 shows the glomerular filtration rate estimated based on serum creatinine concentration (eGFR) for each subject prior to administration of Compound 696844 (baseline) to week 37. Values were adjusted using CKD-EPI 2009 mL/min/1.72 m². Dates of sample serum collection are indicated by week on the x-axis. Baseline (BL) is defined as in FIGS. 1A and 1B.

FIG. 6 shows the concentration of plasma CFB (mg/dL) for the treated subjects prior to administration of Compound 696844 (baseline) to week 37. Dates of sample blood collection are indicated by week on the x-axis, with the indicated number of subjects providing samples. Upper Limit of Normal (ULN) and Lower Limit of Normal (LLN) are as indicated on the graph. Baseline is defined as the mean of the last 2 measurements obtained prior to the first dose of Study Drug.

FIG. 7 shows the concentration of urinary Ba (Factor B split product) (micrograms/L) for the treated subjects prior to administration of Compound 696844 (baseline) to week 37. Dates of sample collection are indicated by week on the x-axis, with the indicated number of subjects providing samples. Upper Limit of Normal (ULN) and Lower Limit of Normal (LLN) are as indicated on the graph. Baseline (BL) is defined as the mean of the last 2 measurements obtained prior to the first dose of Compound 696844.

FIG. 8 shows the mean (+/−v SEM) percent change from baseline to the average of Weeks 21, 25, and 27 of plasma CFB, plasma Bb, urine Ba, serum AH50 activity, and serum CH50 activity. Baseline (BL) is defined as in FIGS. 1A and 1B.

### SUMMARY

The complement system mediates innate immunity and plays an important role in normal inflammatory response to injury, but its dysregulation may cause severe injury. Dysregulation, such as over-activation of the classical, leptin, or alternative complement pathways can lead to diseases or disorders. Activation of the alternative complement pathway beyond its constitutive “tickover” level can lead to unrestrained hyperactivity and manifest as diseases or disorders of complement dysregulation.

Provided herein are methods for ameliorating diseases or disorders associated with dysregulation of the complement pathway in a subject by administration of a Complement Factor B (CFB) specific inhibitor. In some embodiments, provided are methods for ameliorating diseases or disorders associated with dysregulation of the alternative complement pathway in a subject by administration of a Complement Factor B (CFB) specific inhibitor. Several embodiments provided herein are drawn to a method of inhibiting expression of CFB in a subject having, or at risk of having, a disease or disorder associated with dysregulation of the alternative complement pathway by administering a CFB specific inhibitor to the subject. Certain embodiments provided herein are drawn to a method of reducing or inhibiting accumulation of C3 deposits in the kidney or eye of a subject having, or at risk of having, a disease or disorder associated with dysregulation of the alternative complement pathway, comprising administering a CFB specific inhibitor to the subject. In certain embodiments, the disease or disorder associated with dysregulation of the alternative complement pathway is a kidney disease or disorder. In certain embodiments, the kidney disease or disorder is a nephropathy, in certain embodiments, the nephropathy is IgA Nephropathy (Berger's disease). In certain embodiments, the disease or disorder associated with dysregulation of the alternative complement pathway is an eye disease or disorder. In certain embodiments, the eye disease or disorder is age-related macular degeneration, in certain embodiments the eye disease or disorder is geographic atrophy associated with age-related macular degeneration. In certain embodiments, the CFB specific inhibitor is an antisense compound. In certain embodiments, the CFB specific inhibitor is a modified oligonucleotide. In certain embodiments, the CFB specific inhibitor is a GalNAc-conjugated modified oligonucleotide.

In certain embodiments, methods comprise administering a therapeutically effective amount of Compound 696844. In certain embodiments, the therapeutically effective amount is within the range of about 10 mg to about 100 mg. In certain embodiments, the therapeutically effective amount is within the range of about 40 mg to about 100 mg. In certain embodiments, the therapeutically effective amount is within the range of about 40 mg to about 70 mg. In certain embodiments, the therapeutically effective amount is within the range of about 70 mg to about 100 mg. In certain embodiments, the therapeutically effective amount is about 10 mg, about 20 mg, about 40 mg, about 70 mg, or about 100 mg. In certain embodiments, the therapeutically effective amount is administered once about every 4 weeks. In certain embodiments, the therapeutically effective amount is administered once about every 8 weeks. In certain embodiments, the therapeutically effective amount is administered once about every 12 weeks. In certain embodiments, the therapeutically effective amount is administered once about every 16 weeks. In certain embodiments, the therapeutically effective amount is administered once about every 24 weeks. In certain embodiments, the therapeutically effective amount is administered once about every 6 months. In certain embodiments, the therapeutically effective amount is administered monthly. In certain embodiments, the therapeutically effective amount is administered once every two months. In certain embodiments, the therapeutically effective amount is administered once every three months. In certain embodiments, the therapeutically effective amount is administered quarterly. In certain embodiments, the therapeutically effective amount is administered twice a year (semiannually). In certain embodiments, the therapeutically effective amount is administered annually.

In certain embodiments, methods comprise administering a loading dose of about 10 mg, about 20 mg, about 40 mg, about 70 mg, or about 100 mg of Compound 696844 once about every 2 weeks, and subsequently administering a maintenance dose of about 10 mg, about 20 mg, about 40 mg, about 70 mg, or about 100 mg of Compound 696844 once about every 4 weeks, once about every 8 weeks, once about every 12 weeks, or once about every 24 weeks. In certain embodiments, methods comprise administering a loading dose of about 10 mg, about 20 mg, about 40 mg, about 70 mg, or about 100 mg of Compound 696844 once about every 4 weeks, and subsequently administering a maintenance dose of about 10 mg, about 20 mg, about 40 mg, about 70 mg, or about 100 mg of Compound 696844 once about every 4 weeks, once about every 8 weeks, once about every 12 weeks, or once about every 24 weeks. In some embodiments, methods comprise administering at least 2 loading doses, at least 3 loading doses, at least 4 loading doses, at least 5 loading doses, or at least 6 loading doses.

In certain embodiments, methods comprise administering a loading dose of about 40 to about 100 mg of Compound 696844 once about every 2 weeks, and subsequently administering a maintenance dose of about 40 to about 100 mg of Compound 696844 once about every 4 weeks, once about every 8 weeks, once about every 12 weeks, or once about every 24 weeks. In certain embodiments, methods comprise administering a loading dose of about 40 to about 100 mg of Compound 696844 once about every 4 weeks, and subsequently administering a maintenance dose of about 40 to about 100 mg of Compound 696844 once about every 4 weeks, once about every 8 weeks, once about every 12 weeks, or once about every 24 weeks. In some embodiments, methods comprise administering at least 2 loading doses, at least 3 loading doses, at least 4 loading doses, at least 5 loading doses, or at least 6 loading doses.

In certain embodiments, methods comprise administering a loading dose of about 40 to about 70 mg of Compound 696844 once about every 2 weeks, and subsequently administering a maintenance dose of about 40 to about 70 mg of Compound 696844 once about every 4 weeks, once about every 8 weeks, once about every 12 weeks, or once about every 24 weeks. In certain embodiments, methods comprise administering a loading dose of about 40 to about 70 mg of Compound 696844 once about every 4 weeks, and subsequently administering a maintenance dose of about 40 to about 70 mg of Compound 696844 once about every 4 weeks, once about every 8 weeks, once about every 12 weeks, or once about every 24 weeks. In some embodiments, methods comprise administering at least 2 loading doses, at least 3 loading doses, at least 4 loading doses, at least 5 loading doses, or at least 6 loading doses.

### DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising a 2′-H(H) deoxyribosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside or a nucleoside comprising an unmodified 2′-deoxyribosyl sugar moiety may comprise a modified nucleobase or may comprise an RNA nucleobase (uracil).

As used herein, “2′-MOE” means a 2′-OCH₂CH₂OCH₃ group in place of the 2′-OH group of a ribosyl sugar moiety. A “2′-MOE sugar moiety” means a sugar moiety with a 2′-OCH₂CH₂OCH₃ group in place of the 2′-OH group of a ribosyl sugar moiety. Unless otherwise indicated, a 2′-MOE sugar moiety is in the β-D-ribosyl configuration. “MOE” means O-methoxyethyl.

As used herein, “2′-MOE nucleoside” or “2′-OCH₂CH₂OCH₃ nucleoside” means a nucleoside comprising a 2′-MOE sugar moiety (or 2′-OCH₂CH₂OCH₃ ribosyl sugar moiety).

As used herein, “5-methylcytosine” means a cytosine modified with a methyl group attached to the 5 position. A 5-methylcytosine is a modified nucleobase.

As used herein, “about” means plus or minus 7% of the provided value.

As used herein, “administering” or “administration” means providing a pharmaceutical agent, pharmaceutical composition, or compound to a human subject.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark, or the delayed onset or slowing of progression in the severity or frequency of a symptom or hallmark. The progression or severity of the symptom or hallmark may be determined by subjective or objective measures, which are known to those skilled in the art.

“Antisense compound” means an compound that is capable of undergoing hybridization to a target nucleic acid through hydrogen bonding. Examples of antisense compounds include single-stranded and double-stranded compounds, such as, antisense oligonucleotides, siRNAs, shRNAs, ssRNAs, and occupancy-based compounds. Antisense compounds may comprise a modified oligonucleotide. Antisense compounds may comprise a modified oligonucleotide and a conjugate group.

As used herein, “CFB nucleic acid” or “Factor B nucleic acid” or “Complement Factor B nucleic acid” means any nucleic acid encoding Complement Factor B. For example, in certain embodiments, a Complement Factor B nucleic acid includes a DNA sequence encoding Complement Factor B, an RNA sequence transcribed from DNA encoding Complement Factor B (including genomic DNA comprising introns and exons), and an mRNA sequence encoding Complement Factor B. “Complement Factor B mRNA” means an mRNA encoding a Complement Factor B protein.

As used herein, “Complement Factor B,” “CFB,” “Factor B,” “FB”, “Complement Factor B protein,” “CFB protein,” or “Factor B protein” means the polypeptide expression product of a CFB nucleic acid.

As used herein, “Complement component” means a product, by-product, end product, terminal product, or activation product of a complement pathway. In certain embodiments, a complement component is a by-product. In certain embodiments, a complement component is an end product or terminal product. In certain embodiments, a complement component is a by-product, end product, terminal product, or activation product of the alternative complement pathway. In certain embodiments, a complement component is any of C3, C3a, C3b, C4b, C3dg, C5, C5a, C5b.

C6, C7, C8, and C9.

“CFB specific inhibitor” refers to any agent capable of specifically inhibiting CFB RNA and/or CFB protein expression or activity at the molecular level. For example, CFB specific inhibitors include nucleic acids (including antisense compounds), peptides, antibodies, small molecules, and other agents capable of inhibiting the expression of CFB RNA and/or CFB protein. In certain embodiments, the CFB specific inhibitor is Compound 696844. CFB inhibitor activity, reduction of CFB RNA, or reduction of CFB protein may be assessed by methods described in WO 2015/168635.

As used herein, “dose” means a quantity of a pharmaceutical agent administered. In certain embodiments, the pharmaceutical agent is Compound 696844, in certain embodiments, the pharmaceutical agent is a compound having the chemical structure of Compound 696844, or a pharmaceutically acceptable salt thereof.

As used herein, the term “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “loading dose” means a therapeutically effective amount of a pharmaceutical agent administered during an initial dosing phase during which target concentration of the pharmaceutical agent is achieved. “Initial loading dose” means the first loading dose administered. “Last loading dose” means the loading dose administered most recently prior to administering a first maintenance dose.

As used herein, “maintenance dose” means a therapeutically effective amount of a pharmaceutical agent administered during a dosing phase after target concentration of the pharmaceutical agent has been achieved and is maintained.

As used herein, the terms “FB-L_{Rx}” and “Compound 696844” are interchangeable.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methylcytosine” is a modified nucleobase. A universal base is a modified nucleobase that can pair with any one of the five unmodified nucleobases.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. “Linked nucleosides” are nucleosides that are connected in a contiguous sequence (i.e., no additional nucleosides are presented between those that are linked).

As used herein, “oligonucleotide” means a polymer of linked nucleosides connected via internucleoside linkages, wherein each nucleoside and internucleoside linkage may be modified or unmodified. Unless otherwise indicated, oligonucleotides consist of 8-50 linked nucleosides.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “pharmaceutical composition” means a mixture of substances suitable for administering to a subject. For example, a pharmaceutical composition may comprise an oligomeric compound and a sterile aqueous solution.

As used herein, “potassium salt” means a salt of a modified oligonucleotide or a compound, wherein the cation of the salt is potassium.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “RNA” means an RNA transcript and includes pre-mRNA and mature mRNA unless otherwise specified.

As used herein, “sodium salt” means a salt of a modified oligonucleotide or a compound, wherein the cation of the salt is sodium.

As used herein, “subject” means a human or non-human animal. In certain embodiments, the subject is a human subject. A “subject in need thereof,” is a subject who would benefit from administration of the compound disclosed herein.

As used herein, “sugar moiety” means an unmodified sugar moiety or a modified sugar moiety. “Unmodified sugar moiety” means a 2′-OH(H) β-D ribosyl moiety, as found in RNA (an “unmodified RNA sugar moiety”), or a 2′-H(H) β-D deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. “Modified sugar moiety” or “modified sugar” means a modified furanosyl sugar moiety or a sugar surrogate.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests. In certain embodiments, a hallmark is apparent on a brain MRI scan.

As used herein, “CFB RNA” is equivalent the RNA expression product of the human Factor B, “CFB RNA” may refer to pre-mRNA or mRNA.

As used herein, “therapeutically effective amount” means an amount of a pharmaceutical agent that provides a therapeutic benefit to a human subject. For example, a therapeutically effective amount improves a symptom or hallmark of a disease or disorder.

As used herein, “trough concentration” means the concentration of an analyte (e.g., CFB protein in a biological sample taken from a dosed human subject immediately prior to the human subject receiving a subsequent dose or the concentration of an analyte on the last study day.

As used herein, “week” means 7 days.

### Certain Embodiments

Embodiment 1. A method of ameliorating a disease or disorder associated with dysregulation of the alternative complement pathway in a human subject in need thereof, the method comprising administering to the human subject a therapeutically effective amount of a compound according to the following chemical structure:

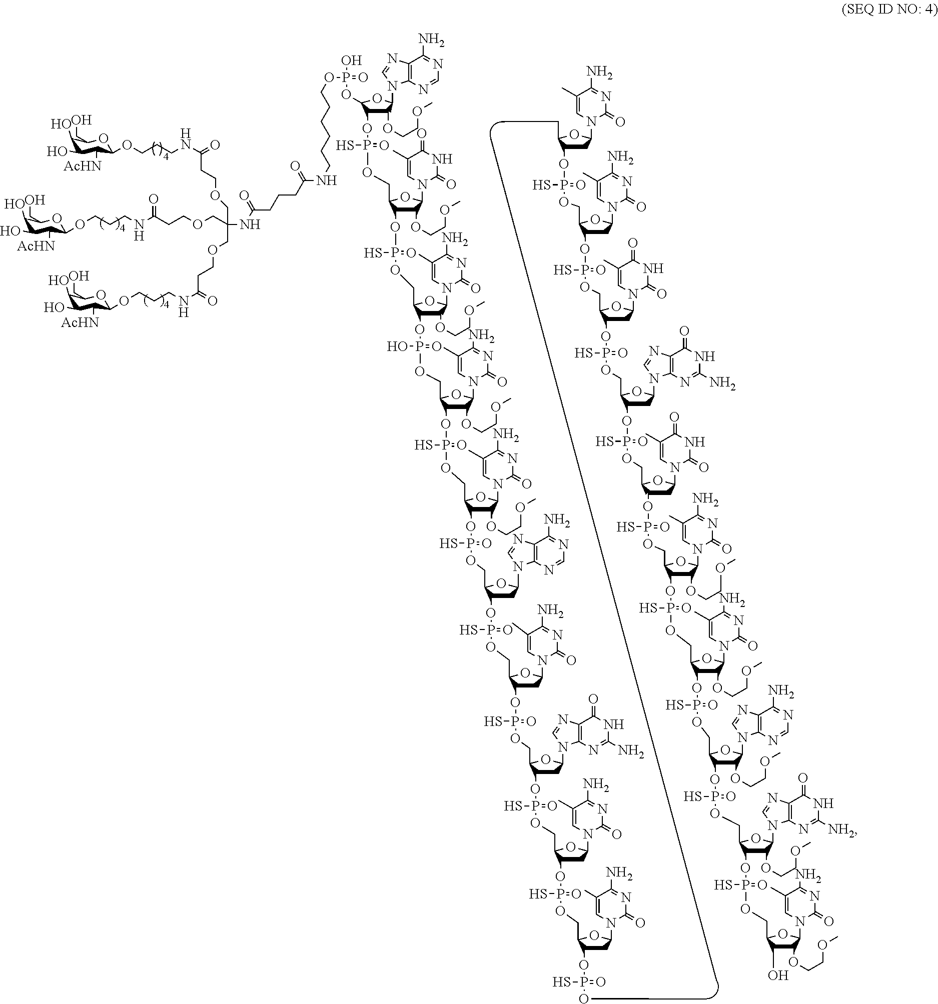

or a pharmaceutically acceptable salt thereof.

Embodiment 2. The method of embodiment 1, wherein the compound is the sodium salt or the potassium salt.

Embodiment 3. A method of ameliorating a disease or disorder associated with dysregulation of the alternative complement pathway in a human subject in need thereof, the method comprising administering to the human subject a therapeutically effective amount of a compound according to the following chemical structure:

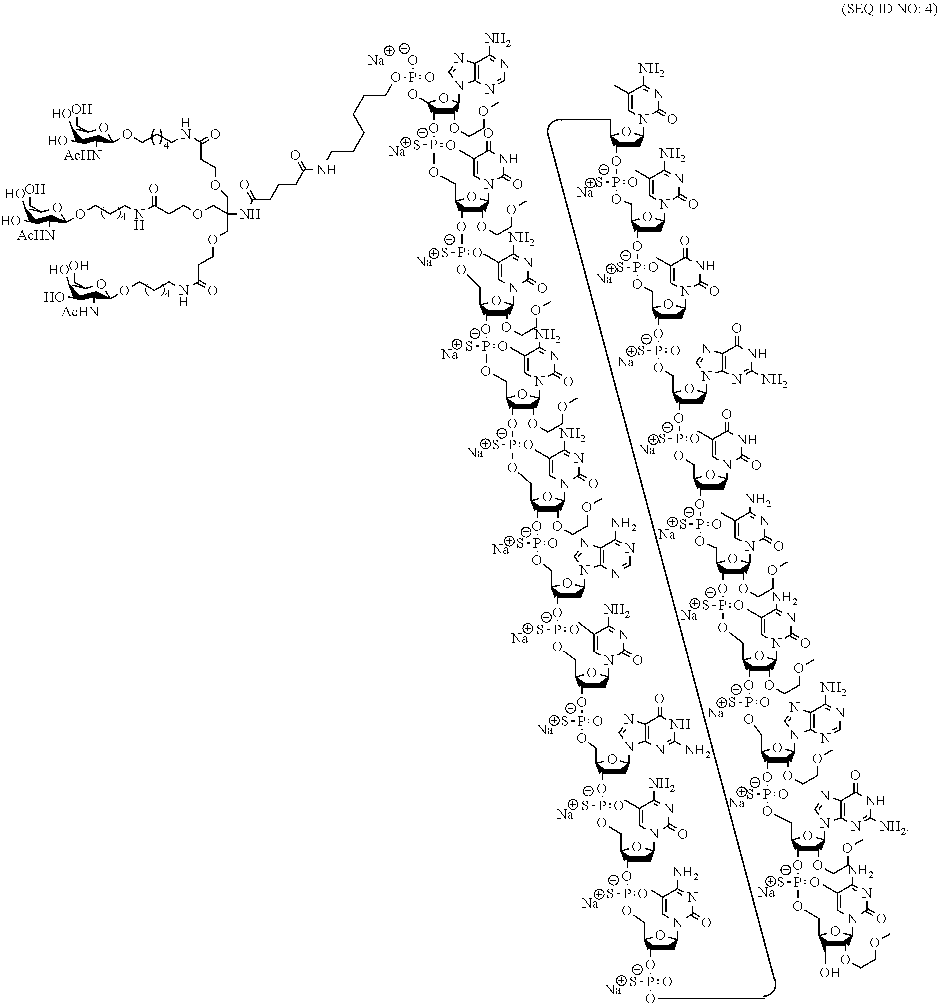

Embodiment 4. A method of ameliorating a disease or disorder associated with dysregulation of the alternative complement pathway in a human subject in need thereof, the method comprising administering to the human subject a therapeutically effective amount of a compound, wherein the compound has the following chemical notation (5′ to 3′): GalNAc₃-7_{a-o′} Aₑₛ Tₑₛ^{m}Cₑₛ^{m}Cₑₛ^{m}CₑₛA_{ds}^{m}C_{ds}G_{ds}^{m}C_{ds}^{m}C_{ds}^{m}C_{ds}^{m}C_{ds} T_{ds}G_{ds} T_{ds}^{m}Cₑₛ^{m}CₑₛAₑₛGₑₛ^{m}Cₑ (SEQ ID NO: 4); wherein,
A=an adenine nucleobase,
^{m}C=a 5-methylcytosine nucleobase,
G=a guanine nucleobase,
T=a thymine nucleobase,
e=a 2′-MOE sugar moiety,
d=a 2′-β-D-deoxyribosyl sugar moiety,
S=a phosphorothioate internucleoside linkage, and

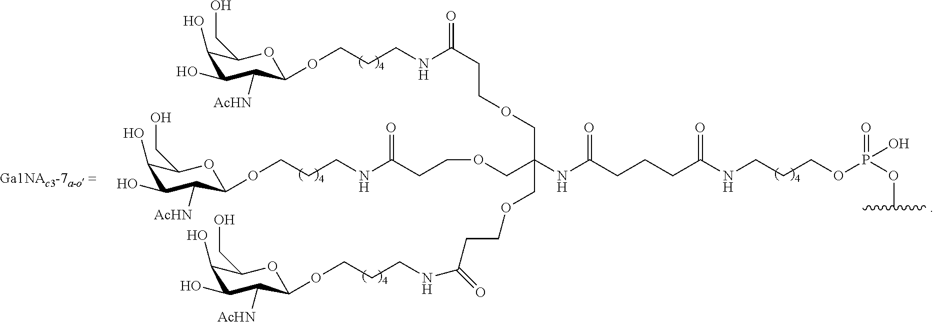

Embodiment 5. A method of reducing expression of Complement Factor B (CFB) RNA in a human subject having, or at risk of having, a disease or disorder associated with dysregulation of the alternative complement pathway, the method comprising administering to the human subject a therapeutically effective amount of a compound according to the following chemical structure:

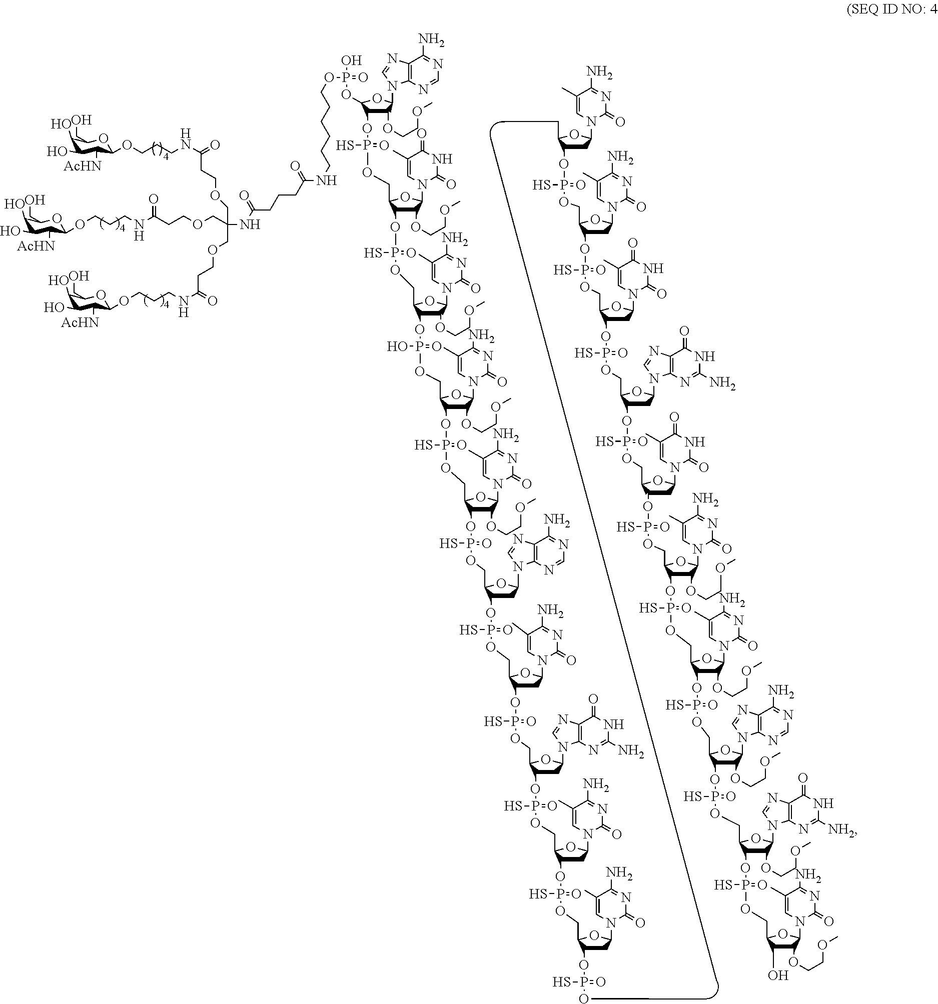

or a pharmaceutically acceptable salt thereof.

Embodiment 6. The method of embodiment 5, wherein the compound is the sodium salt or the potassium salt.

Embodiment 7. A method of reducing expression of Complement Factor B (CFB) RNA in a human subject having, or at risk of having, a disease or disorder associated with dysregulation of the alternative complement pathway, the method comprising administering to the human subject a therapeutically effective amount of a compound according to the following chemical structure:

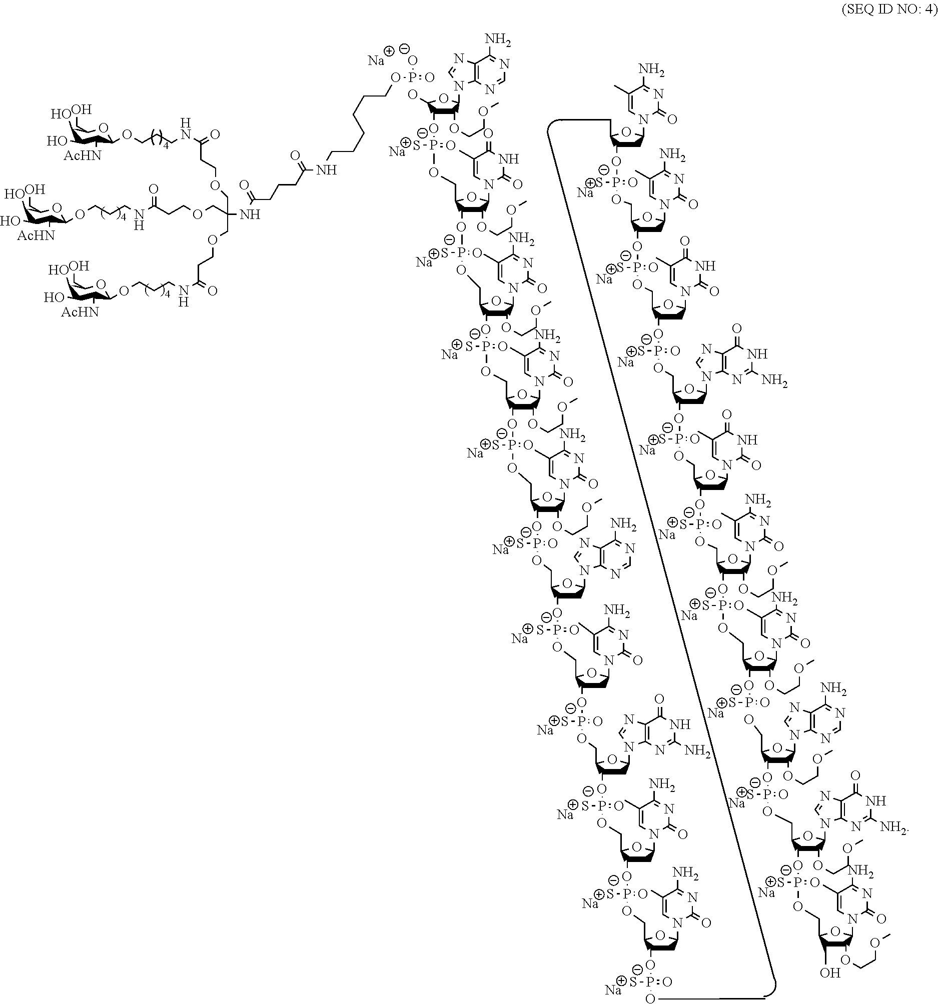

Embodiment 8. A method of reducing Complement Factor B (CFB) RNA in a human subject having, or at risk of having, a disease or disorder associated with dysregulation of the alternative complement pathway, the method comprising administering to the human subject a therapeutically effective amount of a compound, wherein the compound has the following chemical notation (5′ to 3′): GalNAc₃-7_{a-o′} Aₑₛ Tₑₛ^{m}Cₑₛ ^{m}Cₑₛ^{m}CₑₛA_{ds}^{m}C_{ds}G_{ds}^{m}C_{ds}^{m}C_{ds}^{m}C_{ds}^{m}C_{ds}T_{ds}G_{ds} T_{ds}^{m}Cₑₛ^{m}CₑₛAₑₛGₑₛ^{m}Cₑ (SEQ ID NO: 4); wherein,
A=an adenine nucleobase,
^{m}C=a 5-methylcytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and

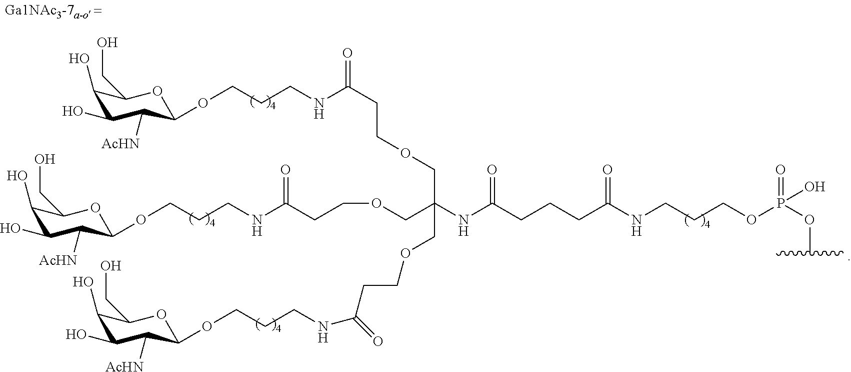

Embodiment 9. The method of any of embodiments 6-8, wherein CFB mRNA is reduced.

Embodiment 10. The method of any of embodiments 6-8, wherein CFB protein is reduced.

Embodiment 11. The method of any of embodiments 6-10, wherein administering the compound reduces CFB protein in one or both eyes.

Embodiment 12. The method of any of embodiments 6-11, wherein administering the compound reduces CFB protein in one or both kidneys.

Embodiment 13. The method of embodiment 12, wherein administering the compound reduces CFB protein in the glomerulus.

Embodiment 14. A method of reducing the level of a complement component in the eye or kidney of a human subject having, or at risk of having, a disease or disorder associated with dysregulation of the alternative complement pathway, the method comprising administering to the human subject a therapeutically effective amount of a compound according to the following chemical structure:

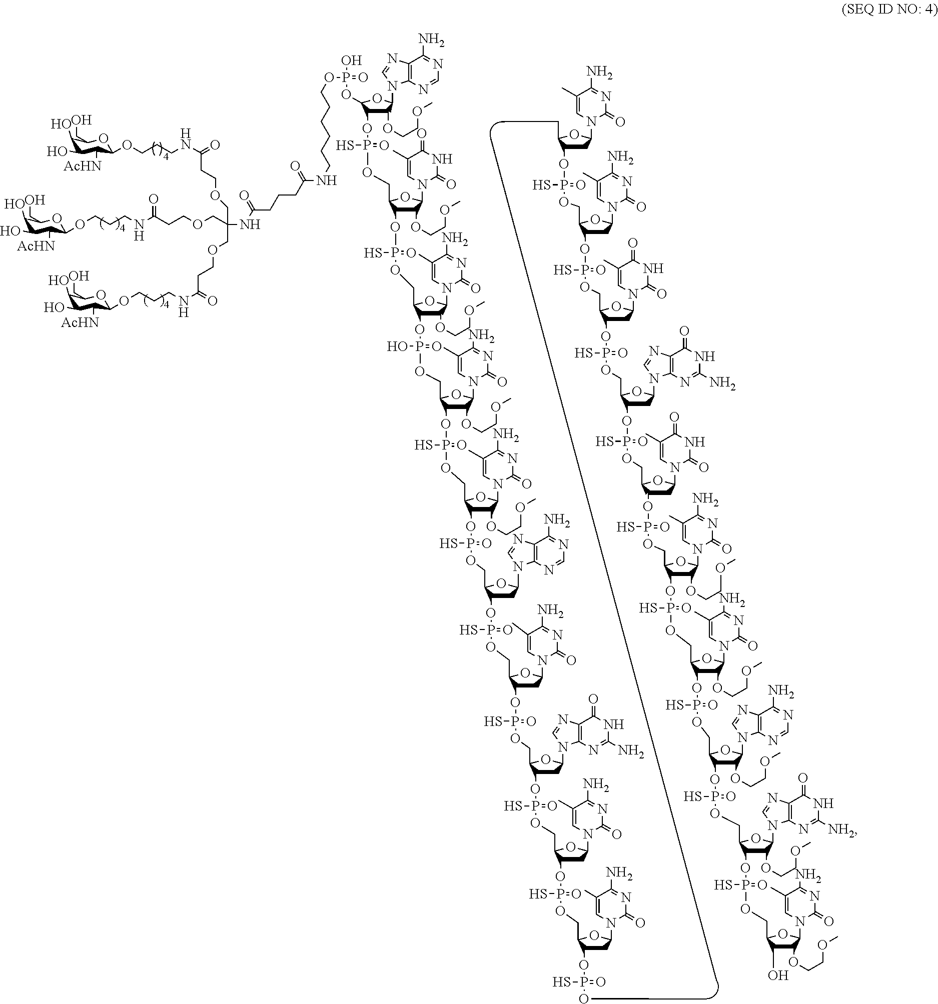

or a pharmaceutically acceptable salt thereof.

Embodiment 15. The method of embodiment 14, wherein the compound is the sodium salt or the potassium salt.

Embodiment 16. A method of reducing the level of a complement component in the eye or kidney of a human subject having, or at risk of having, a disease associated with dysregulation of the alternative complement pathway, the method comprising administering to the human subject a therapeutically effective amount of a compound according to the following chemical structure:

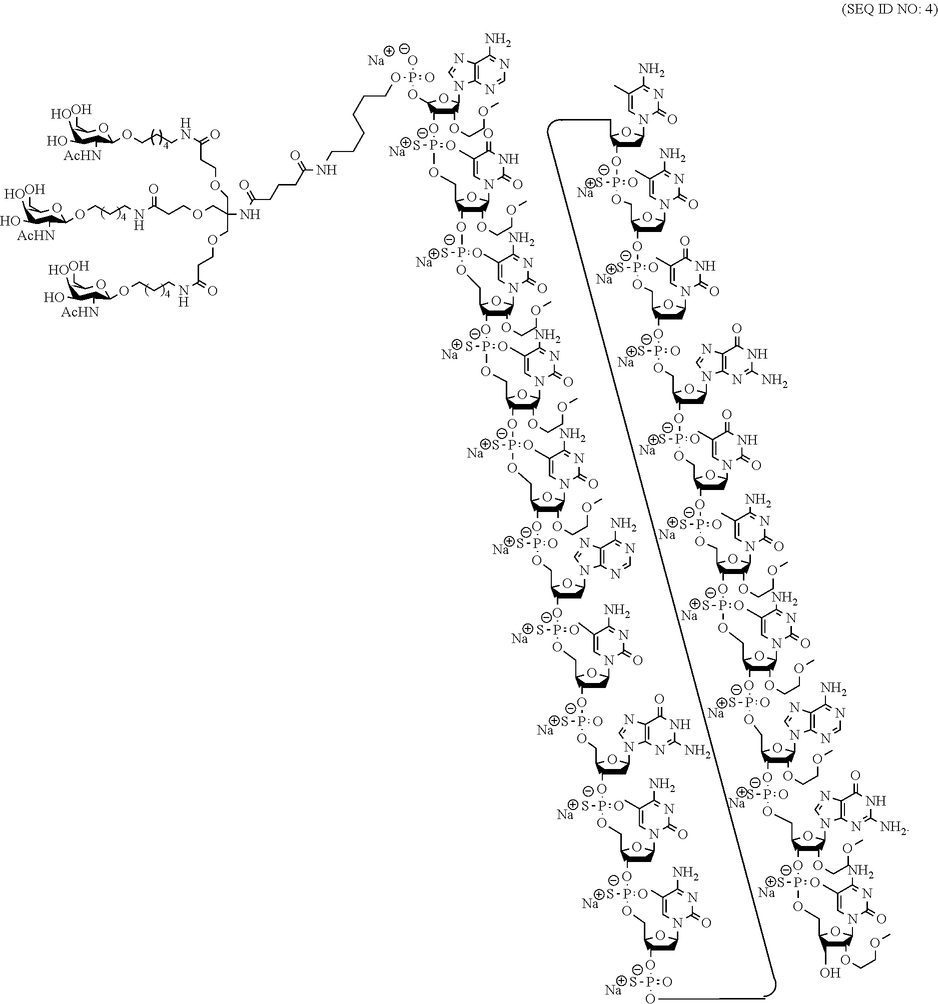

Embodiment 17. A method of reducing the level of a complement component in the eye or kidney of a human subject having, or at risk of having, a disease or disorder associated with dysregulation of the alternative complement pathway, the method comprising administering to the human subject a therapeutically effective amount of a compound, wherein the compound has the following chemical notation (5′ to 3′): GalNAc₃-7_{a-o′}
Aₑₛ Tₑₛ^{m}Cₑₛ^{m}Cₑₛ^{m}CₑₛA_{ds}^{m}C_{ds}G_{ds}^{m}C_{ds}^{m}C_{ds}^{m}C_{ds}^{m}C_{ds} T_{ds}G_{ds} T_{ds}^{m}Cₑₛ ^{m}CₑₛAₑₛGₑₛ^{m}Cₑ (SEQ ID NO: 4); wherein,
   A=an adenine nucleobase,
   ^{m}C=a 5-methylcytosine nucleobase,
   G=a guanine nucleobase,
   T=a thymine nucleobase,
   e=a 2′-MOE sugar moiety,
   d=a 2′-β-D-deoxyribosyl sugar moiety,
   S=a phosphorothioate internucleoside linkage, and

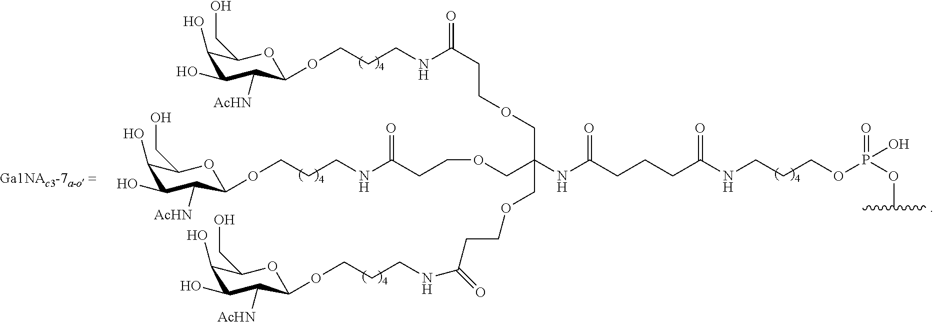

Embodiment 18. The method of any of embodiments 14-17, wherein the complement component is any of C3, C3a, C3b, C4b, C3dg, C5, C5a, C5b, C6, C7, C8, and C9.

Embodiment 19. A method of reducing Complement Factor B (CFB) protein in a human subject in need thereof, the method comprising administering to the human subject a therapeutically effective amount of a compound according to the following chemical structure:

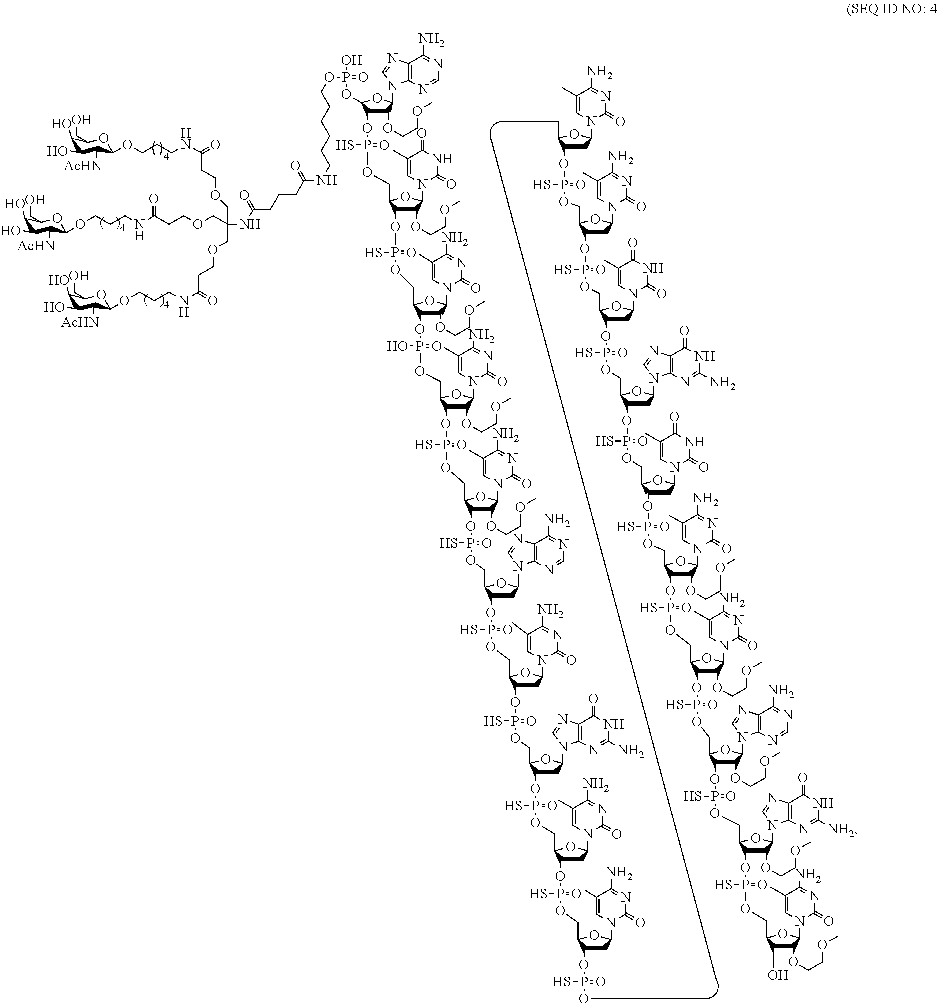

or a pharmaceutically acceptable salt thereof.

Embodiment 20. The method of embodiment 19, wherein the compound is the sodium salt or the potassium salt.

Embodiment 21. A method of reducing Complement Factor B (CFB) protein in a human subject in need thereof, the method comprising administering to the human subject a therapeutically effective amount of a compound according to the following chemical structure:

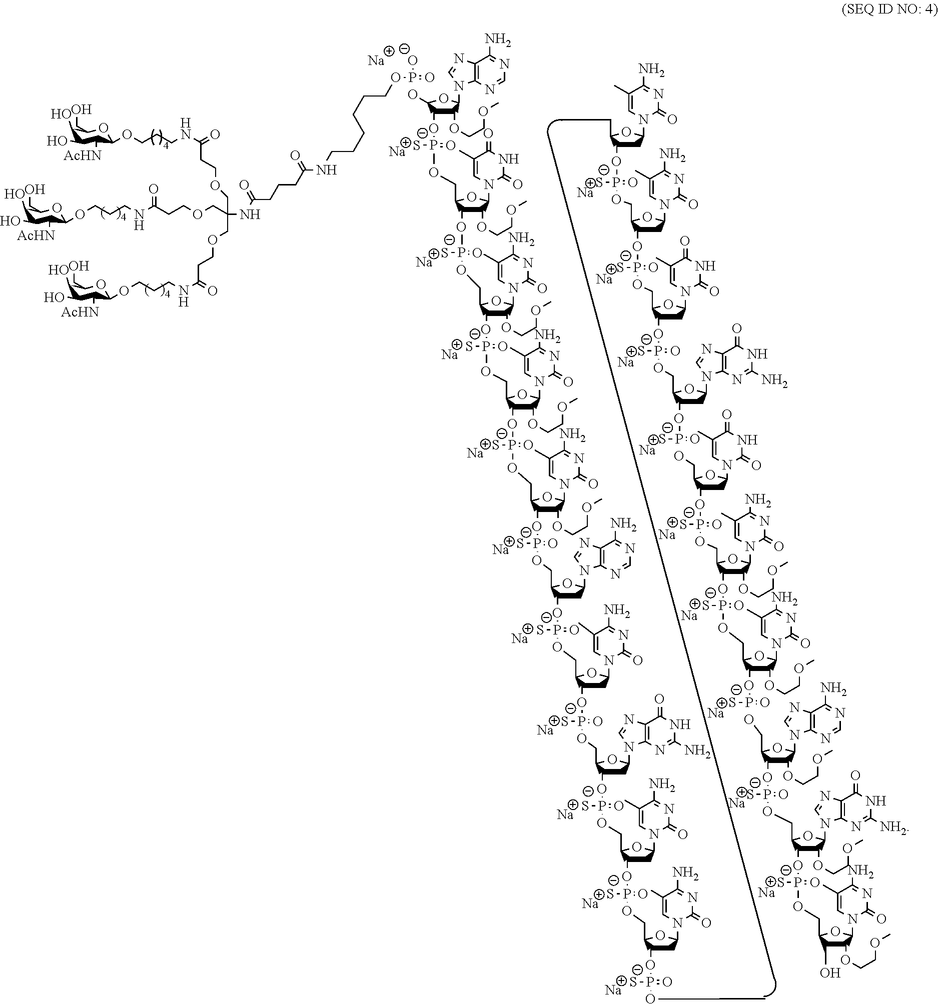

Embodiment 22. A method of reducing Complement Factor B (CFB) protein in a human subject in need thereof, the method comprising administering to the human subject a therapeutically effective amount of a compound, wherein the compound wherein the compound has the following chemical notation (5′ to 3′): GalNAc₃-7_{a-o′}
Aₑₛ Tₑₛ^{m}Cₑₛ^{m}Cₑₛ^{m}CₑₛA_{ds}^{m}C_{ds}G_{ds}^{m}C_{ds}^{m}C_{ds}^{m}C_{ds}^{m}C_{ds} T_{ds}G_{ds} T_{ds}^{m}Cₑₛ^{m}CₑₛAₑₛGₑₛ^{m}Cₑ (SEQ ID NO: 4);
wherein,
   A=an adenine nucleobase,
   ^{m}C=a 5-methylcytosine nucleobase,
   G=a guanine nucleobase,
   T=a thymine nucleobase,
   e=a 2′-MOE sugar moiety,
   d=a 2′-β-D-deoxyribosyl sugar moiety,
   s=a phosphorothioate internucleoside linkage, and

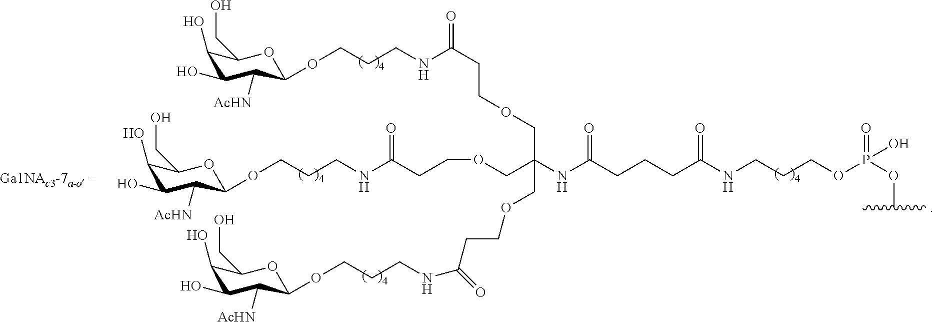

Embodiment 23. The method of any of embodiments 19-22, wherein the human subject has a disease or disorder associated with dysregulation of the alternative complement pathway.

Embodiment 24. The method of any of embodiments 1-23, wherein the activity of the complement pathway is elevated.

Embodiment 25. The method of any of embodiments 1-24, wherein the activity of the alternative complement activity is elevated.

Embodiment 26. The method of embodiment 24 or embodiment 25, wherein the elevated activity is associated with a disease or disorder in the human subject.

Embodiment 27. The method of any of embodiments 1-26, wherein reducing CFB protein ameliorates the disease or disorder.

Embodiment 28. The method of any of embodiments 1-18 or 23-27, wherein the disease or disorder is an ocular disease or disorder.

Embodiment 29. The method of any of embodiments 1-18 or 23-28, wherein the disease or disorder is macular degeneration, neuromyelitis optica; corneal disease, corneal inflammation; autoimmune uveitis; or diabetic retinopathy.

Embodiment 30. The method of embodiment 29, wherein the macular degeneration is age related macular degeneration (AMD)

Embodiment 31. The method of embodiment 30, wherein the AMD is wet AMD or intermediate AMD.

Embodiment 32. The method of embodiment 30, wherein the AMD is dry AMD.

Embodiment 33. The method of any of embodiments 1-18 or 23-32, wherein the disease or disorder is Geographic Atrophy associated with AMD.

Embodiment 34. The method of any of embodiments 1-18 or 23-27, wherein the disease or disorder is a kidney disease or disorder.

Embodiment 35. The method of embodiment 34, wherein the kidney disease or disorder is IgA Nephropathy.

Embodiment 36. The method of embodiment 34, wherein the disease is lupus nephritis, systemic lupus erythematosus (SLE), dense deposit disease (DDD), C3 glomerulonephritis (C3GN), CFHR5 nephropathy, or atypical hemolytic uremic syndrome (aHUS).

Embodiment 37. The method of embodiment 36, wherein the aHUS is characterized by thrombotic microangiopathy.

Embodiment 38. The method of any of embodiments 34-36, wherein the disease disorder is a kidney disease or disorder associated with C3 deposits.

Embodiment 39. The method of embodiment 38, wherein the kidney disease or disorder is associated with C3 deposits in the glomerulus.

Embodiment 40. The method of any of embodiments 34-39, wherein the disease or disorder is a kidney disease or disorder associated with lower than normal circulating C3 levels.

Embodiment 41. The method of embodiment 40, wherein the circulating C3 levels are serum or plasma C3 levels.

Embodiment 42. The method of any of embodiments 1-41, wherein administering the compound reduces accumulation of ocular C3 levels.

Embodiment 43. The method of embodiment 42, wherein the accumulation is in the vasculature.

Embodiment 44. The method of any of embodiments 1-41, wherein administering the compound reduces accumulation of C3 in the kidney.

Embodiment 45. The method of any of embodiments 1-41 or 44, wherein administering the compound reduces the level of C3 in the kidney, or reduces accumulation of kidney C3 deposits.

Embodiment 46. The method of any of embodiments 41 or 44-45, wherein administering the compound reduces the level of C3 or accumulation of C3 in the glomerulus.

Embodiment 47. The method of any of embodiments 1-18 or 23-27, wherein the disease or disorder is ANCA-associated vasculitis, antiphospholipid syndrome (also known as antiphospholipid antibody syndrome (APS)), asthma, rheumatoid arthritis, Myasthenia Gravis, multiple sclerosis, preeclampsia or a metabolic disorder (such as NASH).

Embodiment 48. The method of any of embodiments 1-47, wherein the subject is identified as having or at risk of having a disease or disorder associated with dysregulation of the alternative complement pathway.

Embodiment 49. The method of embodiment 48, wherein the identification comprises detecting complement levels or membrane-attack complex levels in the subject's blood.

Embodiment 50. The method of embodiment 49, wherein the identification comprises detecting complement levels or membrane-attack complex levels in the subject's serum or plasma.

Embodiment 51. The method of embodiment 49, wherein the identification comprises performing a genetic test for gene mutations of complement factors associated with the disease or disorder.

Embodiment 52. The method of any of embodiments 1-18 or 23-51, wherein at least one symptom or hallmark of the disease or disorder associated with dysregulation of the alternative complement pathway is ameliorated.

Embodiment 53. The method of embodiment 52, wherein the symptom or hallmark is proteinuria; progressive reduction in kidney function; C3 deposits in kidney; hematuria; hypertension; inflammation in kidney; IgA deposits in glomerular mesangium; hyper-active alternative complement pathway; elevated plasma CFB protein; elevated serum, plasma, or urine Bb; elevated serum, plasma, or urine Ba; elevated serum, plasma or urine sC5b-9; elevated or reduced serum, plasma, or urine C3; elevated serum, plasma, or urine C4; or elevated serum AH50 activity.

Embodiment 54. The method of embodiment 52, wherein the symptom or hallmark is progressive reduction in visual acuity; progressive reduction in low luminescence visual acuity; progressive reduction in best corrected visual acuity; progressive reduction in central vision; morphological changes in choriocapillaris; ocular geographic atrophy; ocular C3 deposits; hyper-active alternative complement pathway; elevated plasma CFB protein; elevated serum, plasma, or urine Bb; elevated serum, plasma, or urine Ba; elevated serum, plasma or urine sC5b-9; elevated or reduced serum, plasma, or urine C3; elevated serum, plasma, or urine C4; or elevated serum AH50 activity.

Embodiment 55. The method of any of embodiment 1-54, wherein administering the compound slows or prevents reduction of visual acuity; slows or prevents reduction of low luminescence visual acuity; slows or prevents reduction in best corrected visual acuity; slows or prevents reduction in central vision; slows or prevents morphological changes in choriocapillaris; slows or prevents ocular geographic atrophy; reduces ocular C3 deposits; reduces alternative complement pathway activity; reduces plasma CFB protein; reduces serum, plasma, or urine Bb; reduces serum, plasma, or urine Ba; reduces serum, plasma or urine sC5b-9; modulates, increases, or reduces serum, plasma, or urine C3; reduces serum, plasma, or urine C4; or reduces serum AH50 activity.

Embodiment 56. The method of any of embodiments 1-53, wherein administering the compound reduces proteinuria; slows or prevents reduction in kidney function; reduces C3 deposits in kidney; reduces hematuria; reduces hypertension; reduces inflammation in kidney; reduces IgA deposits in glomerular mesangium; reduces alternative complement pathway activity; reduces plasma CFB protein; reduces serum, plasma, or urine Bb; reduces serum, plasma, or urine Ba; reduces serum, plasma or urine sC5b-9; modulates, increases, or reduces serum, plasma, or urine C3; reduces serum, plasma, or urine C4; or reduces serum AH50 activity.

Embodiment 57. The method of any of embodiments 1-56, wherein the urine protein/creatine ratio is reduced following administration of the compound.

Embodiment 58. The method of any of embodiments 1-57, wherein the level of urine protein is reduced following administration of the compound.

Embodiment 59. The method of any of embodiments 28-33, wherein administering the compound slows or prevents the progression or development of Geographic Atrophy in the subject.

Embodiment 60. The method of any of embodiments 28-33, wherein administration of the compound slows the expansion of Geographic Atrophy Area in the subject.

Embodiment 61. The method of embodiment 60, wherein administration of the compound slows the expansion of Geographic Atrophy into the fovea region.

Embodiment 62. The method of any of embodiments 28-33, wherein administration of the compound stops expansion of Geographic Atrophy.

Embodiment 63. The method of any of embodiments 1-62, wherein the therapeutically effective amount is 10 mg.

Embodiment 64. The method of any of embodiments 1-62, wherein the therapeutically effective amount is 20 mg.

Embodiment 65. The method of any of embodiments 1-62, wherein the therapeutically effective amount is 40 mg.

Embodiment 66. The method of any of embodiments 1-62, wherein the therapeutically effective amount is 70 mg.

Embodiment 67. The method of any of embodiments 1-62, wherein the therapeutically effective amount is 100 mg.

Embodiment 68. The method of any of embodiments 1-62, wherein the therapeutically effective amount is about 10 mg.

Embodiment 69. The method of any of embodiments 1-62, wherein the therapeutically effective amount is about 20 mg.

Embodiment 70. The method of any of embodiments 1-62, wherein the therapeutically effective amount is about 40 mg.

Embodiment 71. The method of any of embodiments 1-62, wherein the therapeutically effective amount is about 70 mg.

Embodiment 72. The method of any of embodiments 1-62, wherein the therapeutically effective amount is about 100 mg.

Embodiment 73. The method of any of embodiments 1-62, wherein the therapeutically effective amount is any of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, and 350 mg.

Embodiment 74. The method of any of embodiments 1-62 wherein the therapeutically effective amount is any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, and about 350 mg.

Embodiment 75. The method of any of embodiments 1-62, wherein the therapeutically effective amount is any of 10 mg, 10.1 mg, 10.2 mg, 10.3 mg, 10.4 mg, 10.5 mg, 10.6 mg, 10.7 mg, 10.8 mg, 10.9 mg, 11 mg, 11.1 mg, 11.2 mg, 11.3 mg, 11.4 mg, 11.5 mg, 11.6 mg, 11.7 mg, 11.8 mg, 11.9 mg, 12 mg, 12.1 mg, 12.2 mg, 12.3 mg, 12.4 mg, 12.5 mg, 12.6 mg, 12.7 mg, 12.8 mg, 12.9 mg, 13 mg, 13.1 mg, 13.2 mg, 13.3 mg, 13.4 mg, 13.5 mg, 13.6 mg, 13.7 mg, 13.8 mg, 13.9 mg, 14 mg, 14.1 mg, 14.2 mg, 14.3 mg, 14.4 mg, 14.5 mg, 14.6 mg, 14.7 mg, 14.8 mg, 14.9 mg, 15 mg, 15.1 mg, 15.2 mg, 15.3 mg, 15.4 mg, 15.5 mg, 15.6 mg, 15.7 mg, 15.8 mg, 15.9 mg, 16 mg, 16.1 mg, 16.2 mg, 16.3 mg, 16.4 mg, 16.5 mg, 16.6 mg, 16.7 mg, 16.8 mg, 16.9 mg, 17 mg, 17.1 mg, 17.2 mg, 17.3 mg, 17.4 mg, 17.5 mg, 17.6 mg, 17.7 mg, 17.8 mg, 17.9 mg, 18 mg, 18.1 mg, 18.2 mg, 18.3 mg, 18.4 mg, 18.5 mg, 18.6 mg, 18.7 mg, 18.8 mg, 18.9 mg, 19 mg, 19.1 mg, 19.2 mg, 19.3 mg, 19.4 mg, 19.5 mg, 19.6 mg, 19.7 mg, 19.8 mg, 19.9 mg, 20 mg, 20.1 mg, 20.2 mg, 20.3 mg, 20.4 mg, 20.5 mg, 20.6 mg, 20.7 mg, 20.8 mg, 20.9 mg, 21 mg, 21.1 mg, 21.2 mg, 21.3 mg, 21.4 mg, 21.5 mg, 21.6 mg, 21.7 mg, 21.8 mg, 21.9 mg, 22 mg, 22.1 mg, 22.2 mg, 22.3 mg, 22.4 mg, 22.5 mg, 22.6 mg, 22.7 mg, 22.8 mg, 22.9 mg, 23 mg, 23.1 mg, 23.2 mg, 23.3 mg, 23.4 mg, 23.5 mg, 23.6 mg, 23.7 mg, 23.8 mg, 23.9 mg, 24 mg, 24.1 mg, 24.2 mg, 24.3 mg, 24.4 mg, 24.5 mg, 24.6 mg, 24.7 mg, 24.8 mg, 24.9 mg, 25 mg, 25.1 mg, 25.2 mg, 25.3 mg, 25.4 mg, 25.5 mg, 25.6 mg, 25.7 mg, 25.8 mg, 25.9 mg, 26 mg, 26.1 mg, 26.2 mg, 26.3 mg, 26.4 mg, 26.5 mg, 26.6 mg, 26.7 mg, 26.8 mg, 26.9 mg, 27 mg, 27.1 mg, 27.2 mg, 27.3 mg, 27.4 mg, 27.5 mg, 27.6 mg, 27.7 mg, 27.8 mg, 27.9 mg, 28 mg, 28.1 mg, 28.2 mg, 28.3 mg, 28.4 mg, 28.5 mg, 28.6 mg, 28.7 mg, 28.8 mg, 28.9 mg, 29 mg, 29.1 mg, 29.2 mg, 29.3 mg, 29.4 mg, 29.5 mg, 29.6 mg, 29.7 mg, 29.8 mg, 29.9 mg, 30 mg, 30.1 mg, 30.2 mg, 30.3 mg, 30.4 mg, 30.5 mg, 30.6 mg, 30.7 mg, 30.8 mg, 30.9 mg, 31 mg, 31.1 mg, 31.2 mg, 31.3 mg, 31.4 mg, 31.5 mg, 31.6 mg, 31.7 mg, 31.8 mg, 31.9 mg, 32 mg, 32.1 mg, 32.2 mg, 32.3 mg, 32.4 mg, 32.5 mg, 32.6 mg, 32.7 mg, 32.8 mg, 32.9 mg, 33 mg, 33.1 mg, 33.2 mg, 33.3 mg, 33.4 mg, 33.5 mg, 33.6 mg, 33.7 mg, 33.8 mg, 33.9 mg, 34 mg, 34.1 mg, 34.2 mg, 34.3 mg, 34.4 mg, 34.5 mg, 34.6 mg, 34.7 mg, 34.8 mg, 34.9 mg, 35 mg, 35.1 mg, 35.2 mg, 35.3 mg, 35.4 mg, 35.5 mg, 35.6 mg, 35.7 mg, 35.8 mg, 35.9 mg, 36 mg, 36.1 mg, 36.2 mg, 36.3 mg, 36.4 mg, 36.5 mg, 36.6 mg, 36.7 mg, 36.8 mg, 36.9 mg, 37 mg, 37.1 mg, 37.2 mg, 37.3 mg, 37.4 mg, 37.5 mg, 37.6 mg, 37.7 mg, 37.8 mg, 37.9 mg, 38 mg, 38.1 mg, 38.2 mg, 38.3 mg, 38.4 mg, 38.5 mg, 38.6 mg, 38.7 mg, 38.8 mg, 38.9 mg, 39 mg, 39.1 mg, 39.2 mg, 39.3 mg, 39.4 mg, 39.5 mg, 39.6 mg, 39.7 mg, 39.8 mg, 39.9 mg, 40 mg, 40.1 mg, 40.2 mg, 40.3 mg, 40.4 mg, 40.5 mg, 40.6 mg, 40.7 mg, 40.8 mg, 40.9 mg, 41 mg, 41.1 mg, 41.2 mg, 41.3 mg, 41.4 mg, 41.5 mg, 41.6 mg, 41.7 mg, 41.8 mg, 41.9 mg, 42 mg, 42.1 mg, 42.2 mg, 42.3 mg, 42.4 mg, 42.5 mg, 42.6 mg, 42.7 mg, 42.8 mg, 42.9 mg, 43 mg, 43.1 mg, 43.2 mg, 43.3 mg, 43.4 mg, 43.5 mg, 43.6 mg, 43.7 mg, 43.8 mg, 43.9 mg, 44 mg, 44.1 mg, 44.2 mg, 44.3 mg, 44.4 mg, 44.5 mg, 44.6 mg, 44.7 mg, 44.8 mg, 44.9 mg, 45 mg, 45.1 mg, 45.2 mg, 45.3 mg, 45.4 mg, 45.5 mg, 45.6 mg, 45.7 mg, 45.8 mg, 45.9 mg, 46 mg, 46.1 mg, 46.2 mg, 46.3 mg, 46.4 mg, 46.5 mg, 46.6 mg, 46.7 mg, 46.8 mg, 46.9 mg, 47 mg, 47.1 mg, 47.2 mg, 47.3 mg, 47.4 mg, 47.5 mg, 47.6 mg, 47.7 mg, 47.8 mg, 47.9 mg, 48 mg, 48.1 mg, 48.2 mg, 48.3 mg, 48.4 mg, 48.5 mg, 48.6 mg, 48.7 mg, 48.8 mg, 48.9 mg, 49 mg, 49.1 mg, 49.2 mg, 49.3 mg, 49.4 mg, 49.5 mg, 49.6 mg, 49.7 mg, 49.8 mg, 49.9 mg, 50 mg, 50.1 mg, 50.2 mg, 50.3 mg, 50.4 mg, 50.5 mg, 50.6 mg, 50.7 mg, 50.8 mg, 50.9 mg, 51 mg, 51.1 mg, 51.2 mg, 51.3 mg, 51.4 mg, 51.5 mg, 51.6 mg, 51.7 mg, 51.8 mg, 51.9 mg, 52 mg, 52.1 mg, 52.2 mg, 52.3 mg, 52.4 mg, 52.5 mg, 52.6 mg, 52.7 mg, 52.8 mg, 52.9 mg, 53 mg, 53.1 mg, 53.2 mg, 53.3 mg, 53.4 mg, 53.5 mg, 53.6 mg, 53.7 mg, 53.8 mg, 53.9 mg, 54 mg, 54.1 mg, 54.2 mg, 54.3 mg, 54.4 mg, 54.5 mg, 54.6 mg, 54.7 mg, 54.8 mg, 54.9 mg, 55 mg, 55.1 mg, 55.2 mg, 55.3 mg, 55.4 mg, 55.5 mg, 55.6 mg, 55.7 mg, 55.8 mg, 55.9 mg, 56 mg, 56.1 mg, 56.2 mg, 56.3 mg, 56.4 mg, 56.5 mg, 56.6 mg, 56.7 mg, 56.8 mg, 56.9 mg, 57 mg, 57.1 mg, 57.2 mg, 57.3 mg, 57.4 mg, 57.5 mg, 57.6 mg, 57.7 mg, 57.8 mg, 57.9 mg, 58 mg, 58.1 mg, 58.2 mg, 58.3 mg, 58.4 mg, 58.5 mg, 58.6 mg, 58.7 mg, 58.8 mg, 58.9 mg, 59 mg, 59.1 mg, 59.2 mg, 59.3 mg, 59.4 mg, 59.5 mg, 59.6 mg, 59.7 mg, 59.8 mg, 59.9 mg, 60 mg, 60.1 mg, 60.2 mg, 60.3 mg, 60.4 mg, 60.5 mg, 60.6 mg, 60.7 mg, 60.8 mg, 60.9 mg, 61 mg, 61.1 mg, 61.2 mg, 61.3 mg, 61.4 mg, 61.5 mg, 61.6 mg, 61.7 mg, 61.8 mg, 61.9 mg, 62 mg, 62.1 mg, 62.2 mg, 62.3 mg, 62.4 mg, 62.5 mg, 62.6 mg, 62.7 mg, 62.8 mg, 62.9 mg, 63 mg, 63.1 mg, 63.2 mg, 63.3 mg, 63.4 mg, 63.5 mg, 63.6 mg, 63.7 mg, 63.8 mg, 63.9 mg, 64 mg, 64.1 mg, 64.2 mg, 64.3 mg, 64.4 mg, 64.5 mg, 64.6 mg, 64.7 mg, 64.8 mg, 64.9 mg, 65 mg, 65.1 mg, 65.2 mg, 65.3 mg, 65.4 mg, 65.5 mg, 65.6 mg, 65.7 mg, 65.8 mg, 65.9 mg, 66 mg, 66.1 mg, 66.2 mg, 66.3 mg, 66.4 mg, 66.5 mg, 66.6 mg, 66.7 mg, 66.8 mg, 66.9 mg, 67 mg, 67.1 mg, 67.2 mg, 67.3 mg, 67.4 mg, 67.5 mg, 67.6 mg, 67.7 mg, 67.8 mg, 67.9 mg, 68 mg, 68.1 mg, 68.2 mg, 68.3 mg, 68.4 mg, 68.5 mg, 68.6 mg, 68.7 mg, 68.8 mg, 68.9 mg, 69 mg, 69.1 mg, 69.2 mg, 69.3 mg, 69.4 mg, 69.5 mg, 69.6 mg, 69.7 mg, 69.8 mg, and 69.9 mg.

Embodiment 76. The method of any of embodiments 1-62, wherein the therapeutically effective amount is any of about 10 mg, about 10.1 mg, about 10.2 mg, about 10.3 mg, about 10.4 mg, about 10.5 mg, about 10.6 mg, about 10.7 mg, about 10.8 mg, about 10.9 mg, about 11 mg, about 11.1 mg, about 11.2 mg, about 11.3 mg, about 11.4 mg, about 11.5 mg, about 11.6 mg, about 11.7 mg, about 11.8 mg, about 11.9 mg, about 12 mg, about 12.1 mg, about 12.2 mg, about 12.3 mg, about 12.4 mg, about 12.5 mg, about 12.6 mg, about 12.7 mg, about 12.8 mg, about 12.9 mg, about 13 mg, about 13.1 mg, about 13.2 mg, about 13.3 mg, about 13.4 mg, about 13.5 mg, about 13.6 mg, about 13.7 mg, about 13.8 mg, about 13.9 mg, about 14 mg, about 14.1 mg, about 14.2 mg, about 14.3 mg, about 14.4 mg, about 14.5 mg, about 14.6 mg, about 14.7 mg, about 14.8 mg, about 14.9 mg, about 15 mg, about 15.1 mg, about 15.2 mg, about 15.3 mg, about 15.4 mg, about 15.5 mg, about 15.6 mg, about 15.7 mg, about 15.8 mg, about 15.9 mg, about 16 mg, about 16.1 mg, about 16.2 mg, about 16.3 mg, about 16.4 mg, about 16.5 mg, about 16.6 mg, about 16.7 mg, about 16.8 mg, about 16.9 mg, about 17 mg, about 17.1 mg, about 17.2 mg, about 17.3 mg, about 17.4 mg, about 17.5 mg, about 17.6 mg, about 17.7 mg, about 17.8 mg, about 17.9 mg, about 18 mg, about 18.1 mg, about 18.2 mg, about 18.3 mg, about 18.4 mg, about 18.5 mg, about 18.6 mg, about 18.7 mg, about 18.8 mg, about 18.9 mg, about 19 mg, about 19.1 mg, about 19.2 mg, about 19.3 mg, about 19.4 mg, about 19.5 mg, about 19.6 mg, about 19.7 mg, about 19.8 mg, about 19.9 mg, about 20 mg, about 20.1 mg, about 20.2 mg, about 20.3 mg, about 20.4 mg, about 20.5 mg, about 20.6 mg, about 20.7 mg, about 20.8 mg, about 20.9 mg, about 21 mg, about 21.1 mg, about 21.2 mg, about 21.3 mg, about 21.4 mg, about 21.5 mg, about 21.6 mg, about 21.7 mg, about 21.8 mg, about 21.9 mg, about 22 mg, about 22.1 mg, about 22.2 mg, about 22.3 mg, about 22.4 mg, about 22.5 mg, about 22.6 mg, about 22.7 mg, about 22.8 mg, about 22.9 mg, about 23 mg, about 23.1 mg, about 23.2 mg, about 23.3 mg, about 23.4 mg, about 23.5 mg, about 23.6 mg, about 23.7 mg, about 23.8 mg, about 23.9 mg, about 24 mg, about 24.1 mg, about 24.2 mg, about 24.3 mg, about 24.4 mg, about 24.5 mg, about 24.6 mg, about 24.7 mg, about 24.8 mg, about 24.9 mg, about 25 mg, about 25.1 mg, about 25.2 mg, about 25.3 mg, about 25.4 mg, about 25.5 mg, about 25.6 mg, about 25.7 mg, about 25.8 mg, about 25.9 mg, about 26 mg, about 26.1 mg, about 26.2 mg, about 26.3 mg, about 26.4 mg, about 26.5 mg, about 26.6 mg, about 26.7 mg, about 26.8 mg, about 26.9 mg, about 27 mg, about 27.1 mg, about 27.2 mg, about 27.3 mg, about 27.4 mg, about 27.5 mg, about 27.6 mg, about 27.7 mg, about 27.8 mg, about 27.9 mg, about 28 mg, about 28.1 mg, about 28.2 mg, about 28.3 mg, about 28.4 mg, about 28.5 mg, about 28.6 mg, about 28.7 mg, about 28.8 mg, about 28.9 mg, about 29 mg, about 29.1 mg, about 29.2 mg, about 29.3 mg, about 29.4 mg, about 29.5 mg, about 29.6 mg, about 29.7 mg, about 29.8 mg, about 29.9 mg, about 30 mg, about 30.1 mg, about 30.2 mg, about 30.3 mg, about 30.4 mg, about 30.5 mg, about 30.6 mg, about 30.7 mg, about 30.8 mg, about 30.9 mg, about 31 mg, about 31.1 mg, about 31.2 mg, about 31.3 mg, about 31.4 mg, about 31.5 mg, about 31.6 mg, about 31.7 mg, about 31.8 mg, about 31.9 mg, about 32 mg, about 32.1 mg, about 32.2 mg, about 32.3 mg, about 32.4 mg, about 32.5 mg, about 32.6 mg, about 32.7 mg, about 32.8 mg, about 32.9 mg, about 33 mg, about 33.1 mg, about 33.2 mg, about 33.3 mg, about 33.4 mg, about 33.5 mg, about 33.6 mg, about 33.7 mg, about 33.8 mg, about 33.9 mg, about 34 mg, about 34.1 mg, about 34.2 mg, about 34.3 mg, about 34.4 mg, about 34.5 mg, about 34.6 mg, about 34.7 mg, about 34.8 mg, about 34.9 mg, about 35 mg, about 35.1 mg, about 35.2 mg, about 35.3 mg, about 35.4 mg, about 35.5 mg, about 35.6 mg, about 35.7 mg, about 35.8 mg, about 35.9 mg, about 36 mg, about 36.1 mg, about 36.2 mg, about 36.3 mg, about 36.4 mg, about 36.5 mg, about 36.6 mg, about 36.7 mg, about 36.8 mg, about 36.9 mg, about 37 mg, about 37.1 mg, about 37.2 mg, about 37.3 mg, about 37.4 mg, about 37.5 mg, about 37.6 mg, about 37.7 mg, about 37.8 mg, about 37.9 mg, about 38 mg, about 38.1 mg, about 38.2 mg, about 38.3 mg, about 38.4 mg, about 38.5 mg, about 38.6 mg, about 38.7 mg, about 38.8 mg, about 38.9 mg, about 39 mg, about 39.1 mg, about 39.2 mg, about 39.3 mg, about 39.4 mg, about 39.5 mg, about 39.6 mg, about 39.7 mg, about 39.8 mg, about 39.9 mg, about 40 mg, about 40.1 mg, about 40.2 mg, about 40.3 mg, about 40.4 mg, about 40.5 mg, about 40.6 mg, about 40.7 mg, about 40.8 mg, about 40.9 mg, about 41 mg, about 41.1 mg, about 41.2 mg, about 41.3 mg, about 41.4 mg, about 41.5 mg, about 41.6 mg, about 41.7 mg, about 41.8 mg, about 41.9 mg, about 42 mg, about 42.1 mg, about 42.2 mg, about 42.3 mg, about 42.4 mg, about 42.5 mg, about 42.6 mg, about 42.7 mg, about 42.8 mg, about 42.9 mg, about 43 mg, about 43.1 mg, about 43.2 mg, about 43.3 mg, about 43.4 mg, about 43.5 mg, about 43.6 mg, about 43.7 mg, about 43.8 mg, about 43.9 mg, about 44 mg, about 44.1 mg, about 44.2 mg, about 44.3 mg, about 44.4 mg, about 44.5 mg, about 44.6 mg, about 44.7 mg, about 44.8 mg, about 44.9 mg, about 45 mg, about 45.1 mg, about 45.2 mg, about 45.3 mg, about 45.4 mg, about 45.5 mg, about 45.6 mg, about 45.7 mg, about 45.8 mg, about 45.9 mg, about 46 mg, about 46.1 mg, about 46.2 mg, about 46.3 mg, about 46.4 mg, about 46.5 mg, about 46.6 mg, about 46.7 mg, about 46.8 mg, about 46.9 mg, about 47 mg, about 47.1 mg, about 47.2 mg, about 47.3 mg, about 47.4 mg, about 47.5 mg, about 47.6 mg, about 47.7 mg, about 47.8 mg, about 47.9 mg, about 48 mg, about 48.1 mg, about 48.2 mg, about 48.3 mg, about 48.4 mg, about 48.5 mg, about 48.6 mg, about 48.7 mg, about 48.8 mg, about 48.9 mg, about 49 mg, about 49.1 mg, about 49.2 mg, about 49.3 mg, about 49.4 mg, about 49.5 mg, about 49.6 mg, about 49.7 mg, about 49.8 mg, about 49.9 mg, about 50 mg, about 50.1 mg, about 50.2 mg, about 50.3 mg, about 50.4 mg, about 50.5 mg, about 50.6 mg, about 50.7 mg, about 50.8 mg, about 50.9 mg, about 51 mg, about 51.1 mg, about 51.2 mg, about 51.3 mg, about 51.4 mg, about 51.5 mg, about 51.6 mg, about 51.7 mg, about 51.8 mg, about 51.9 mg, about 52 mg, about 52.1 mg, about 52.2 mg, about 52.3 mg, about 52.4 mg, about 52.5 mg, about 52.6 mg, about 52.7 mg, about 52.8 mg, about 52.9 mg, about 53 mg, about 53.1 mg, about 53.2 mg, about 53.3 mg, about 53.4 mg, about 53.5 mg, about 53.6 mg, about 53.7 mg, about 53.8 mg, about 53.9 mg, about 54 mg, about 54.1 mg, about 54.2 mg, about 54.3 mg, about 54.4 mg, about 54.5 mg, about 54.6 mg, about 54.7 mg, about 54.8 mg, about 54.9 mg, about 55 mg, about 55.1 mg, about 55.2 mg, about 55.3 mg, about 55.4 mg, about 55.5 mg, about 55.6 mg, about 55.7 mg, about 55.8 mg, about 55.9 mg, about 56 mg, about 56.1 mg, about 56.2 mg, about 56.3 mg, about 56.4 mg, about 56.5 mg, about 56.6 mg, about 56.7 mg, about 56.8 mg, about 56.9 mg, about 57 mg, about 57.1 mg, about 57.2 mg, about 57.3 mg, about 57.4 mg, about 57.5 mg, about 57.6 mg, about 57.7 mg, about 57.8 mg, about 57.9 mg, about 58 mg, about 58.1 mg, about 58.2 mg, about 58.3 mg, about 58.4 mg, about 58.5 mg, about 58.6 mg, about 58.7 mg, about 58.8 mg, about 58.9 mg, about 59 mg, about 59.1 mg, about 59.2 mg, about 59.3 mg, about 59.4 mg, about 59.5 mg, about 59.6 mg, about 59.7 mg, about 59.8 mg, about 59.9 mg, about 60 mg, about 60.1 mg, about 60.2 mg, about 60.3 mg, about 60.4 mg, about 60.5 mg, about 60.6 mg, about 60.7 mg, about 60.8 mg, about 60.9 mg, about 61 mg, about 61.1 mg, about 61.2 mg, about 61.3 mg, about 61.4 mg, about 61.5 mg, about 61.6 mg, about 61.7 mg, about 61.8 mg, about 61.9 mg, about 62 mg, about 62.1 mg, about 62.2 mg, about 62.3 mg, about 62.4 mg, about 62.5 mg, about 62.6 mg, about 62.7 mg, about 62.8 mg, about 62.9 mg, about 63 mg, about 63.1 mg, about 63.2 mg, about 63.3 mg, about 63.4 mg, about 63.5 mg, about 63.6 mg, about 63.7 mg, about 63.8 mg, about 63.9 mg, about 64 mg, about 64.1 mg, about 64.2 mg, about 64.3 mg, about 64.4 mg, about 64.5 mg, about 64.6 mg, about 64.7 mg, about 64.8 mg, about 64.9 mg, about 65 mg, about 65.1 mg, about 65.2 mg, about 65.3 mg, about 65.4 mg, about 65.5 mg, about 65.6 mg, about 65.7 mg, about 65.8 mg, about 65.9 mg, about 66 mg, about 66.1 mg, about 66.2 mg, about 66.3 mg, about 66.4 mg, about 66.5 mg, about 66.6 mg, about 66.7 mg, about 66.8 mg, about 66.9 mg, about 67 mg, about 67.1 mg, about 67.2 mg, about 67.3 mg, about 67.4 mg, about 67.5 mg, about 67.6 mg, about 67.7 mg, about 67.8 mg, about 67.9 mg, about 68 mg, about 68.1 mg, about 68.2 mg, about 68.3 mg, about 68.4 mg, about 68.5 mg, about 68.6 mg, about 68.7 mg, about 68.8 mg, about 68.9 mg, about 69 mg, about 69.1 mg, about 69.2 mg, about 69.3 mg, about 69.4 mg, about 69.5 mg, about 69.6 mg, about 69.7 mg, about 69.8 mg, and about 69.9 mg.

Embodiment 77. The method of any of embodiments 1-62, wherein the therapeutically effective amount is any of 20 mg, 20.1 mg, 20.2 mg, 20.3 mg, 20.4 mg, 20.5 mg, 20.6 mg, 20.7 mg, 20.8 mg, 20.9 mg, 21 mg, 21.1 mg, 21.2 mg, 21.3 mg, 21.4 mg, 21.5 mg, 21.6 mg, 21.7 mg, 21.8 mg, 21.9 mg, 22 mg, 22.1 mg, 22.2 mg, 22.3 mg, 22.4 mg, 22.5 mg, 22.6 mg, 22.7 mg, 22.8 mg, 22.9 mg, 23 mg, 23.1 mg, 23.2 mg, 23.3 mg, 23.4 mg, 23.5 mg, 23.6 mg, 23.7 mg, 23.8 mg, 23.9 mg, 24 mg, 24.1 mg, 24.2 mg, 24.3 mg, 24.4 mg, 24.5 mg, 24.6 mg, 24.7 mg, 24.8 mg, 24.9 mg, 25 mg, 25.1 mg, 25.2 mg, 25.3 mg, 25.4 mg, 25.5 mg, 25.6 mg, 25.7 mg, 25.8 mg, 25.9 mg, 26 mg, 26.1 mg, 26.2 mg, 26.3 mg, 26.4 mg, 26.5 mg, 26.6 mg, 26.7 mg, 26.8 mg, 26.9 mg, 27 mg, 27.1 mg, 27.2 mg, 27.3 mg, 27.4 mg, 27.5 mg, 27.6 mg, 27.7 mg, 27.8 mg, 27.9 mg, 28 mg, 28.1 mg, 28.2 mg, 28.3 mg, 28.4 mg, 28.5 mg, 28.6 mg, 28.7 mg, 28.8 mg, 28.9 mg, 29 mg, 29.1 mg, 29.2 mg, 29.3 mg, 29.4 mg, 29.5 mg, 29.6 mg, 29.7 mg, 29.8 mg, 29.9 mg, 30 mg, 30.1 mg, 30.2 mg, 30.3 mg, 30.4 mg, 30.5 mg, 30.6 mg, 30.7 mg, 30.8 mg, 30.9 mg, 31 mg, 31.1 mg, 31.2 mg, 31.3 mg, 31.4 mg, 31.5 mg, 31.6 mg, 31.7 mg, 31.8 mg, 31.9 mg, 32 mg, 32.1 mg, 32.2 mg, 32.3 mg, 32.4 mg, 32.5 mg, 32.6 mg, 32.7 mg, 32.8 mg, 32.9 mg, 33 mg, 33.1 mg, 33.2 mg, 33.3 mg, 33.4 mg, 33.5 mg, 33.6 mg, 33.7 mg, 33.8 mg, 33.9 mg, 34 mg, 34.1 mg, 34.2 mg, 34.3 mg, 34.4 mg, 34.5 mg, 34.6 mg, 34.7 mg, 34.8 mg, 34.9 mg, 35 mg, 35.1 mg, 35.2 mg, 35.3 mg, 35.4 mg, 35.5 mg, 35.6 mg, 35.7 mg, 35.8 mg, 35.9 mg, 36 mg, 36.1 mg, 36.2 mg, 36.3 mg, 36.4 mg, 36.5 mg, 36.6 mg, 36.7 mg, 36.8 mg, 36.9 mg, 37 mg, 37.1 mg, 37.2 mg, 37.3 mg, 37.4 mg, 37.5 mg, 37.6 mg, 37.7 mg, 37.8 mg, 37.9 mg, 38 mg, 38.1 mg, 38.2 mg, 38.3 mg, 38.4 mg, 38.5 mg, 38.6 mg, 38.7 mg, 38.8 mg, 38.9 mg, 39 mg, 39.1 mg, 39.2 mg, 39.3 mg, 39.4 mg, 39.5 mg, 39.6 mg, 39.7 mg, 39.8 mg, 39.9 mg, and 40 mg.

Embodiment 78. The method of any of embodiments 1-62, wherein the therapeutically effective amount is any of about 20 mg, about 20.1 mg, about 20.2 mg, about 20.3 mg, about 20.4 mg, about 20.5 mg, about 20.6 mg, about 20.7 mg, about 20.8 mg, about 20.9 mg, about 21 mg, about 21.1 mg, about 21.2 mg, about 21.3 mg, about 21.4 mg, about 21.5 mg, about 21.6 mg, about 21.7 mg, about 21.8 mg, about 21.9 mg, about 22 mg, about 22.1 mg, about 22.2 mg, about 22.3 mg, about 22.4 mg, about 22.5 mg, about 22.6 mg, about 22.7 mg, about 22.8 mg, about 22.9 mg, about 23 mg, about 23.1 mg, about 23.2 mg, about 23.3 mg, about 23.4 mg, about 23.5 mg, about 23.6 mg, about 23.7 mg, about 23.8 mg, about 23.9 mg, about 24 mg, about 24.1 mg, about 24.2 mg, about 24.3 mg, about 24.4 mg, about 24.5 mg, about 24.6 mg, about 24.7 mg, about 24.8 mg, about 24.9 mg, about 25 mg, about 25.1 mg, about 25.2 mg, about 25.3 mg, about 25.4 mg, about 25.5 mg, about 25.6 mg, about 25.7 mg, about 25.8 mg, about 25.9 mg, about 26 mg, about 26.1 mg, about 26.2 mg, about 26.3 mg, about 26.4 mg, about 26.5 mg, about 26.6 mg, about 26.7 mg, about 26.8 mg, about 26.9 mg, about 27 mg, about 27.1 mg, about 27.2 mg, about 27.3 mg, about 27.4 mg, about 27.5 mg, about 27.6 mg, about 27.7 mg, about 27.8 mg, about 27.9 mg, about 28 mg, about 28.1 mg, about 28.2 mg, about 28.3 mg, about 28.4 mg, about 28.5 mg, about 28.6 mg, about 28.7 mg, about 28.8 mg, about 28.9 mg, about 29 mg, about 29.1 mg, about 29.2 mg, about 29.3 mg, about 29.4 mg, about 29.5 mg, about 29.6 mg, about 29.7 mg, about 29.8 mg, about 29.9 mg, about 30 mg, about 30.1 mg, about 30.2 mg, about 30.3 mg, about 30.4 mg, about 30.5 mg, about 30.6 mg, about 30.7 mg, about 30.8 mg, about 30.9 mg, about 31 mg, about 31.1 mg, about 31.2 mg, about 31.3 mg, about 31.4 mg, about 31.5 mg, about 31.6 mg, about 31.7 mg, about 31.8 mg, about 31.9 mg, about 32 mg, about 32.1 mg, about 32.2 mg, about 32.3 mg, about 32.4 mg, about 32.5 mg, about 32.6 mg, about 32.7 mg, about 32.8 mg, about 32.9 mg, about 33 mg, about 33.1 mg, about 33.2 mg, about 33.3 mg, about 33.4 mg, about 33.5 mg, about 33.6 mg, about 33.7 mg, about 33.8 mg, about 33.9 mg, about 34 mg, about 34.1 mg, about 34.2 mg, about 34.3 mg, about 34.4 mg, about 34.5 mg, about 34.6 mg, about 34.7 mg, about 34.8 mg, about 34.9 mg, about 35 mg, about 35.1 mg, about 35.2 mg, about 35.3 mg, about 35.4 mg, about 35.5 mg, about 35.6 mg, about 35.7 mg, about 35.8 mg, about 35.9 mg, about 36 mg, about 36.1 mg, about 36.2 mg, about 36.3 mg, about 36.4 mg, about 36.5 mg, about 36.6 mg, about 36.7 mg, about 36.8 mg, about 36.9 mg, about 37 mg, about 37.1 mg, about 37.2 mg, about 37.3 mg, about 37.4 mg, about 37.5 mg, about 37.6 mg, about 37.7 mg, about 37.8 mg, about 37.9 mg, about 38 mg, about 38.1 mg, about 38.2 mg, about 38.3 mg, about 38.4 mg, about 38.5 mg, about 38.6 mg, about 38.7 mg, about 38.8 mg, about 38.9 mg, about 39 mg, about 39.1 mg, about 39.2 mg, about 39.3 mg, about 39.4 mg, about 39.5 mg, about 39.6 mg, about 39.7 mg, about 39.8 mg, about 39.9 mg, and about 40 mg.

Embodiment 79. The method of any of embodiments 1-62, wherein the therapeutically effective amount is any of 40 mg, 40.1 mg, 40.2 mg, 40.3 mg, 40.4 mg, 40.5 mg, 40.6 mg, 40.7 mg, 40.8 mg, 40.9 mg, 41 mg, 41.1 mg, 41.2 mg, 41.3 mg, 41.4 mg, 41.5 mg, 41.6 mg, 41.7 mg, 41.8 mg, 41.9 mg, 42 mg, 42.1 mg, 42.2 mg, 42.3 mg, 42.4 mg, 42.5 mg, 42.6 mg, 42.7 mg, 42.8 mg, 42.9 mg, 43 mg, 43.1 mg, 43.2 mg, 43.3 mg, 43.4 mg, 43.5 mg, 43.6 mg, 43.7 mg, 43.8 mg, 43.9 mg, 44 mg, 44.1 mg, 44.2 mg, 44.3 mg, 44.4 mg, 44.5 mg, 44.6 mg, 44.7 mg, 44.8 mg, 44.9 mg, 45 mg, 45.1 mg, 45.2 mg, 45.3 mg, 45.4 mg, 45.5 mg, 45.6 mg, 45.7 mg, 45.8 mg, 45.9 mg, 46 mg, 46.1 mg, 46.2 mg, 46.3 mg, 46.4 mg, 46.5 mg, 46.6 mg, 46.7 mg, 46.8 mg, 46.9 mg, 47 mg, 47.1 mg, 47.2 mg, 47.3 mg, 47.4 mg, 47.5 mg, 47.6 mg, 47.7 mg, 47.8 mg, 47.9 mg, 48 mg, 48.1 mg, 48.2 mg, 48.3 mg, 48.4 mg, 48.5 mg, 48.6 mg, 48.7 mg, 48.8 mg, 48.9 mg, 49 mg, 49.1 mg, 49.2 mg, 49.3 mg, 49.4 mg, 49.5 mg, 49.6 mg, 49.7 mg, 49.8 mg, 49.9 mg, 50 mg, 50.1 mg, 50.2 mg, 50.3 mg, 50.4 mg, 50.5 mg, 50.6 mg, 50.7 mg, 50.8 mg, 50.9 mg, 51 mg, 51.1 mg, 51.2 mg, 51.3 mg, 51.4 mg, 51.5 mg, 51.6 mg, 51.7 mg, 51.8 mg, 51.9 mg, 52 mg, 52.1 mg, 52.2 mg, 52.3 mg, 52.4 mg, 52.5 mg, 52.6 mg, 52.7 mg, 52.8 mg, 52.9 mg, 53 mg, 53.1 mg, 53.2 mg, 53.3 mg, 53.4 mg, 53.5 mg, 53.6 mg, 53.7 mg, 53.8 mg, 53.9 mg, 54 mg, 54.1 mg, 54.2 mg, 54.3 mg, 54.4 mg, 54.5 mg, 54.6 mg, 54.7 mg, 54.8 mg, 54.9 mg, 55 mg, 55.1 mg, 55.2 mg, 55.3 mg, 55.4 mg, 55.5 mg, 55.6 mg, 55.7 mg, 55.8 mg, 55.9 mg, 56 mg, 56.1 mg, 56.2 mg, 56.3 mg, 56.4 mg, 56.5 mg, 56.6 mg, 56.7 mg, 56.8 mg, 56.9 mg, 57 mg, 57.1 mg, 57.2 mg, 57.3 mg, 57.4 mg, 57.5 mg, 57.6 mg, 57.7 mg, 57.8 mg, 57.9 mg, 58 mg, 58.1 mg, 58.2 mg, 58.3 mg, 58.4 mg, 58.5 mg, 58.6 mg, 58.7 mg, 58.8 mg, 58.9 mg, 59 mg, 59.1 mg, 59.2 mg, 59.3 mg, 59.4 mg, 59.5 mg, 59.6 mg, 59.7 mg, 59.8 mg, 59.9 mg, 60 mg, 60.1 mg, 60.2 mg, 60.3 mg, 60.4 mg, 60.5 mg, 60.6 mg, 60.7 mg, 60.8 mg, 60.9 mg, 61 mg, 61.1 mg, 61.2 mg, 61.3 mg, 61.4 mg, 61.5 mg, 61.6 mg, 61.7 mg, 61.8 mg, 61.9 mg, 62 mg, 62.1 mg, 62.2 mg, 62.3 mg, 62.4 mg, 62.5 mg, 62.6 mg, 62.7 mg, 62.8 mg, 62.9 mg, 63 mg, 63.1 mg, 63.2 mg, 63.3 mg, 63.4 mg, 63.5 mg, 63.6 mg, 63.7 mg, 63.8 mg, 63.9 mg, 64 mg, 64.1 mg, 64.2 mg, 64.3 mg, 64.4 mg, 64.5 mg, 64.6 mg, 64.7 mg, 64.8 mg, 64.9 mg, 65 mg, 65.1 mg, 65.2 mg, 65.3 mg, 65.4 mg, 65.5 mg, 65.6 mg, 65.7 mg, 65.8 mg, 65.9 mg, 66 mg, 66.1 mg, 66.2 mg, 66.3 mg, 66.4 mg, 66.5 mg, 66.6 mg, 66.7 mg, 66.8 mg, 66.9 mg, 67 mg, 67.1 mg, 67.2 mg, 67.3 mg, 67.4 mg, 67.5 mg, 67.6 mg, 67.7 mg, 67.8 mg, 67.9 mg, 68 mg, 68.1 mg, 68.2 mg, 68.3 mg, 68.4 mg, 68.5 mg, 68.6 mg, 68.7 mg, 68.8 mg, 68.9 mg, 69 mg, 69.1 mg, 69.2 mg, 69.3 mg, 69.4 mg, 69.5 mg, 69.6 mg, 69.7 mg, 69.8 mg, 69.9 mg, and 70 mg.

Embodiment 80. The method of any of embodiments 1-62, wherein the therapeutically effective amount is any of about 40 mg, about 40.1 mg, about 40.2 mg, about 40.3 mg, about 40.4 mg, about 40.5 mg, about 40.6 mg, about 40.7 mg, about 40.8 mg, about 40.9 mg, about 41 mg, about 41.1 mg, about 41.2 mg, about 41.3 mg, about 41.4 mg, about 41.5 mg, about 41.6 mg, about 41.7 mg, about 41.8 mg, about 41.9 mg, about 42 mg, about 42.1 mg, about 42.2 mg, about 42.3 mg, about 42.4 mg, about 42.5 mg, about 42.6 mg, about 42.7 mg, about 42.8 mg, about 42.9 mg, about 43 mg, about 43.1 mg, about 43.2 mg, about 43.3 mg, about 43.4 mg, about 43.5 mg, about 43.6 mg, about 43.7 mg, about 43.8 mg, about 43.9 mg, about 44 mg, about 44.1 mg, about 44.2 mg, about 44.3 mg, about 44.4 mg, about 44.5 mg, about 44.6 mg, about 44.7 mg, about 44.8 mg, about 44.9 mg, about 45 mg, about 45.1 mg, about 45.2 mg, about 45.3 mg, about 45.4 mg, about 45.5 mg, about 45.6 mg, about 45.7 mg, about 45.8 mg, about 45.9 mg, about 46 mg, about 46.1 mg, about 46.2 mg, about 46.3 mg, about 46.4 mg, about 46.5 mg, about 46.6 mg, about 46.7 mg, about 46.8 mg, about 46.9 mg, about 47 mg, about 47.1 mg, about 47.2 mg, about 47.3 mg, about 47.4 mg, about 47.5 mg, about 47.6 mg, about 47.7 mg, about 47.8 mg, about 47.9 mg, about 48 mg, about 48.1 mg, about 48.2 mg, about 48.3 mg, about 48.4 mg, about 48.5 mg, about 48.6 mg, about 48.7 mg, about 48.8 mg, about 48.9 mg, about 49 mg, about 49.1 mg, about 49.2 mg, about 49.3 mg, about 49.4 mg, about 49.5 mg, about 49.6 mg, about 49.7 mg, about 49.8 mg, about 49.9 mg, about 50 mg, about 50.1 mg, about 50.2 mg, about 50.3 mg, about 50.4 mg, about 50.5 mg, about 50.6 mg, about 50.7 mg, about 50.8 mg, about 50.9 mg, about 51 mg, about 51.1 mg, about 51.2 mg, about 51.3 mg, about 51.4 mg, about 51.5 mg, about 51.6 mg, about 51.7 mg, about 51.8 mg, about 51.9 mg, about 52 mg, about 52.1 mg, about 52.2 mg, about 52.3 mg, about 52.4 mg, about 52.5 mg, about 52.6 mg, about 52.7 mg, about 52.8 mg, about 52.9 mg, about 53 mg, about 53.1 mg, about 53.2 mg, about 53.3 mg, about 53.4 mg, about 53.5 mg, about 53.6 mg, about 53.7 mg, about 53.8 mg, about 53.9 mg, about 54 mg, about 54.1 mg, about 54.2 mg, about 54.3 mg, about 54.4 mg, about 54.5 mg, about 54.6 mg, about 54.7 mg, about 54.8 mg, about 54.9 mg, about 55 mg, about 55.1 mg, about 55.2 mg, about 55.3 mg, about 55.4 mg, about 55.5 mg, about 55.6 mg, about 55.7 mg, about 55.8 mg, about 55.9 mg, about 56 mg, about 56.1 mg, about 56.2 mg, about 56.3 mg, about 56.4 mg, about 56.5 mg, about 56.6 mg, about 56.7 mg, about 56.8 mg, about 56.9 mg, about 57 mg, about 57.1 mg, about 57.2 mg, about 57.3 mg, about 57.4 mg, about 57.5 mg, about 57.6 mg, about 57.7 mg, about 57.8 mg, about 57.9 mg, about 58 mg, about 58.1 mg, about 58.2 mg, about 58.3 mg, about 58.4 mg, about 58.5 mg, about 58.6 mg, about 58.7 mg, about 58.8 mg, about 58.9 mg, about 59 mg, about 59.1 mg, about 59.2 mg, about 59.3 mg, about 59.4 mg, about 59.5 mg, about 59.6 mg, about 59.7 mg, about 59.8 mg, about 59.9 mg, about 60 mg, about 60.1 mg, about 60.2 mg, about 60.3 mg, about 60.4 mg, about 60.5 mg, about 60.6 mg, about 60.7 mg, about 60.8 mg, about 60.9 mg, about 61 mg, about 61.1 mg, about 61.2 mg, about 61.3 mg, about 61.4 mg, about 61.5 mg, about 61.6 mg, about 61.7 mg, about 61.8 mg, about 61.9 mg, about 62 mg, about 62.1 mg, about 62.2 mg, about 62.3 mg, about 62.4 mg, about 62.5 mg, about 62.6 mg, about 62.7 mg, about 62.8 mg, about 62.9 mg, about 63 mg, about 63.1 mg, about 63.2 mg, about 63.3 mg, about 63.4 mg, about 63.5 mg, about 63.6 mg, about 63.7 mg, about 63.8 mg, about 63.9 mg, about 64 mg, about 64.1 mg, about 64.2 mg, about 64.3 mg, about 64.4 mg, about 64.5 mg, about 64.6 mg, about 64.7 mg, about 64.8 mg, about 64.9 mg, about 65 mg, about 65.1 mg, about 65.2 mg, about 65.3 mg, about 65.4 mg, about 65.5 mg, about 65.6 mg, about 65.7 mg, about 65.8 mg, about 65.9 mg, about 66 mg, about 66.1 mg, about 66.2 mg, about 66.3 mg, about 66.4 mg, about 66.5 mg, about 66.6 mg, about 66.7 mg, about 66.8 mg, about 66.9 mg, about 67 mg, about 67.1 mg, about 67.2 mg, about 67.3 mg, about 67.4 mg, about 67.5 mg, about 67.6 mg, about 67.7 mg, about 67.8 mg, about 67.9 mg, about 68 mg, about 68.1 mg, about 68.2 mg, about 68.3 mg, about 68.4 mg, about 68.5 mg, about 68.6 mg, about 68.7 mg, about 68.8 mg, about 68.9 mg, about 69 mg, about 69.1 mg, about 69.2 mg, about 69.3 mg, about 69.4 mg, about 69.5 mg, about 69.6 mg, about 69.7 mg, about 69.8 mg, about 69.9 mg, and about 70 mg.

Embodiment 81. The method of any of embodiments 1-62, wherein the therapeutically effective amount is any of 70 mg, 70.1 mg, 70.2 mg, 70.3 mg, 70.4 mg, 70.5 mg, 70.6 mg, 70.7 mg, 70.8 mg, 70.9 mg, 71 mg, 71.1 mg, 71.2 mg, 71.3 mg, 71.4 mg, 71.5 mg, 71.6 mg, 71.7 mg, 71.8 mg, 71.9 mg, 72 mg, 72.1 mg, 72.2 mg, 72.3 mg, 72.4 mg, 72.5 mg, 72.6 mg, 72.7 mg, 72.8 mg, 72.9 mg, 73 mg, 73.1 mg, 73.2 mg, 73.3 mg, 73.4 mg, 73.5 mg, 73.6 mg, 73.7 mg, 73.8 mg, 73.9 mg, 74 mg, 74.1 mg, 74.2 mg, 74.3 mg, 74.4 mg, 74.5 mg, 74.6 mg, 74.7 mg, 74.8 mg, 74.9 mg, 75 mg, 75.1 mg, 75.2 mg, 75.3 mg, 75.4 mg, 75.5 mg, 75.6 mg, 75.7 mg, 75.8 mg, 75.9 mg, 76 mg, 76.1 mg, 76.2 mg, 76.3 mg, 76.4 mg, 76.5 mg, 76.6 mg, 76.7 mg, 76.8 mg, 76.9 mg, 77 mg, 77.1 mg, 77.2 mg, 77.3 mg, 77.4 mg, 77.5 mg, 77.6 mg, 77.7 mg, 77.8 mg, 77.9 mg, 78 mg, 78.1 mg, 78.2 mg, 78.3 mg, 78.4 mg, 78.5 mg, 78.6 mg, 78.7 mg, 78.8 mg, 78.9 mg, 79 mg, 79.1 mg, 79.2 mg, 79.3 mg, 79.4 mg, 79.5 mg, 79.6 mg, 79.7 mg, 79.8 mg, 79.9 mg, 80 mg, 80.1 mg, 80.2 mg, 80.3 mg, 80.4 mg, 80.5 mg, 80.6 mg, 80.7 mg, 80.8 mg, 80.9 mg, 81 mg, 81.1 mg, 81.2 mg, 81.3 mg, 81.4 mg, 81.5 mg, 81.6 mg, 81.7 mg, 81.8 mg, 81.9 mg, 82 mg, 82.1 mg, 82.2 mg, 82.3 mg, 82.4 mg, 82.5 mg, 82.6 mg, 82.7 mg, 82.8 mg, 82.9 mg, 83 mg, 83.1 mg, 83.2 mg, 83.3 mg, 83.4 mg, 83.5 mg, 83.6 mg, 83.7 mg, 83.8 mg, 83.9 mg, 84 mg, 84.1 mg, 84.2 mg, 84.3 mg, 84.4 mg, 84.5 mg, 84.6 mg, 84.7 mg, 84.8 mg, 84.9 mg, 85 mg, 85.1 mg, 85.2 mg, 85.3 mg, 85.4 mg, 85.5 mg, 85.6 mg, 85.7 mg, 85.8 mg, 85.9 mg, 86 mg, 86.1 mg, 86.2 mg, 86.3 mg, 86.4 mg, 86.5 mg, 86.6 mg, 86.7 mg, 86.8 mg, 86.9 mg, 87 mg, 87.1 mg, 87.2 mg, 87.3 mg, 87.4 mg, 87.5 mg, 87.6 mg, 87.7 mg, 87.8 mg, 87.9 mg, 88 mg, 88.1 mg, 88.2 mg, 88.3 mg, 88.4 mg, 88.5 mg, 88.6 mg, 88.7 mg, 88.8 mg, 88.9 mg, 89 mg, 89.1 mg, 89.2 mg, 89.3 mg, 89.4 mg, 89.5 mg, 89.6 mg, 89.7 mg, 89.8 mg, 89.9 mg, 90 mg, 90.1 mg, 90.2 mg, 90.3 mg, 90.4 mg, 90.5 mg, 90.6 mg, 90.7 mg, 90.8 mg, 90.9 mg, 91 mg, 91.1 mg, 91.2 mg, 91.3 mg, 91.4 mg, 91.5 mg, 91.6 mg, 91.7 mg, 91.8 mg, 91.9 mg, 92 mg, 92.1 mg, 92.2 mg, 92.3 mg, 92.4 mg, 92.5 mg, 92.6 mg, 92.7 mg, 92.8 mg, 92.9 mg, 93 mg, 93.1 mg, 93.2 mg, 93.3 mg, 93.4 mg, 93.5 mg, 93.6 mg, 93.7 mg, 93.8 mg, 93.9 mg, 94 mg, 94.1 mg, 94.2 mg, 94.3 mg, 94.4 mg, 94.5 mg, 94.6 mg, 94.7 mg, 94.8 mg, 94.9 mg, 95 mg, 95.1 mg, 95.2 mg, 95.3 mg, 95.4 mg, 95.5 mg, 95.6 mg, 95.7 mg, 95.8 mg, 95.9 mg, 96 mg, 96.1 mg, 96.2 mg, 96.3 mg, 96.4 mg, 96.5 mg, 96.6 mg, 96.7 mg, 96.8 mg, 96.9 mg, 97 mg, 97.1 mg, 97.2 mg, 97.3 mg, 97.4 mg, 97.5 mg, 97.6 mg, 97.7 mg, 97.8 mg, 97.9 mg, 98 mg, 98.1 mg, 98.2 mg, 98.3 mg, 98.4 mg, 98.5 mg, 98.6 mg, 98.7 mg, 98.8 mg, 98.9 mg, 99 mg, 99.1 mg, 99.2 mg, 99.3 mg, 99.4 mg, 99.5 mg, 99.6 mg, 99.7 mg, 99.8 mg, and 99.9 mg.

Embodiment 82. The method of any of embodiments 1-62, wherein the therapeutically effective amount is any of about 70 mg, about 70.1 mg, about 70.2 mg, about 70.3 mg, about 70.4 mg, about 70.5 mg, about 70.6 mg, about 70.7 mg, about 70.8 mg, about 70.9 mg, about 71 mg, about 71.1 mg, about 71.2 mg, about 71.3 mg, about 71.4 mg, about 71.5 mg, about 71.6 mg, about 71.7 mg, about 71.8 mg, about 71.9 mg, about 72 mg, about 72.1 mg, about 72.2 mg, about 72.3 mg, about 72.4 mg, about 72.5 mg, about 72.6 mg, about 72.7 mg, about 72.8 mg, about 72.9 mg, about 73 mg, about 73.1 mg, about 73.2 mg, about 73.3 mg, about 73.4 mg, about 73.5 mg, about 73.6 mg, about 73.7 mg, about 73.8 mg, about 73.9 mg, about 74 mg, about 74.1 mg, about 74.2 mg, about 74.3 mg, about 74.4 mg, about 74.5 mg, about 74.6 mg, about 74.7 mg, about 74.8 mg, about 74.9 mg, about 75 mg, about 75.1 mg, about 75.2 mg, about 75.3 mg, about 75.4 mg, about 75.5 mg, about 75.6 mg, about 75.7 mg, about 75.8 mg, about 75.9 mg, about 76 mg, about 76.1 mg, about 76.2 mg, about 76.3 mg, about 76.4 mg, about 76.5 mg, about 76.6 mg, about 76.7 mg, about 76.8 mg, about 76.9 mg, about 77 mg, about 77.1 mg, about 77.2 mg, about 77.3 mg, about 77.4 mg, about 77.5 mg, about 77.6 mg, about 77.7 mg, about 77.8 mg, about 77.9 mg, about 78 mg, about 78.1 mg, about 78.2 mg, about 78.3 mg, about 78.4 mg, about 78.5 mg, about 78.6 mg, about 78.7 mg, about 78.8 mg, about 78.9 mg, about 79 mg, about 79.1 mg, about 79.2 mg, about 79.3 mg, about 79.4 mg, about 79.5 mg, about 79.6 mg, about 79.7 mg, about 79.8 mg, about 79.9 mg, about 80 mg, about 80.1 mg, about 80.2 mg, about 80.3 mg, about 80.4 mg, about 80.5 mg, about 80.6 mg, about 80.7 mg, about 80.8 mg, about 80.9 mg, about 81 mg, about 81.1 mg, about 81.2 mg, about 81.3 mg, about 81.4 mg, about 81.5 mg, about 81.6 mg, about 81.7 mg, about 81.8 mg, about 81.9 mg, about 82 mg, about 82.1 mg, about 82.2 mg, about 82.3 mg, about 82.4 mg, about 82.5 mg, about 82.6 mg, about 82.7 mg, about 82.8 mg, about 82.9 mg, about 83 mg, about 83.1 mg, about 83.2 mg, about 83.3 mg, about 83.4 mg, about 83.5 mg, about 83.6 mg, about 83.7 mg, about 83.8 mg, about 83.9 mg, about 84 mg, about 84.1 mg, about 84.2 mg, about 84.3 mg, about 84.4 mg, about 84.5 mg, about 84.6 mg, about 84.7 mg, about 84.8 mg, about 84.9 mg, about 85 mg, about 85.1 mg, about 85.2 mg, about 85.3 mg, about 85.4 mg, about 85.5 mg, about 85.6 mg, about 85.7 mg, about 85.8 mg, about 85.9 mg, about 86 mg, about 86.1 mg, about 86.2 mg, about 86.3 mg, about 86.4 mg, about 86.5 mg, about 86.6 mg, about 86.7 mg, about 86.8 mg, about 86.9 mg, about 87 mg, about 87.1 mg, about 87.2 mg, about 87.3 mg, about 87.4 mg, about 87.5 mg, about 87.6 mg, about 87.7 mg, about 87.8 mg, about 87.9 mg, about 88 mg, about 88.1 mg, about 88.2 mg, about 88.3 mg, about 88.4 mg, about 88.5 mg, about 88.6 mg, about 88.7 mg, about 88.8 mg, about 88.9 mg, about 89 mg, about 89.1 mg, about 89.2 mg, about 89.3 mg, about 89.4 mg, about 89.5 mg, about 89.6 mg, about 89.7 mg, about 89.8 mg, about 89.9 mg, about 90 mg, about 90.1 mg, about 90.2 mg, about 90.3 mg, about 90.4 mg, about 90.5 mg, about 90.6 mg, about 90.7 mg, about 90.8 mg, about 90.9 mg, about 91 mg, about 91.1 mg, about 91.2 mg, about 91.3 mg, about 91.4 mg, about 91.5 mg, about 91.6 mg, about 91.7 mg, about 91.8 mg, about 91.9 mg, about 92 mg, about 92.1 mg, about 92.2 mg, about 92.3 mg, about 92.4 mg, about 92.5 mg, about 92.6 mg, about 92.7 mg, about 92.8 mg, about 92.9 mg, about 93 mg, about 93.1 mg, about 93.2 mg, about 93.3 mg, about 93.4 mg, about 93.5 mg, about 93.6 mg, about 93.7 mg, about 93.8 mg, about 93.9 mg, about 94 mg, about 94.1 mg, about 94.2 mg, about 94.3 mg, about 94.4 mg, about 94.5 mg, about 94.6 mg, about 94.7 mg, about 94.8 mg, about 94.9 mg, about 95 mg, about 95.1 mg, about 95.2 mg, about 95.3 mg, about 95.4 mg, about 95.5 mg, about 95.6 mg, about 95.7 mg, about 95.8 mg, about 95.9 mg, about 96 mg, about 96.1 mg, about 96.2 mg, about 96.3 mg, about 96.4 mg, about 96.5 mg, about 96.6 mg, about 96.7 mg, about 96.8 mg, about 96.9 mg, about 97 mg, about 97.1 mg, about 97.2 mg, about 97.3 mg, about 97.4 mg, about 97.5 mg, about 97.6 mg, about 97.7 mg, about 97.8 mg, about 97.9 mg, about 98 mg, about 98.1 mg, about 98.2 mg, about 98.3 mg, about 98.4 mg, about 98.5 mg, about 98.6 mg, about 98.7 mg, about 98.8 mg, about 98.9 mg, about 99 mg, about 99.1 mg, about 99.2 mg, about 99.3 mg, about 99.4 mg, about 99.5 mg, about 99.6 mg, about 99.7 mg, about 99.8 mg, and about 99.9 mg.

Embodiment 83. The method of any of embodiments 1-62, wherein the therapeutically effective amount is any of 100 mg, 100.1 mg, 100.2 mg, 100.3 mg, 100.4 mg, 100.5 mg, 100.6 mg, 100.7 mg, 100.8 mg, 100.9 mg, 101 mg, 101.1 mg, 101.2 mg, 101.3 mg, 101.4 mg, 101.5 mg, 101.6 mg, 101.7 mg, 101.8 mg, 101.9 mg, 102 mg, 102.1 mg, 102.2 mg, 102.3 mg, 102.4 mg, 102.5 mg, 102.6 mg, 102.7 mg, 102.8 mg, 102.9 mg, 103 mg, 103.1 mg, 103.2 mg, 103.3 mg, 103.4 mg, 103.5 mg, 103.6 mg, 103.7 mg, 103.8 mg, 103.9 mg, 104 mg, 104.1 mg, 104.2 mg, 104.3 mg, 104.4 mg, 104.5 mg, 104.6 mg, 104.7 mg, 104.8 mg, 104.9 mg, 105 mg, 105.1 mg, 105.2 mg, 105.3 mg, 105.4 mg, 105.5 mg, 105.6 mg, 105.7 mg, 105.8 mg, 105.9 mg, 106 mg, 106.1 mg, 106.2 mg, 106.3 mg, 106.4 mg, 106.5 mg, 106.6 mg, 106.7 mg, 106.8 mg, 106.9 mg, 107 mg, 107.1 mg, 107.2 mg, 107.3 mg, 107.4 mg, 107.5 mg, 107.6 mg, 107.7 mg, 107.8 mg, 107.9 mg, 108 mg, 108.1 mg, 108.2 mg, 108.3 mg, 108.4 mg, 108.5 mg, 108.6 mg, 108.7 mg, 108.8 mg, 108.9 mg, 109 mg, 109.1 mg, 109.2 mg, 109.3 mg, 109.4 mg, 109.5 mg, 109.6 mg, 109.7 mg, 109.8 mg, 109.9 mg, 110 mg, 110.1 mg, 110.2 mg, 110.3 mg, 110.4 mg, 110.5 mg, 110.6 mg, 110.7 mg, 110.8 mg, 110.9 mg, 111 mg, 111.1 mg, 111.2 mg, 111.3 mg, 111.4 mg, 111.5 mg, 111.6 mg, 111.7 mg, 111.8 mg, 111.9 mg, 112 mg, 112.1 mg, 112.2 mg, 112.3 mg, 112.4 mg, 112.5 mg, 112.6 mg, 112.7 mg, 112.8 mg, 112.9 mg, 113 mg, 113.1 mg, 113.2 mg, 113.3 mg, 113.4 mg, 113.5 mg, 113.6 mg, 113.7 mg, 113.8 mg, 113.9 mg, 114 mg, 114.1 mg, 114.2 mg, 114.3 mg, 114.4 mg, 114.5 mg, 114.6 mg, 114.7 mg, 114.8 mg, 114.9 mg, 115 mg, 115.1 mg, 115.2 mg, 115.3 mg, 115.4 mg, 115.5 mg, 115.6 mg, 115.7 mg, 115.8 mg, 115.9 mg, 116 mg, 116.1 mg, 116.2 mg, 116.3 mg, 116.4 mg, 116.5 mg, 116.6 mg, 116.7 mg, 116.8 mg, 116.9 mg, 117 mg, 117.1 mg, 117.2 mg, 117.3 mg, 117.4 mg, 117.5 mg, 117.6 mg, 117.7 mg, 117.8 mg, 117.9 mg, 118 mg, 118.1 mg, 118.2 mg, 118.3 mg, 118.4 mg, 118.5 mg, 118.6 mg, 118.7 mg, 118.8 mg, 118.9 mg, 119 mg, 119.1 mg, 119.2 mg, 119.3 mg, 119.4 mg, 119.5 mg, 119.6 mg, 119.7 mg, 119.8 mg, 119.9 mg, 120 mg, 120.1 mg, 120.2 mg, 120.3 mg, 120.4 mg, 120.5 mg, 120.6 mg, 120.7 mg, 120.8 mg, 120.9 mg, 121 mg, 121.1 mg, 121.2 mg, 121.3 mg, 121.4 mg, 121.5 mg, 121.6 mg, 121.7 mg, 121.8 mg, 121.9 mg, 122 mg, 122.1 mg, 122.2 mg, 122.3 mg, 122.4 mg, 122.5 mg, 122.6 mg, 122.7 mg, 122.8 mg, 122.9 mg, 123 mg, 123.1 mg, 123.2 mg, 123.3 mg, 123.4 mg, 123.5 mg, 123.6 mg, 123.7 mg, 123.8 mg, 123.9 mg, 124 mg, 124.1 mg, 124.2 mg, 124.3 mg, 124.4 mg, 124.5 mg, 124.6 mg, 124.7 mg, 124.8 mg, 124.9 mg, and 125 mg.

Embodiment 84. The method of any of embodiments 1-62, wherein the therapeutically effective amount is any of about 100 mg, about 100.1 mg, about 100.2 mg, about 100.3 mg, about 100.4 mg, about 100.5 mg, about 100.6 mg, about 100.7 mg, about 100.8 mg, about 100.9 mg, about 101 mg, about 101.1 mg, about 101.2 mg, about 101.3 mg, about 101.4 mg, about 101.5 mg, about 101.6 mg, about 101.7 mg, about 101.8 mg, about 101.9 mg, about 102 mg, about 102.1 mg, about 102.2 mg, about 102.3 mg, about 102.4 mg, about 102.5 mg, about 102.6 mg, about 102.7 mg, about 102.8 mg, about 102.9 mg, about 103 mg, about 103.1 mg, about 103.2 mg, about 103.3 mg, about 103.4 mg, about 103.5 mg, about 103.6 mg, about 103.7 mg, about 103.8 mg, about 103.9 mg, about 104 mg, about 104.1 mg, about 104.2 mg, about 104.3 mg, about 104.4 mg, about 104.5 mg, about 104.6 mg, about 104.7 mg, about 104.8 mg, about 104.9 mg, about 105 mg, about 105.1 mg, about 105.2 mg, about 105.3 mg, about 105.4 mg, about 105.5 mg, about 105.6 mg, about 105.7 mg, about 105.8 mg, about 105.9 mg, about 106 mg, about 106.1 mg, about 106.2 mg, about 106.3 mg, about 106.4 mg, about 106.5 mg, about 106.6 mg, about 106.7 mg, about 106.8 mg, about 106.9 mg, about 107 mg, about 107.1 mg, about 107.2 mg, about 107.3 mg, about 107.4 mg, about 107.5 mg, about 107.6 mg, about 107.7 mg, about 107.8 mg, about 107.9 mg, about 108 mg, about 108.1 mg, about 108.2 mg, about 108.3 mg, about 108.4 mg, about 108.5 mg, about 108.6 mg, about 108.7 mg, about 108.8 mg, about 108.9 mg, about 109 mg, about 109.1 mg, about 109.2 mg, about 109.3 mg, about 109.4 mg, about 109.5 mg, about 109.6 mg, about 109.7 mg, about 109.8 mg, about 109.9 mg, about 110 mg, about 110.1 mg, about 110.2 mg, about 110.3 mg, about 110.4 mg, about 110.5 mg, about 110.6 mg, about 110.7 mg, about 110.8 mg, about 110.9 mg, about 111 mg, about 111.1 mg, about 111.2 mg, about 111.3 mg, about 111.4 mg, about 111.5 mg, about 111.6 mg, about 111.7 mg, about 111.8 mg, about 111.9 mg, about 112 mg, about 112.1 mg, about 112.2 mg, about 112.3 mg, about 112.4 mg, about 112.5 mg, about 112.6 mg, about 112.7 mg, about 112.8 mg, about 112.9 mg, about 113 mg, about 113.1 mg, about 113.2 mg, about 113.3 mg, about 113.4 mg, about 113.5 mg, about 113.6 mg, about 113.7 mg, about 113.8 mg, about 113.9 mg, about 114 mg, about 114.1 mg, about 114.2 mg, about 114.3 mg, about 114.4 mg, about 114.5 mg, about 114.6 mg, about 114.7 mg, about 114.8 mg, about 114.9 mg, about 115 mg, about 115.1 mg, about 115.2 mg, about 115.3 mg, about 115.4 mg, about 115.5 mg, about 115.6 mg, about 115.7 mg, about 115.8 mg, about 115.9 mg, about 116 mg, about 116.1 mg, about 116.2 mg, about 116.3 mg, about 116.4 mg, about 116.5 mg, about 116.6 mg, about 116.7 mg, about 116.8 mg, about 116.9 mg, about 117 mg, about 117.1 mg, about 117.2 mg, about 117.3 mg, about 117.4 mg, about 117.5 mg, about 117.6 mg, about 117.7 mg, about 117.8 mg, about 117.9 mg, about 118 mg, about 118.1 mg, about 118.2 mg, about 118.3 mg, about 118.4 mg, about 118.5 mg, about 118.6 mg, about 118.7 mg, about 118.8 mg, about 118.9 mg, about 119 mg, about 119.1 mg, about 119.2 mg, about 119.3 mg, about 119.4 mg, about 119.5 mg, about 119.6 mg, about 119.7 mg, about 119.8 mg, about 119.9 mg, about 120 mg, about 120.1 mg, about 120.2 mg, about 120.3 mg, about 120.4 mg, about 120.5 mg, about 120.6 mg, about 120.7 mg, about 120.8 mg, about 120.9 mg, about 121 mg, about 121.1 mg, about 121.2 mg, about 121.3 mg, about 121.4 mg, about 121.5 mg, about 121.6 mg, about 121.7 mg, about 121.8 mg, about 121.9 mg, about 122 mg, about 122.1 mg, about 122.2 mg, about 122.3 mg, about 122.4 mg, about 122.5 mg, about 122.6 mg, about 122.7 mg, about 122.8 mg, about 122.9 mg, about 123 mg, about 123.1 mg, about 123.2 mg, about 123.3 mg, about 123.4 mg, about 123.5 mg, about 123.6 mg, about 123.7 mg, about 123.8 mg, about 123.9 mg, about 124 mg, about 124.1 mg, about 124.2 mg, about 124.3 mg, about 124.4 mg, about 124.5 mg, about 124.6 mg, about 124.7 mg, about 124.8 mg, about 124.9 mg, and about 125 mg.

Embodiment 85. The method of any of embodiments 1-62, wherein the therapeutically effective amount is within the range of any of 10 mg to 200 mg, 10 mg to 190 mg, 10 mg to 180 mg, 10 mg to 170 mg, from 10 mg to 160 mg, 10 mg to 150 mg, 10 mg to 140 mg, 10 mg to 120 mg, 10 mg to 110 mg, 10 mg to 100 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 200 mg, 20 mg to 190 mg, 20 mg to 180 mg, 20 mg to 170 mg, from 20 mg to 160 mg, 20 mg to 150 mg, 20 mg to 140 mg, 20 mg to 120 mg, 20 mg to 110 mg, 20 mg to 100 mg, 20 mg to 80 mg, 20 mg to 70 mg, 20 mg to 60 mg, 20 mg to 50 mg, 20 mg to 10 mg, 20 mg to 30 mg, 30 mg to 200 mg, 30 mg to 190 mg, 30 mg to 180 mg, 30 mg to 170 mg, from 30 mg to 160 mg, 30 mg to 150 mg, 30 mg to 140 mg, 30 mg to 120 mg, 30 mg to 110 mg, 30 mg to 100 mg, 30 mg to 80 mg, 30 mg to 70 mg, 30 mg to 60 mg, 30 mg to 50 mg, 30 mg to 20 mg, 40 mg to 200 mg, 40 mg to 190 mg, 40 mg to 180 mg, 40 mg to 170 mg, from 40 mg to 160 mg, 40 mg to 150 mg, 40 mg to 140 mg, 40 mg to 120 mg, 40 mg to 110 mg, 40 mg to 100 mg, 40 mg to 80 mg, 40 mg to 70 mg, 40 mg to 60 mg, 40 mg to 50 mg, 50 mg to 200 mg, 50 mg to 190 mg, 50 mg to 180 mg, 50 mg to 170 mg, 50 mg to 160 mg, 50 mg to 150 mg, 50 mg to 140 mg, 50 mg to 120 mg, 50 mg to 110 mg, 50 mg to 100 mg, 50 mg to 80 mg, 50 mg to 70 mg, 50 mg to 60 mg, 60 mg to 200 mg, 60 mg to 190 mg, 60 mg to 180 mg, 60 mg to 170 mg, 60 mg to 160 mg, 60 mg to 150 mg, 60 mg to 140 mg, 60 mg to 120 mg, 60 mg to 110 mg, 60 mg to 100 mg, 60 mg to 80 mg, 60 mg to 70 mg, 70 mg to 200 mg, 70 mg to 190 mg, 70 mg to 180 mg, 70 mg to 170 mg, 70 mg to 160 mg, 70 mg to 150 mg, 70 mg to 140 mg, 70 mg to 120 mg, 70 mg to 110 mg, 70 mg to 100 mg, 70 mg to 80 mg, 80 mg to 200 mg, 80 mg to 190 mg, 80 mg to 180 mg, 80 mg to 170 mg, 80 mg to 160 mg, 80 mg to 150 mg, 80 mg to 140 mg, 80 mg to 120 mg, 80 mg to 110 mg, 80 mg to 100 mg, 80 mg to 90 mg, 90 mg to 200 mg, 90 mg to 190 mg, 90 mg to 180 mg, 90 mg to 170 mg, 90 mg to 160 mg, 90 mg to 150 mg, 90 mg to 140 mg, 90 mg to 120 mg, 90 mg to 110 mg, 90 mg to 100 mg, 100 mg to 200 mg, 100 mg to 190 mg, 100 mg to 180 mg, 100 mg to 170 mg, 100 mg to 160 mg, 100 mg to 150 mg, 100 mg to 140 mg, 100 mg to 120 mg, 100 mg to 110 mg, 110 mg to 200 mg, 110 mg to 190 mg, 110 mg to 180 mg, 110 mg to 170 mg, 110 mg to 160 mg, 110 mg to 150 mg, 110 mg to 140 mg, 110 mg to 130 mg, 110 mg to 120 mg, 120 mg to 200 mg, 120 mg to 190 mg, 120 mg to 180 mg, 120 mg to 170 mg, 120 mg to 160 mg, 120 mg to 150 mg, 120 mg to 140 mg, 120 mg to 130 mg, 130 mg to 200 mg, 130 mg to 190 mg, 130 mg to 180 mg, 130 mg to 170 mg, 130 mg to 160 mg, 130 mg to 150 mg, 130 mg to 140 mg, 140 mg to 200 mg, 140 mg to 190 mg, 140 mg to 180 mg, 140 mg to 170 mg, 140 mg to 160 mg, 140 mg to 150 mg, 150 mg to 200 mg, 150 mg to 190 mg, 150 mg to 180 mg, 150 mg to 170 mg, 150 mg to 160 mg, 160 mg to 200 mg, 160 mg to 190 mg, 160 mg to 180 mg, 160 mg to 170 mg, 180 mg to 200 mg, 180 mg to 190 mg, 190 mg to 200 mg, 105 mg to 135 mg, 105 mg to 130 mg, 105 mg to 125 mg 105 mg to 120 mg, 110 mg to 135 mg, 110 mg to 130 mg, 110 mg to 125 mg, 110 mg to 120 mg, 115 mg to 135 mg, 115 mg to 130 mg, 115 mg to 125 mg, 115 mg to 120 mg, 115 mg to 125 mg, 115 mg to 120 mg, 120 mg to 135 mg, 120 mg to 125 mg, 125 mg to 140 mg, 125 mg to 130 mg, 130 mg to 135 mg, 135 mg to 140 mg, 120 mg to 129 mg, 120 mg to 128 mg, 120 mg to 127 mg, 120 mg to 86 mg, 120 mg to 124 mg, 120 mg to 123 mg, 120 mg to 122 mg, 120 mg to 121 mg, 121 mg to 130 mg, 122 mg to 129 mg, 122 mg to 128 mg, 122 mg to 127 mg, 122 mg to 126 mg, 122 mg to 125 mg, 122 mg to 124 mg, 122 mg to 123 mg, 123 mg to 130 mg, 123 mg to 129 mg, 123 mg to 128 mg, 123 mg to 127 mg, 123 mg to 126 mg, 123 mg to 125 mg, 123 mg to 124 mg, 124 mg to 130 mg, 124 mg to 129 mg, 124 mg to 128 mg, 124 mg to 127 mg, 124 mg to 126 mg, 124 mg to 125 mg, 125 mg to 129 mg, 125 mg to 128 mg, 125 mg to 127 mg, 125 mg to 126 mg, 126 mg to 130 mg, 126 mg to 129 mg, 126 mg to 128 mg, 126 mg to 127 mg, 127 mg to 130 mg, 127 mg to 129 mg, 127 mg to 128 mg, 128 mg to 130 mg, 128 mg to 129 mg, and 129 mg to 130 mg.

Embodiment 86. The method of any of embodiments 1-62, wherein the therapeutically effective amount is from 1 mg to any of less than 350 mg, less than 345 mg, less than 340 mg, less than 335 mg, less than 330 mg, less than 325 mg, less than 320 mg, less than 315 mg, less than 310 mg, less than 305 mg, less than 300 mg, less than 295 mg, less than 290 mg, less than 285 mg, less than 280 mg, less than 275 mg, less than 270 mg, less than 265 mg, less than 260 mg, less than 255 mg, less than 250 mg, less than 245 mg, less than 240 mg, less than 235 mg, less than 230 mg, less than 225 mg, less than 220 mg, less than 215 mg, less than 210 mg, less than 205 mg, less than 200 mg, less than 195 mg, less than 190 mg, less than 185 mg, less than 180 mg, less than 175 mg, less than 170 mg, less than 165 mg, less than 160 mg, less than 150 mg, less than 145 mg, less than 140 mg, less than 135 mg, less than 130 mg, less than 125 mg, less than 120 mg, less than 115 mg, less than 110 mg, less than 105 mg, less than 100 mg, less than 95 mg, less than 90 mg, less than 85 mg, less than 80 mg, less than 75 mg, less than 70 mg, less than 65 mg, less than 60 mg, less than 55 mg, less than 50 mg, less than 45 mg, less than 40 mg, less than 35 mg, less than 30 mg, less than 25 mg, less than 20 mg, less than 15 mg, less than 10 mg, and less than 5 mg.

Embodiment 87. The method of any of embodiments 1-62, wherein the therapeutically effective amount is from 1 mg to any of less than about less than about 350 mg, less than about 345 mg, less than about 340 mg, less than about 335 mg, less than about 330 mg, less than about 325 mg, less than about 320 mg, less than about 315 mg, less than about 310 mg, less than about 305 mg, less than about 300 mg, less than about 295 mg, less than about 290 mg, less than about 285 mg, less than about 280 mg, less than about 275 mg, less than about 270 mg, less than about 265 mg, less than about 260 mg, less than about 255 mg, less than about 250 mg, less than about 245 mg, less than about 240 mg, less than about 235 mg, less than about 230 mg, less than about 225 mg, less than about 220 mg, less than about 215 mg, less than about 210 mg, less than about 205 mg, less than about 200 mg, less than about 195 mg, less than about 190 mg, less than about 185 mg, less than about 180 mg, less than about 175 mg, less than about 170 mg, less than about 165 mg, less than about 160 mg, less than about 150 mg, less than about 145 mg, less than about 140 mg, less than about 135 mg, less than about 130 mg, less than about 125 mg, less than about 120 mg, less than about 115 mg, less than about 110 mg, less than about 105 mg, less than about 100 mg, less than about 95 mg, less than about 90 mg, less than about 85 mg, less than about 80 mg, less than about 75 mg, less than about 70 mg, less than about 65 mg, less than about 60 mg, less than about 55 mg, less than about 50 mg, less than about 45 mg, less than about 40 mg, less than about 35 mg, less than about 30 mg, less than about 25 mg, less than about 20 mg, less than about 15 mg, less than about 10 mg, and less than about 5 mg.

Embodiment 88. The method of any of embodiments 1-62, wherein the therapeutically effective amount is any of at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least about 100 mg, at least 105 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185, at least 190 mg, at least 195 mg, and at least 200 mg.

Embodiment 89 The method of any of embodiments 1-62, wherein the therapeutically effective amount is from 1 mg to any of at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 115 mg, at least about 120 mg, at least about 125 mg, at least about 130 mg, at least about 135 mg, at least about 140 mg, at least about 145 mg, or at least about 150 mg, at least about 155 mg, at least about 160 mg, at least about 165 mg, at least about 170 mg, at least about 175 mg, at least about 180 mg, at least about 185, at least about 190 mg, at least about 195 mg, and at least about 200 mg.

Embodiment 90. The method of any of embodiments 1-89, comprising administering the compound once every 2 weeks.

Embodiment 91. The method of any of embodiments 1-89, comprising administering the compound once every 4 weeks.

Embodiment 92. The method of any of embodiments 1-89, comprising administering the compound once every 8 weeks.

Embodiment 93. The method of any of embodiments 1-89, comprising administering the compound once every 12 weeks.

Embodiment 94. The method of any of embodiments 1-89, comprising administering the compound once every 16 weeks.

Embodiment 95. The method of any of embodiments 1-89, comprising administering the compound once every 20 weeks.

Embodiment 96. The method of any of embodiments 1-89, comprising administering the compound once every 24 weeks.

Embodiment 97. The method of any of embodiments 1-89, comprising administering the compound once every 6 months.

Embodiment 98. The method of any of embodiments 1-89, comprising administering the compound about once every 2 weeks.

Embodiment 99. The method of any of embodiments 1-89, comprising administering the compound about once every 4 weeks.

Embodiment 100. The method of any of embodiments 1-89, comprising administering the compound about once every 8 weeks.

Embodiment 101. The method of any of embodiments 1-89, comprising administering the compound about once every 12 weeks.

Embodiment 102. The method of any of embodiments 1-89, comprising administering the compound about once every 16 weeks.

Embodiment 103. The method of any of embodiments 1-89, comprising administering the compound about once every 20 weeks.

Embodiment 104. The method of any of embodiments 1-89, comprising administering the compound about once every 24 weeks.

Embodiment 105. The method of any of embodiments 1-89, comprising administering the compound once about every 6 months.

Embodiment 106. The method of any of embodiments 1-89, comprising administering the compound once about every 6 months, once about every 7 months, once about every 8 months, once about every 9 months, once about every 10 months, once about every 11 months, or once about every 12 months.

Embodiment 107. The method of any of embodiments 1-89, comprising administering the compound monthly.

Embodiment 108. The method of any of embodiments 1-89, comprising administering the compound once every two months.

Embodiment 109. The method of any of embodiments 1-89, comprising administering the compound once every three months.

Embodiment 110. The method of any of embodiments 1-89, comprising administering the compound quarterly.

Embodiment 111. The method of any of embodiments 1-89, comprising administering the compound semiannually.

Embodiment 112. The method of any of embodiments 1-89, comprising administering the compound annually.

Embodiment 113. The method of any of embodiments 1-89, comprising administering the compound once every two years.

Embodiment 114. The method of any of embodiments 1-89, comprising administering the compound any of once every 1 week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every month, once every 2 months, once every 3 months, once every 4 months, once every 5 months, once every 6 months, once every 7 months, once every 8 months, once every 9 months, once every 10 months, once every 11 months, or once every year.

Embodiment 115. The method of any of embodiments 1-89, comprising administering the compound any of once about every 1 week, once about every 2 weeks, once about every 3 weeks, once about every 4 weeks, once about every 5 weeks, once about every 6 weeks, once about every 7 weeks, once about every 8 weeks, once about every 9 weeks, once about every 10 weeks, once about every 11 weeks, once about every 12 weeks, once about every 13 weeks, once about every 14 weeks, once about every 15 weeks, once about every 16 weeks, once about every 17 weeks, once about every 18 weeks, once about every 19 weeks, once about every 21 weeks, once about every 22 weeks, once about every 23 weeks, once about every 24 weeks, once about every month, once about every 2 months, once about every 3 months, once about every 4 months, once about every 5 months, and once about every 6 months, once about every 7 months, once about every 8 months, once about every 9 months, once about every 10 months, once about every 11 months, or once about every year.

Embodiment 116. The method of any of embodiments 1-89 comprising administering to the human subject a dose of 20 mg of the compound once every four weeks.

Embodiment 117. The method of any of embodiments 1-89 comprising administering to the human subject a dose of 40 mg of the compound once every four weeks.

Embodiment 118. The method of any of embodiments 1-89 comprising administering to the human subject a dose of 70 mg of the compound once every four weeks.

Embodiment 119. The method of any of embodiments 1-89 comprising administering to the human subject a dose of 100 mg of the compound once every 4 weeks.

Embodiment 120. The method of any of embodiments 1-89 comprising administering to the human subject a dose of 20 mg of the compound once every 8 weeks.

Embodiment 121. The method of any of embodiments 1-89 comprising administering to the human subject a dose of 40 mg of the compound once every 8 weeks.

Embodiment 122. The method of any of embodiments 1-89 comprising administering to the human subject a dose of 70 mg of the compound once every 8 weeks.

Embodiment 123. The method of any of embodiments 1-89 comprising administering to the human subject a dose of 100 mg of the compound once every 8 weeks.

Embodiment 124. The method of any of embodiments 1-89 comprising administering to the human subject a dose of 20 mg of the compound once every 12 weeks, once every 3 months, or once a quarter.

Embodiment 125. The method of any of embodiments 1-89 comprising administering to the human subject a dose of 40 mg of the compound once every 12 weeks, once every 3 months, or once a quarter.

Embodiment 126. The method of any of embodiments 1-89 comprising administering to the human subject a dose of 70 mg of the compound once every 12 weeks, once every 3 months, or once a quarter.

Embodiment 127. The method of any of embodiments 1-89 comprising administering to the human subject a dose of 100 mg of the compound once every 12 weeks, once every 3 months, or once a quarter.

Embodiment 128. The method of any of embodiments 1-89 comprising administering to the human subject a dose of 20 mg of the compound once every 6 months or twice a year.

Embodiment 129. The method of any of embodiments 1-89 comprising administering to the human subject a dose of 40 mg of the compound once every 6 months or twice a year.

Embodiment 130. The method of any of embodiments 1-89 comprising administering to the human subject a dose of 70 mg of the compound once every 6 months or twice a year.

Embodiment 131. The method of any of embodiments 1-89 comprising administering to the human subject a dose of 100 mg of the compound once every 6 months or twice a year.

Embodiment 132. The method of any of embodiments 1-131 wherein 2 doses of the compound are administered.

Embodiment 133. The method of any of embodiments 1-131, wherein 4 doses of the compound are administered.

Embodiment 134. The method of any of embodiments 1-89 comprising administering to the human subject a dose of 100 mg of the compound once every 2 weeks for total of 3 doses, then administering a dose of 100 mg of the compound once every 4 weeks thereafter.

Embodiment 135. The method of any of embodiments 1-89 comprising administering to the human subject a dose of 70 mg of the compound once every 2 weeks for total of 3 doses, then administering a dose of 70 mg of the compound once every 4 weeks thereafter.

Embodiment 136. The method of any of embodiments 1-89 comprising administering to the human subject a dose of 40 mg of the compound once every 2 weeks for total of 3 doses, then administering a dose of 40 mg of the compound once every 4 weeks thereafter.

Embodiment 137. The method of any of embodiments 1-89 comprising administering to the human subject a dose of 40 mg to 70 mg of the compound once every 4 weeks.

Embodiment 138. The method of any of embodiments 1-89 comprising administering to the human subject a dose of about 40 mg to about 70 mg of the compound once every 4 weeks.

Embodiment 139. The method of any of embodiments 1-89 comprising administering to the human subject a dose of 40 to 70 mg of the compound once every 2 weeks for total of 3 doses, then administering a dose of 40 mg to 70 mg of the compound once every 4 weeks thereafter.

Embodiment 140. The method of any of embodiments 1-89 comprising administering to the human subject a dose of about 40 mg to about 70 mg of the compound once every 2 weeks for total of 3 doses, then administering a dose of about 40 mg to about 70 mg of the compound once every 4 weeks thereafter.

Embodiment 141. The method of embodiment 139 or embodiment 140 wherein the same amount of compound is administered for the first 6 doses.

Embodiment 142. The method of any of embodiments 1-89 comprising administering to the human subject a dose of 70 mg of the compound once every 4 weeks for total of 3 doses, then administering a dose of 40 mg of the compound once every 4 weeks thereafter.

Embodiment 143. The method of any of embodiments 1-89 comprising administering to the human subject a dose of 70 mg of the compound once every 4 weeks for total of 3 doses, then administering a dose of 70 mg of the compound once every 8 weeks thereafter.

Embodiment 144. The method of any of embodiments 1-89, wherein the human subject has IgA Nephropathy, comprising administering to the subject a first dosing regimen comprising administering a dose of 100 mg of the compound once every two weeks for a total of 3 doses, then once every 4 weeks.

Embodiment 145. The method of embodiment 128, further comprising monitoring safety or efficacy of the dosing regimen, ceasing the administration of the first dosing regimen, then administering to the subject a second dosing regimen comprising administering a dose of 70 mg of the compound every 4 weeks.

Embodiment 146. The method of any of embodiments 90-145, wherein 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 doses are administered.

Embodiment 147. The method of any of embodiments 1-89 comprising administering to the human subject an initial loading dose of 70 mg of the compound.

Embodiment 148. The method of embodiment 147, comprising administering to the human subject a second loading dose of 70 mg of the compound 4 weeks after the initial loading dose. Embodiment 149. The method of embodiment 147 or embodiment 148, comprising administering to the human subject a maintenance dose of 40 mg of the compound 4 weeks after the second loading dose.

Embodiment 150. The method of embodiment 147 or embodiment 148, comprising administering to the human subject a maintenance dose of 40 mg of the compound 8 weeks after the second loading dose.

Embodiment 151. The method of embodiment 147 or embodiment 148, comprising administering to the human subject a maintenance dose of 40 mg of the compound 12 weeks after the second loading dose.

Embodiment 152. The method of claim 147 or embodiment 148, comprising administering to the human subject a maintenance dose of 10 mg, 20 mg, 40 mg, 70 mg, or 100 mg of the compound 4 weeks after the second loading dose and every 4 weeks thereafter.

Embodiment 153. The method of claim 147 or embodiment 148, comprising administering to the human subject a maintenance dose of 10 mg, 20 mg, 40 mg, 70 mg, or 100 mg of the compound 8 weeks after the second loading dose and every 8 weeks thereafter.

Embodiment 154. The method of claim 147 or embodiment 148, comprising administering to the human subject a maintenance dose of 10 mg, 20 mg, 40 mg, 70 mg, or 100 mg of the compound 12 weeks after the second loading dose and every 12 weeks thereafter.

Embodiment 155. The method of claim 147 or embodiment 148, comprising administering to the human subject a maintenance dose of 10 mg, 20 mg, 40 mg, 70 mg, or 100 mg of the compound 16 weeks after the second loading dose and every 16 weeks thereafter.

Embodiment 156. The method of claim 147 or embodiment 148, comprising administering to the human subject a maintenance dose of 10 mg, 20 mg, 40 mg, 70 mg, or 100 mg of the compound 24 weeks after the second loading dose and every 24 weeks thereafter.

Embodiment 157. The method of any of embodiments 149-156, wherein at least 2, at least 3, at least 4, at least 5, or at least 6 maintenance doses are administered to the human subject.

Embodiment 158. The method of any of embodiments 1-157, wherein the compound is administered subcutaneously.

Embodiment 159. The method of embodiment 145, wherein the compound is administered by injection.

Embodiment 160. The method of any of embodiments 1-159, wherein the compound is formulated as a pharmaceutical composition comprising the compound and a pharmaceutically acceptable diluent.

Embodiment 161. The method of embodiment 160, wherein the pharmaceutically acceptable diluent is sterile water, sterile saline, or sterile phosphate buffered saline.

Embodiment 162. The method of any of embodiments 1-161, wherein the compound is co-administered with an angiotensin converting enzyme inhibitor, an angiotensin receptor blocker, a sodium/glucose cotransporter-2 inhibitor, an anti-complement agent, or a complement inhibitor.

Embodiment 163. A method of ameliorating IgA Nephropathy, ameliorating Geographic Atrophy, reducing CFB protein, or reducing CFB RNA in a human subject in need thereof, the method comprising subcutaneously administering to the human subject 40 mg, about 40 mg, 70 mg, or about 70 mg of a compound according to the following chemical structure:

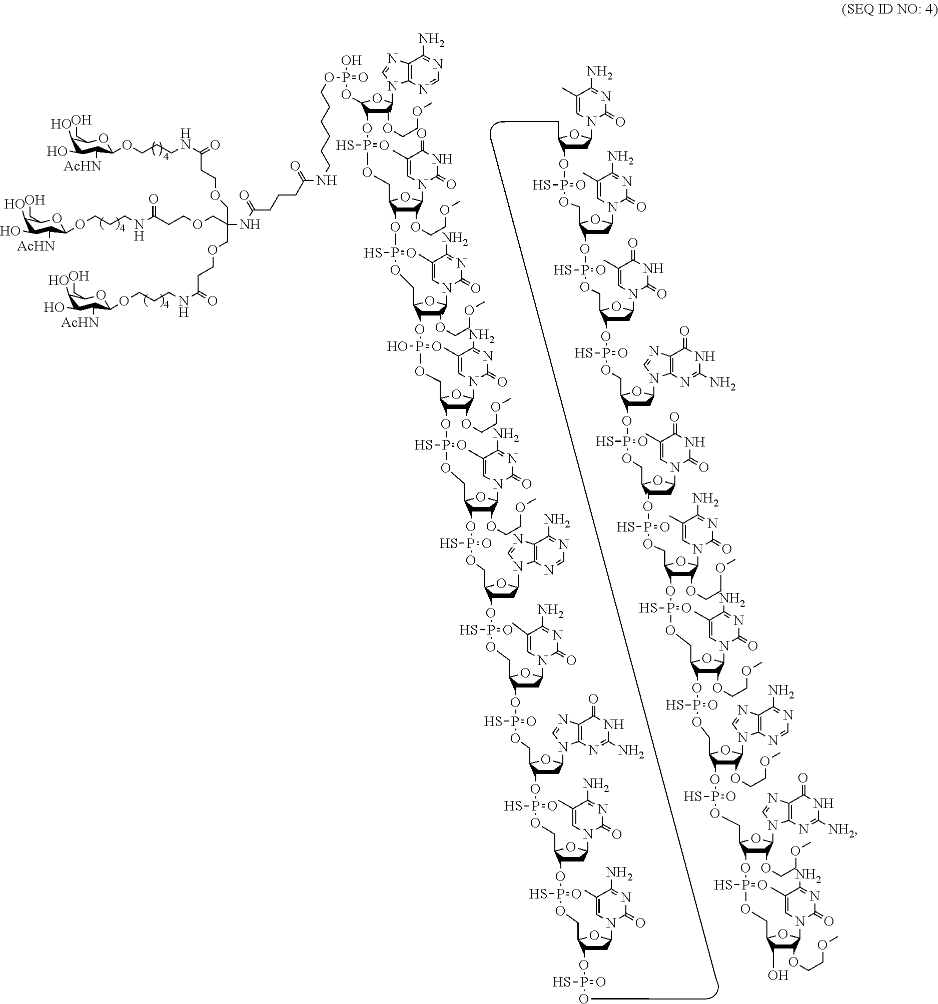

or a pharmaceutically acceptable salt thereof.

Embodiment 164. The method of embodiment 163, wherein the compound is the sodium salt or the potassium salt.

Embodiment 165. A method of ameliorating IgA Nephropathy, ameliorating Geographic Atrophy, reducing CFB protein, or reducing CFB RNA in a human subject in need thereof, the method comprising subcutaneously administering to the human subject 40 mg, about 40 mg, 70 mg, or about 70 mg of a compound according to the following chemical structure:

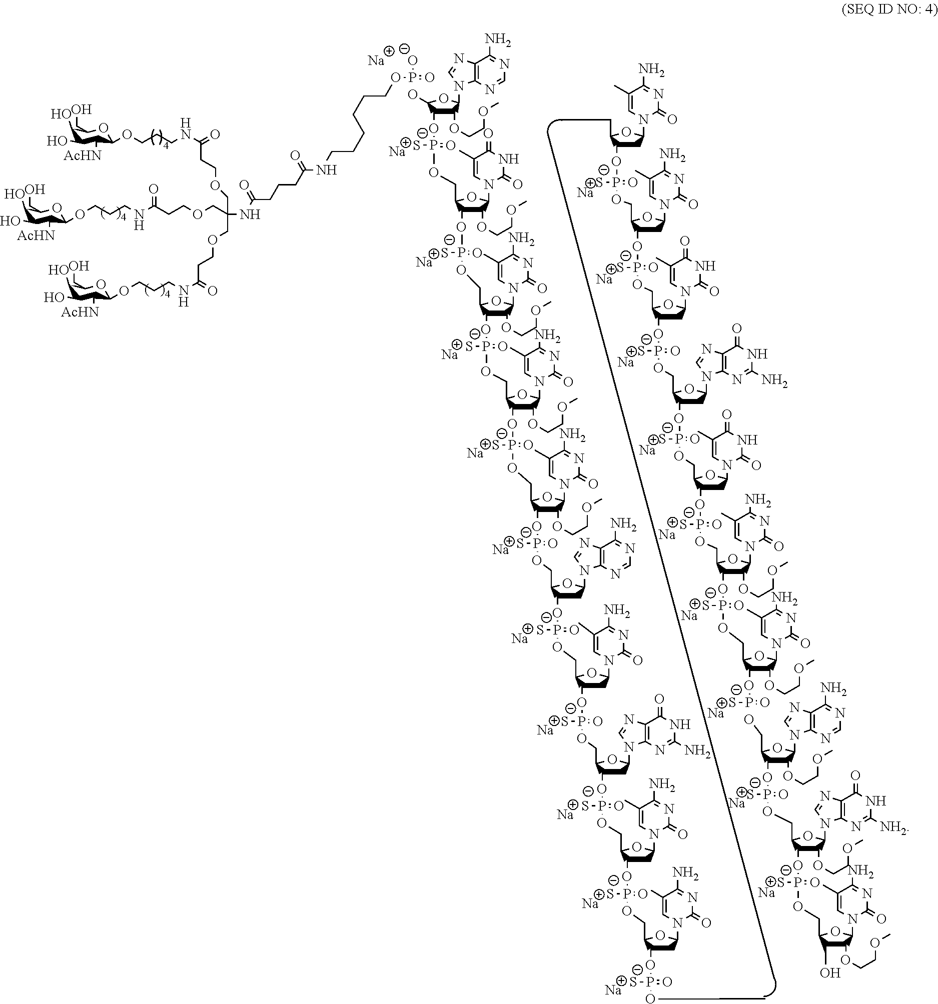

Embodiment 166. A method of ameliorating IgA Nephropathy, ameliorating Geographic Atrophy, reducing CFB protein, or reducing CFB RNA in a human subject in need thereof, the method comprising subcutaneously administering to the human subject 40 mg, about 40 mg, 70 mg, or about 70 mg of a compound, wherein the compound has the following chemical notation (5′ to 3′): GalNAc₃-7_{a-o′}
Aₑₛ Tₑₛ^{m}Cₑₛ^{m}Cₑₛ^{m}CₑₛA_{ds}^{m}C_{ds}G_{ds}^{m}C_{ds}^{m}C_{ds}^{m}C_{ds}^{m}C_{ds} T_{ds}G_{ds} T_{ds}^{m}Cₑₛ^{m}CₑₛAₑₛGₑₛ^{m}Cₑ (SEQ ID NO: 4); wherein,
   A=an adenine nucleobase,
   ^{m}C=a 5-methylcytosine nucleobase,
   G=a guanine nucleobase,
   T=a thymine nucleobase,
   e=a 2′-MOE sugar moiety,
   d=a 2′-β-D-deoxyribosyl sugar moiety,
   s=a phosphorothioate internucleoside linkage, and

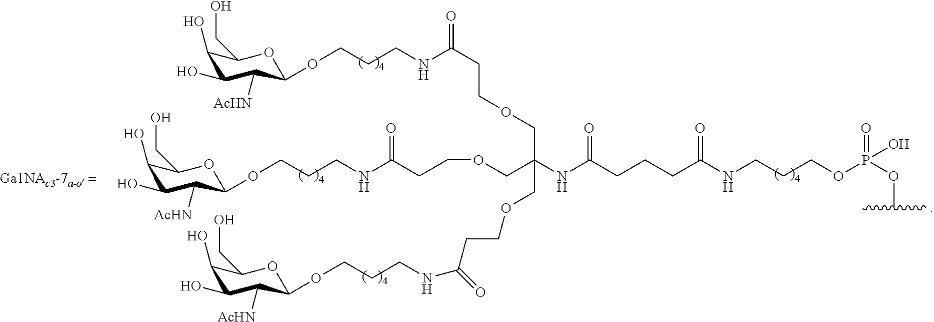

Embodiment 167. A method of ameliorating IgA Nephropathy in a human subject in need thereof, the method comprising subcutaneously administering to the human subject 40 mg, about 40 mg, 70 mg, or about 70 mg of a compound according to the following chemical structure:

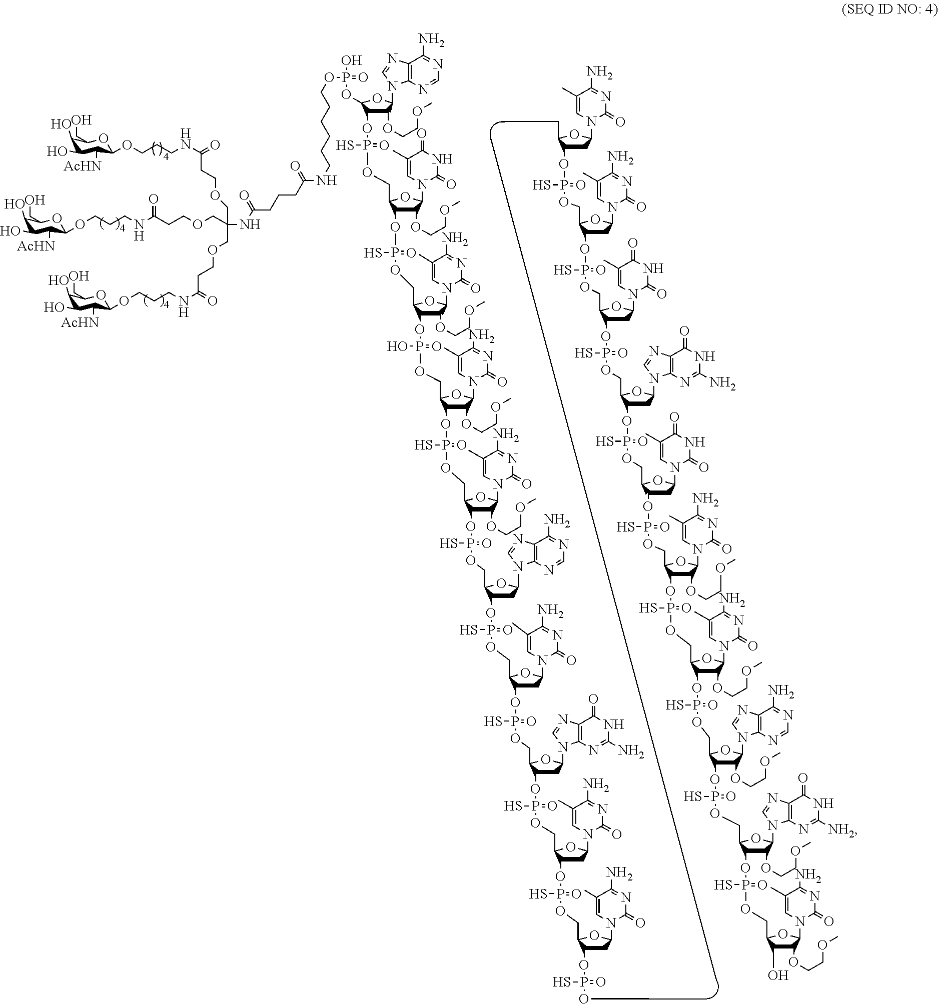

or a pharmaceutically acceptable salt thereof.

Embodiment 168. The method of embodiment 167, wherein the compound is the sodium salt or the potassium salt.

Embodiment 169. A method of ameliorating IgA Nephropathy in a human subject in need thereof, the method comprising subcutaneously administering to the human subject 70 mg, or about 70 mg of a compound according to the following chemical structure:

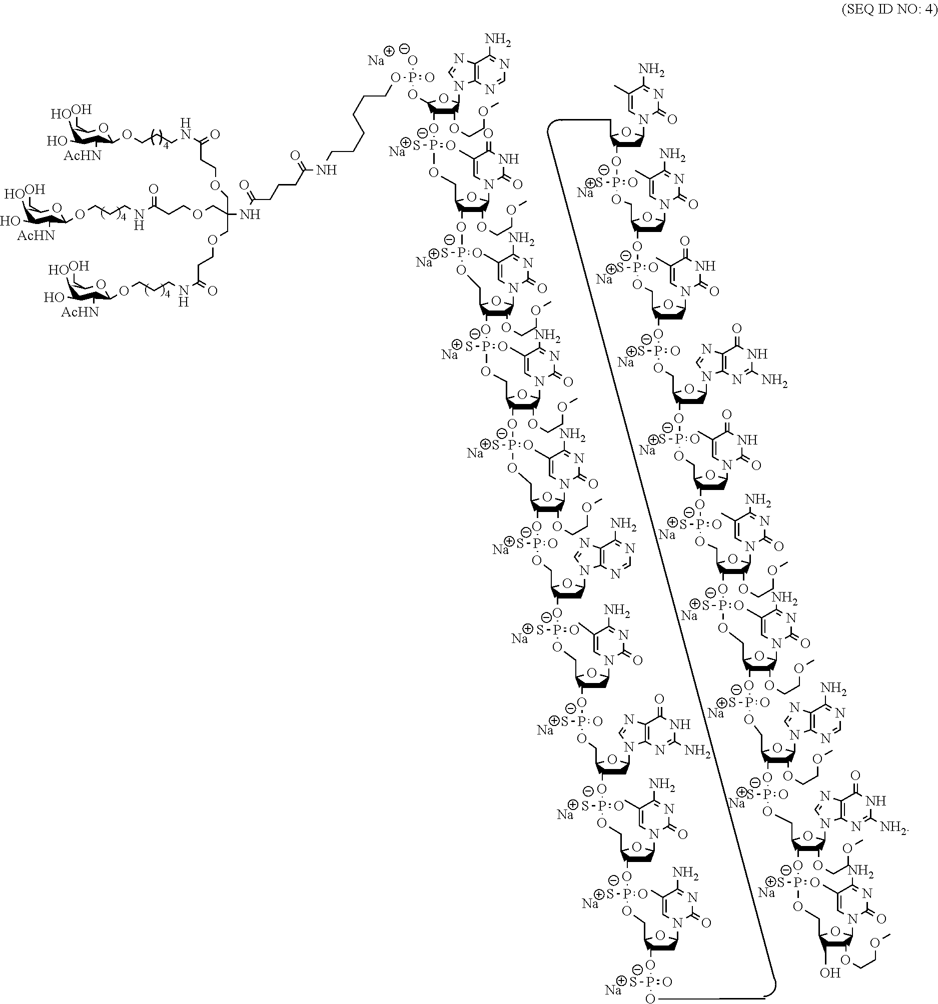

Embodiment 170. A method of ameliorating IgA Nephropathy in a human subject in need thereof, the method comprising subcutaneously administering to the human subject 70 mg, or about 70 mg of a compound, wherein the compound has the following chemical notation (5′ to 3′): GalNAc₃-7_{a-o′} Aₑₛ Tₑₛ^{m}Cₑₛ^{m}Cₑₛ^{m}CₑₛA_{ds}^{m}C_{ds}G_{ds}^{m}C_{ds}^{m}C_{ds}^{m}C_{ds}^{m}C_{ds} T_{ds}G_{ds}T_{ds}^{m}CₑₛCₑₛAₑₛGₑₛ^{m}Cₑ (SEQ ID NO: 4); wherein,
A=an adenine nucleobase,
^{m}C=a 5-methylcytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and

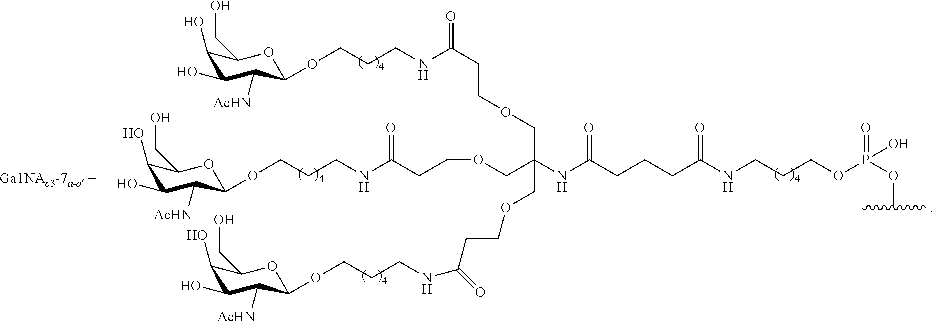

Embodiment 171. A method of ameliorating Geographic Atrophy in a human subject in need thereof, the method comprising subcutaneously administering to the human subject 40 mg, about 40 mg, 70 mg, or about 70 mg of a compound according to the following chemical structure:

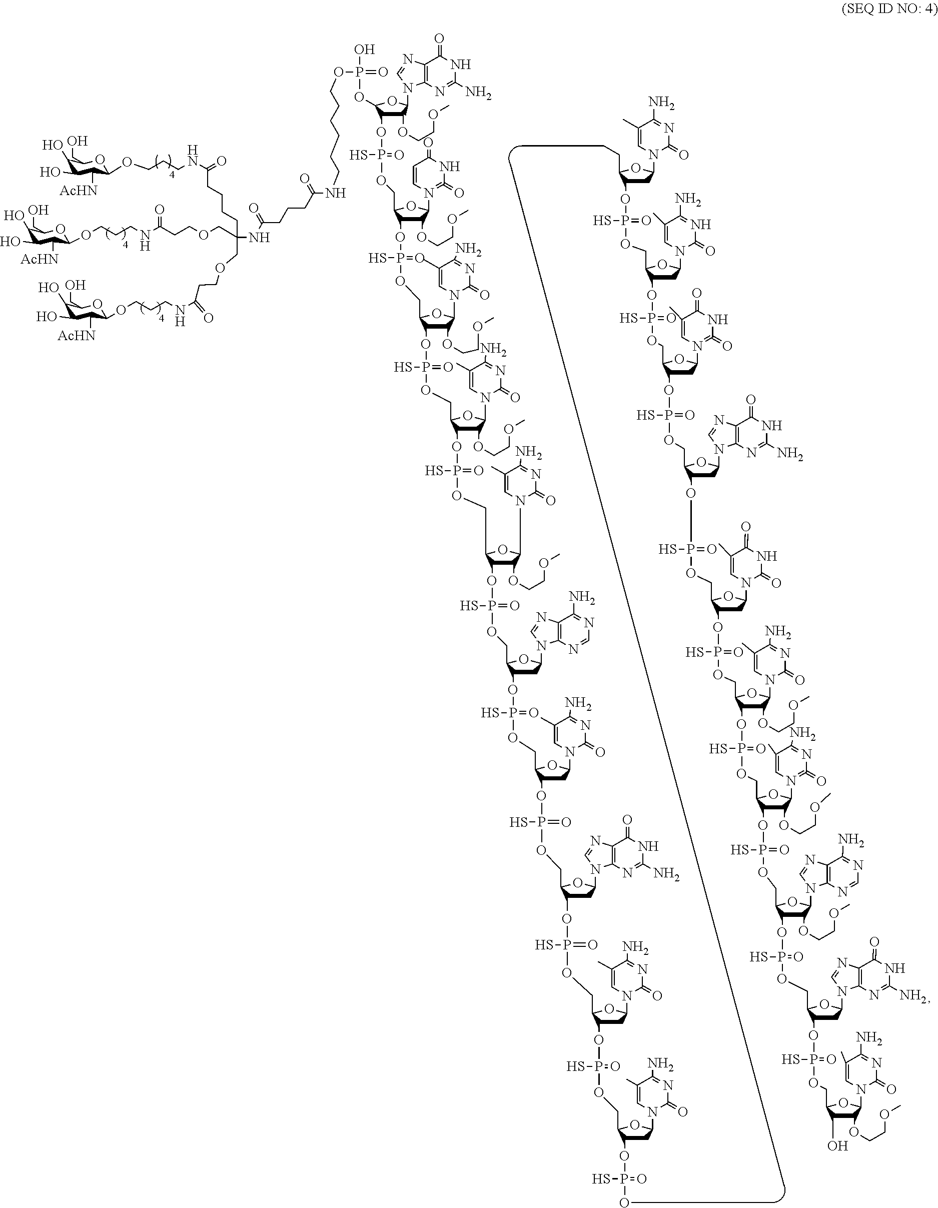

or a pharmaceutically acceptable salt thereof.

Embodiment 172. The method of embodiment 171, wherein the compound is the sodium salt or the potassium salt.

Embodiment 173. A method of ameliorating Geographic Atrophy in a human subject in need thereof, the method comprising subcutaneously administering to the human subject 40 mg, about 40 mg, 70 mg, or about 70 mg of a compound according to the following chemical structure:

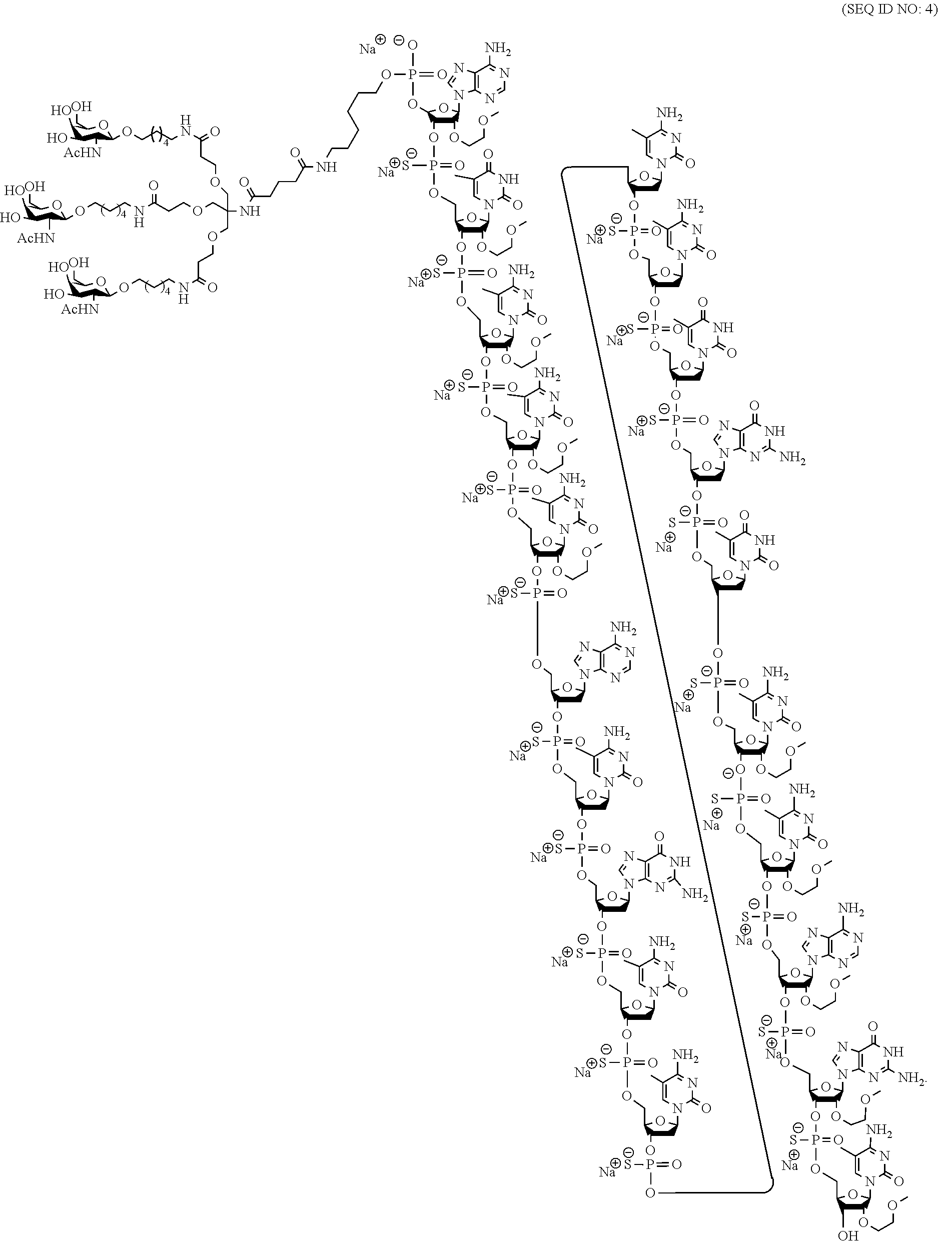

Embodiment 174. A method of ameliorating Geographic Atrophy in a human subject in need thereof, the method comprising subcutaneously administering to the human subject 40 mg, about 40 mg, 70 mg, or about 70 mg of a compound, wherein the compound has the following chemical notation (5′ to 3′): GalNAc₃-7_{a-o′}
Aₑₛ Tₑₛ ^{m}Cₑₛ^{m}Cₑₛ ^{m}CₑₛA_{ds}^{m}C_{ds}G_{ds}^{m}C_{ds}^{m}C_{ds}^{m}C_{ds}^{m}C_{ds}T_{ds}G_{ds} T_{ds}^{m}Cₑₛ^{m}CₑₛAₑₛGₑₛ^{m}Cₑ (SEQ ID NO: 4); wherein,
   A=an adenine nucleobase,
   ^{m}C=a 5-methylcytosine nucleobase,
   G=a guanine nucleobase,
   T=a thymine nucleobase,
   e=a 2′-MOE sugar moiety,
   d=a 2′-β-D-deoxyribosyl sugar moiety,
   s=a phosphorothioate internucleoside linkage, and

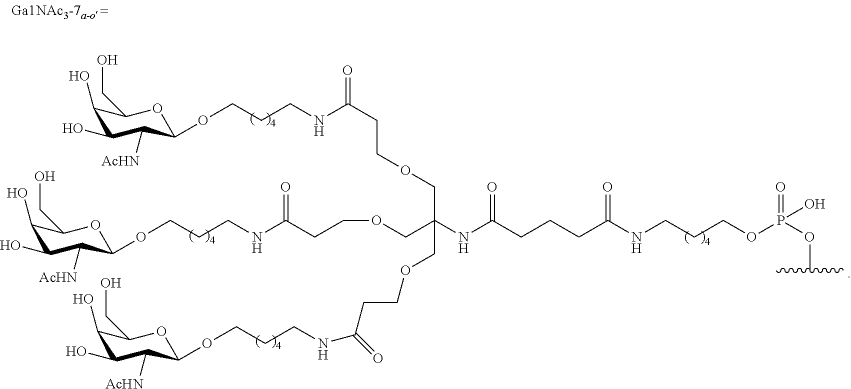

Embodiment 175. The method of any of embodiments 163-174, comprising administering the compound once every 4 weeks.

Embodiment 176. The method of any of embodiments 163-174, comprising administering the compound once every 8 weeks.

Embodiment 177. The method of any of embodiments 163-174, comprising administering the compound once every 12 weeks.

Embodiment 178. The method of any of embodiments 163-174, comprising administering the compound once every 16 weeks.

Embodiment 179. The method of any of embodiments 163-174, comprising administering the compound once every 24 weeks.

Embodiment 180. The method of any of embodiments 163-174, comprising administering the compound once every 6 months.

Embodiment 181. The method of any of embodiments 163-174, comprising administering the compound about once every 4 weeks.

Embodiment 182. The method of any of embodiments 163-174, comprising administering the compound about once every 8 weeks.

Embodiment 183. The method of any of embodiments 163-174, comprising administering the compound about once every 12 weeks.

Embodiment 184. The method of any of embodiments 163-174, comprising administering the compound about once every 16 weeks.

Embodiment 185. The method of any of embodiments 163-174, comprising administering the compound about once every 24 weeks.

Embodiment 186. The method of any of embodiments 163-174, comprising administering the compound about once every 6 months.

Embodiment 187. The method of any of embodiments 163-174, comprising administering to the human subject a dose of 40 mg of the compound once every four weeks.

Embodiment 188. The method of any of embodiments 163-174, comprising administering to the human subject a dose of 70 mg of the compound once every four weeks.

Embodiment 189. The method of any of embodiments 163-174, comprising administering to the human subject a dose of 100 mg of the compound once every four weeks.

Embodiment 190. The method of any of embodiments 163-174, wherein the compound is administered by injection.

Embodiment 191. The method of any of embodiments 163-190, wherein the compound is formulated as a pharmaceutical composition comprising the compound and a pharmaceutically acceptable diluent.

Embodiment 192. The method of embodiment 191, wherein the pharmaceutically acceptable diluent is sterile water, sterile saline, or sterile phosphate buffered saline.

Embodiment 193. The method of any of embodiment 163-192, wherein administering the compound reduces the level of ocular C3 deposits, or reduces accumulation of ocular C3 deposits, or slows the progression of Geographic Atrophy in the subject.

Embodiment 194. The method of any of embodiments 163-192, wherein administering the compound reduces the level of plasma CFB protein, reduces serum AP activity, reduces the level of serum functional CFB, reduces the level of urinary Factor B split product (Ba), or reduces the level of proteinuria in the subject.

Embodiment 195. The method of embodiment 194, wherein the urine protein/creatine ratio is reduced following administration of the compound.

Embodiment 196. The method of embodiment 194, wherein the level of urine protein is reduced following administration of the compound.

Embodiment 197. The method of any of embodiments 163-192, wherein administration of the compound slows the progression of Geographic Atrophy in the subject.

Embodiment 198. The method of embodiment 163-193, wherein administration of the compound slows the expansion of Geographic Atrophy Area.

Embodiment 199. The method of any of embodiments 163-193, wherein administration of the compound slows the expansion of Geographic Atrophy into the fovea region.

Embodiment 200. The method of embodiment 163-193, wherein administration of the compound stops expansion of Geographic Atrophy.

Embodiment 201. The method of any of embodiments 1-200, comprising detecting an amount of CFB protein or CFB RNA in a biological sample from the human subject.

Embodiment 202. The method of embodiment 201, wherein the biological sample is blood.

Embodiment 203. The method of embodiment 102, wherein the biological sample is serum or plasma.

Embodiment 204. The method of embodiment 201, wherein the biological sample is urine.

Embodiment 205. The method of any of embodiments 201-204, wherein the detecting occurs before administering the compound.

Embodiment 206. The method of any of embodiments 201-204-178, wherein the detecting occurs after administering the compound.

Embodiment 207. The method of any of embodiments 201-204, wherein the detecting occurs before and after administering the compound.

Embodiment 208. The method of any of embodiments 201-207, comprising adjusting the initial loading dose, the loading dose, maintenance dose, or therapeutically effective amount of compound administered after detecting the amount of CFB RNA, CFB protein, or combination thereof.

Embodiment 209. The method of any of embodiments 1-208, comprising analyzing the level of plasma CFB protein, the level of serum AP activity, the level of serum functional CFB, the level of urinary Factor B split product (Ba), or the level of urinary protein in the subject.

Embodiment 210. The method of embodiment 209, wherein the urine protein/creatine ratio in a urine sample from the subject is analyzed after administering the compound.

Embodiment 211. The method of embodiment 209, wherein the level of urine protein in a urine sample from the subject is analyzed after administering the compound.

Embodiment 212. The method of embodiment 210 or embodiment 211, wherein the analysis occurs before administering the compound, or after administering the compound, or a combination thereof.

Embodiment 213. The method of embodiment 212, comprising determining or adjusting the therapeutically effective amount of the compound after performing the analysis.

Embodiment 214. The method of embodiment 212 or embodiment 213, comprising performing the analysis after administering the compound, and adjusting the frequency of administering the compound after performing the analysis.

### I. Complement Factor B (CFB)

In certain embodiments, described herein are methods of reducing CFB RNA and/or CFB protein in a cell or a biological fluid of a subject. CFB RNA is encoded by the human CFB gene, located on human 6 position 31913721-31919861. CFB protein is highly expressed in liver relative to other cell types. A representative nucleobase sequence for a human CFB RNA is provided at GENBANK Accession No. NM_001710.5 (incorporated herein as SEQ ID NO: 1). A representative nucleobase sequence for a human CFB gene is provided at GENBANK Accession No. NT 007592.15 truncated from nucleotides 31852000 to 31861000 (incorporated herein as SEQ ID NO: 2). A representative nucleobase sequence for a human CFB gene is provided at GENBANK Accession No. NC_000006.12 truncated from nucleotides 31943001 to 31955000 (SEQ ID NO: 5). A representative nucleobase sequence for a human CFB gene is provided at ENSEMBL Gene ID ENSG00000243649.10, Ensembl Release 108 (October 2022) (SEQ ID NO: 6).

### II. Compound 696844

In certain embodiments, described herein are methods of administering Compound 696844 (FB-LRₓ), to a subject in need thereof. In certain embodiments, Compound 696844 is characterized as a 5-10-5 MOE gapmer covalently bound at the 5′ end to triantennary N-acetyl galactosamine (GalNAc₃). Compound 696844 has a sequence of (from 5′ to 3′) ATCCCACGCCCCTGTCCAGC (SEQ ID NO: 3), wherein each of nucleosides 1-5 and 16-20 are 2′-MOE nucleosides and each of nucleosides 6-15 are 2′-β-D-deoxynucleosides, wherein all of the internucleoside linkages are phosphorothioate internucleoside linkages, and wherein each cytosine is a 5-methylcytosine.

In certain embodiments, Compound 696844 is characterized by the following chemical notation (5′ to 3′): GalNAc₃-7_{a-o′}
Aₑₛ Tₑₛ^{m}Cₑₛ^{m}Cₑₛ^{m}CₑₛA_{ds}^{m}C_{ds}G_{ds}^{m}C_{ds}^{m}C_{ds}^{m}C_{ds}^{m}C_{ds} T_{ds}G_{ds} T_{ds}^{m}Cₑₛ^{m}CₑₛAₑₛGₑₛ^{m}Cₑ (SEQ ID NO: 4); wherein,
   A=an adenine nucleobase,
   ^{m}C=a 5-methylcytosine nucleobase,
   G=a guanine nucleobase,
   T=a thymine nucleobase,
   e=a 2′-MOE sugar moiety,
   d=a 2′-β-D-deoxyribosyl sugar moiety,
   s=a phosphorothioate internucleoside linkage, and

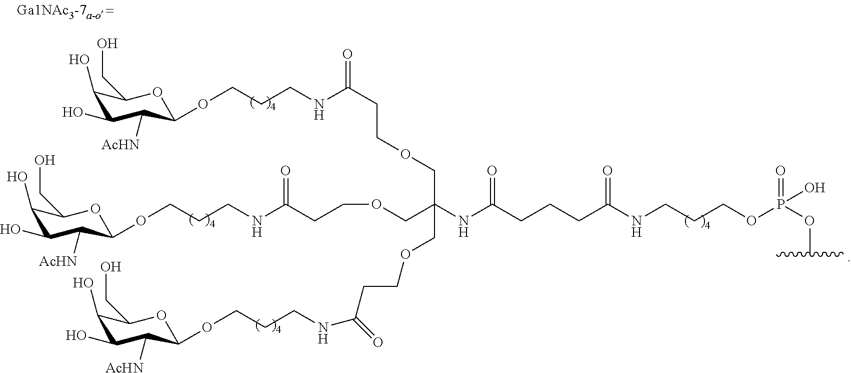

In certain embodiments, Compound 696844 is characterized by the following chemical structure:

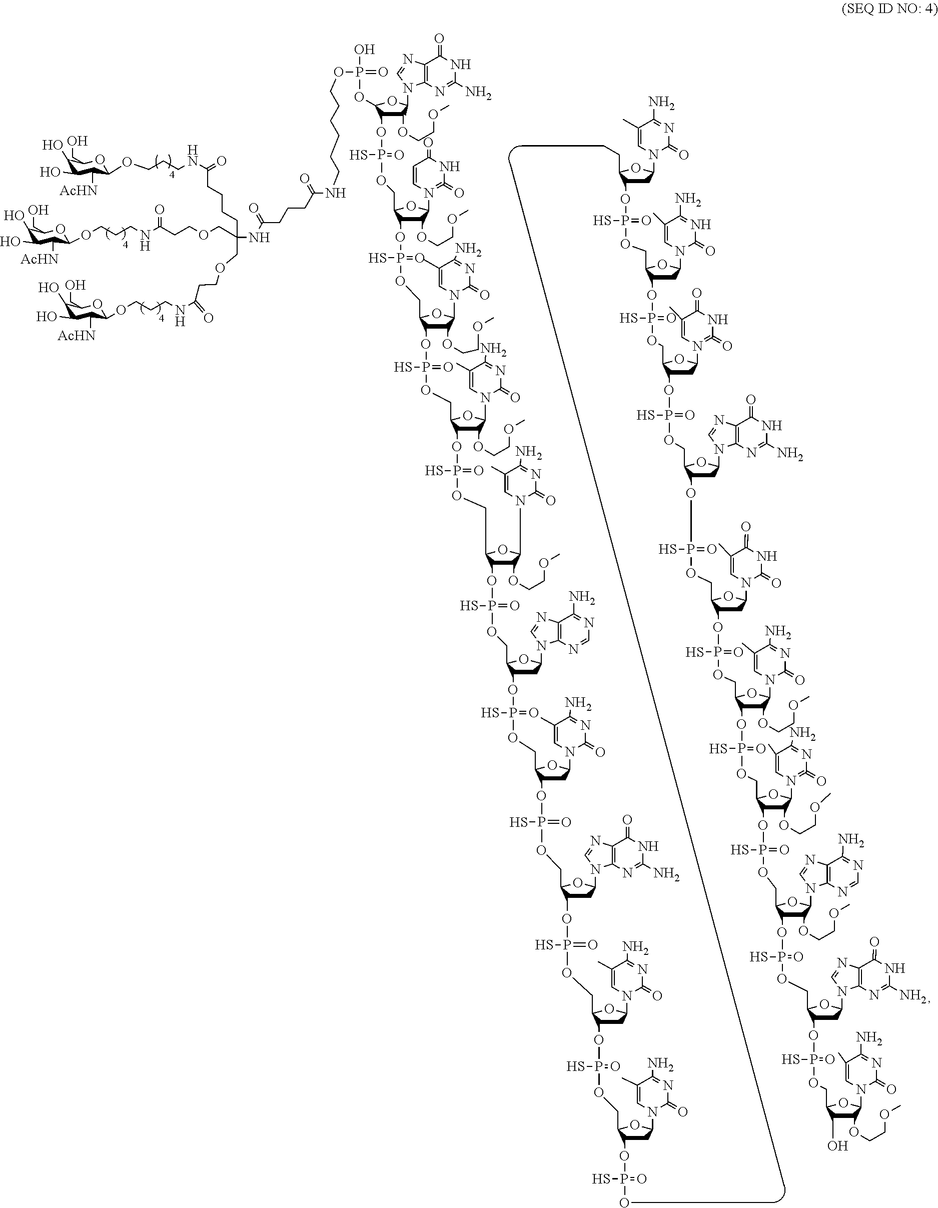

### Structure 1. Compound 696844

In certain embodiments, the sodium salt of Compound 696844 is represented by the following chemical structure:

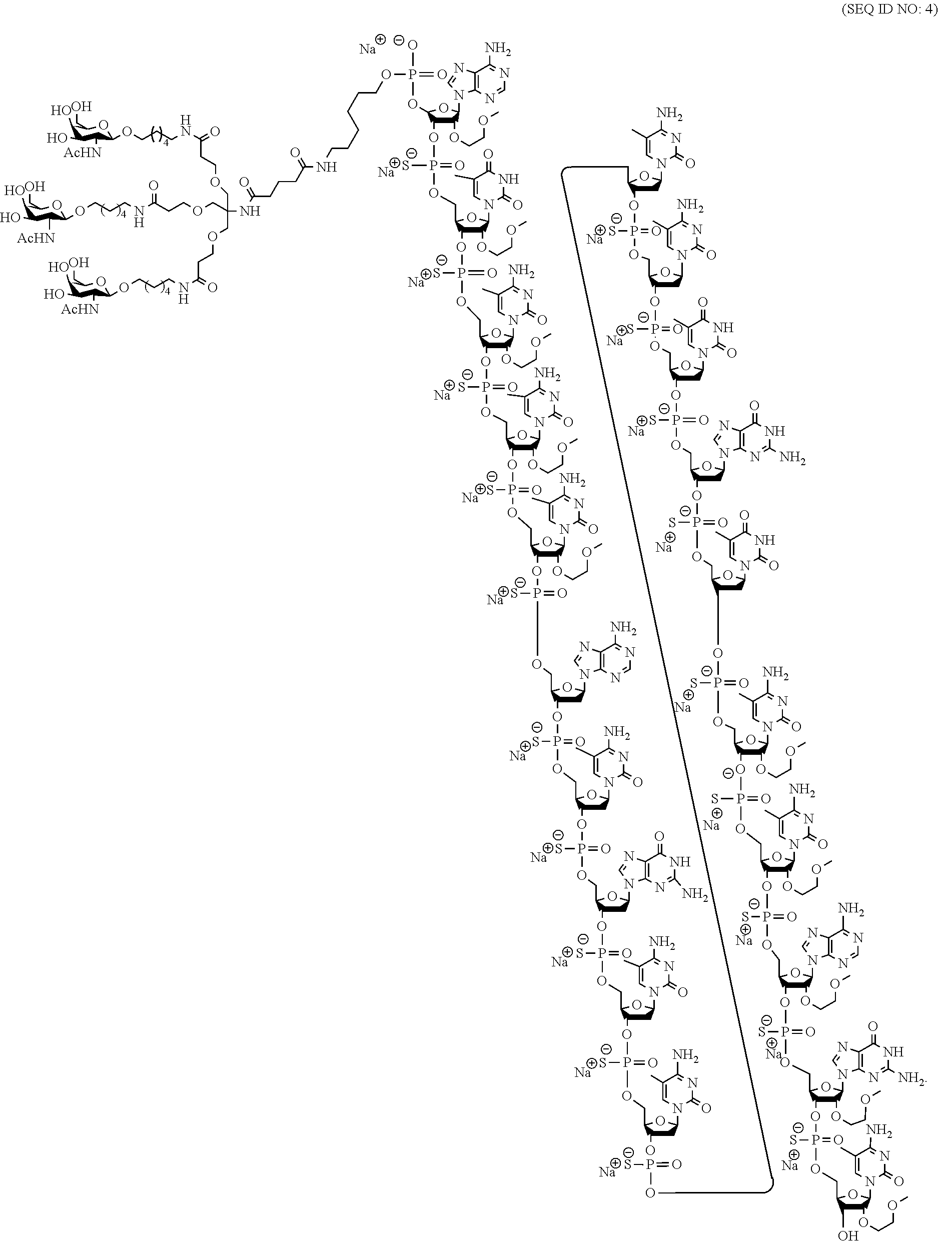

Structure 2. Sodium salt of Compound 696844

In certain embodiments, Compound 696844 is characterized as an icosasodium salt of a 20-base residue (20-mer) oligonucleotide conjugated at its 5′ end via an aminohexylphosphate linker to a GalNAc THA cluster. Each of the nineteen internucleotide linkages is a 3′-0 to 5′-O phosphorothioate diester. Ten (10) of the 20 sugar residues are 2-deoxy-D-ribose and the remainder are 2′-O-(2-methoxyethyl)-D-ribose (MOE). The residues are arranged so that there are 5 MOE nucleosides at the 5′ and 3′ ends of the molecule flanking a gap of ten 2′-deoxynucleosides. The cytosine bases are methylated at the 5-position. The sequence can be written in shorthand as follows:

5′-THA-AHoA^{Me}U^{Me}C^{Me}C^{Me}CA^{Me}CG^{Me}C^{Me}C^{Me}c^{Me}
CTGT^{Me}C^{Me}CAG^{Me}C-3′

The underlined residues are 2′-MOE nucleosides. AH designates the position of the aminohexyl linker; o designates a phosphodiester linkage; THA is 5-N-[tris({6-[(2-acetamido-2-deoxy-β-D-galactopyranosyl)oxy]-hexylamino}-3-oxopropoxymethyl)methyl]amino-5-oxopentanoyl, 2′-O-(2-methoxyethyl)-methyluridine (2′-MOE ^{Me}U) nucleosides are sometimes referred to as 2′-O-(2-methoxyethyl)-ribothymidine (2′-MOE T).

In certain embodiments, Compound 696844 is characterized by the following nomenclature, showing each 3′-O-linked phosphorothioate diester internucleotide linkage as follows: 5′-O-(6-{5-N-[tris({6-[(2-acetamido-2-deoxy-β-D-galactopyranosyl)oxy]-hexylamino}-3-oxopropoxymethyl)methyl]amino-5-oxopentanoyl}aminohexyl-1-phosphatyl)-2□-O-(2-methoxyethyl)-P-thioadenylyl-(3□-O□5□-O)-2□-□-(2-methoxyethyl)-5-methyl-P-thiouridylyl-(3□-O□5□-O)-2□-□-(2-methoxyethyl)-5-methyl-P-thiocytidylyl-(3□-O□5□-O)-2□-□-(2-methoxyethyl)-5-methyl-P-thiocytidylyl-(3□-O□5□-O)-2□-□-(2-methoxyethyl)-5-methyl-P-thiocytidylyl-(3□-O□5□-O)-2□-deoxy-P-thioadenylyl-(3□-O□5□-O)-2□-deoxy-5-methyl-P-thiocytidylyl-(3□-O□5□-O)-2□-deoxy-P-thioguanylyl-(3□-O□5□-O)-2□-deoxy-5-methyl-P-thiocytidylyl-(3□-O□5□-O)-2□-deoxy-5-methyl-P-thiocytidylyl-(3□-O□5□-O)-2□-deoxy-5-methyl-P-thiocytidylyl-(3□-O□5□-O)-2□-deoxy-5-methyl-P-thiocytidylyl-(3□-O□5□-O)—P-thiothymidylyl-(3□-O□5□-O)-2□-deoxy-P-thioguanylyl-(3□-O□5□-O)—P-thiothymidylyl-(3□-O□5□-O)-2□-□-(2-methoxyethyl)-5-methyl-P-thiocytidylyl-(3□-O□5□-O)-2□-□-(2-methoxyethyl)-5-methyl-P-thiocytidylyl-(3□-O□5□-O)-2□-O-(2-methoxyethyl)-P-thioadenylyl-(3□-O□5□-O)-2□-□-(2-methoxyethyl)-P-thioguanylyl-(3□-O□5□-O)-2□-□-(2-methoxyethyl)-5-methylcytidine, 20 sodium salt.

In certain embodiments, the Compound 696844 pharmaceutical composition is a sterile, parenteral solution of 100 mg/mL Compound 696844 in phosphate buffered saline. The formulation contains Compound 696844 at 100 mg/mL adjusted to approximately pH 7.4 with acid or base during compounding. The solution is clear and colorless to light yellow in color. In certain embodiments, the pharmaceutical composition is packaged as a 0.8 mL deliverable volume in an ISO 2R Type I, clear glass vial that is stoppered with a butyl rubber closure and sealed with an aluminum overseal. In certain embodiments, approximately 1.0 mL is filled into each vial to ensure the labeled 0.8 mL volume is available for dosing. In certain embodiments, the pharmaceutical composition is for single use and contains no preservatives. In certain embodiments, the pharmaceutical composition is stored securely at 2-8° C., protected from light.

Compound 696844, and pharmaceutical compositions comprising Compound 696844, are described in WO 2015/168635.

### III. Certain Pharmaceutical Compositions

In certain embodiments, described herein are methods of administering to a subject a pharmaceutical composition comprising the Compound 696844. In certain embodiments, the pharmaceutical composition comprises a pharmaceutically acceptable diluent or carrier. In certain embodiments, the pharmaceutical composition comprises or consists essentially of a sterile saline solution and the Compound 696844. In certain embodiments, the sterile saline is pharmaceutical grade saline. In certain embodiments, the pharmaceutical composition comprises or consists essentially of sterile water and the Compound 696844. In certain embodiments, the sterile water is pharmaceutical grade water. In certain embodiments, the pharmaceutical composition comprises or consists essentially of phosphate buffered saline (PBS) and the Compound 696844. In certain embodiments, the PBS is pharmaceutical grade. In certain embodiments, the pharmaceutical composition comprises a certain concentration of Compound 696844 in PBS, and is diluted in a diluent to achieve the intended clinical dose. In certain embodiments, the diluent is sterile saline solution, sterile water, or PBS. In certain embodiments, the pharmaceutical composition comprises a certain concentration of Compound 696844 in PBS, and is diluted in PBS to achieve the intended clinical dose. In certain embodiments, Compound 696844 is formulated in phosphate buffered saline (PBS) at 100 mg/mL. In certain embodiments, Compound 696844 is formulated in PBS at 100 mg/mL and is in a stoppered and sealed glass vial. In certain embodiments, Compound 696844 is formulated in PBS at 100 mg/mL and is provided for clinical use as a 0.8 mL deliverable volume. In certain embodiments, Compound 696844 is formulated in PBS at 100 mg/mL and is provided for clinical use as a 0.8 mL deliverable volume in a stoppered and sealed glass vial. In certain embodiments, Compound 696844 is formulated in PBS at 100 mg/mL and is diluted in PBS to achieve the intended clinical dose. The term “deliverable volume” may include an additional amount of the formulation to ensure the deliverable volume is available for dosing. In certain embodiments, the glass vial contains 1.0 mL of the formulation, for a 0.8 mL deliverable volume.

In certain embodiments, pharmaceutical compositions comprise one or more excipients and the Compound 696844. In certain embodiments, excipients are selected from water, salt solutions, alcohol, polyethylene glycols, gelatin, lactose, amylase, magnesium stearate, talc, silicic acid, viscous paraffin, hydroxymethylcellulose, and polyvinylpyrrolidone.

In certain embodiments, pharmaceutical compositions comprising the Compound 696844 encompass any pharmaceutically acceptable salt of the Compound 696844, esters of the Compound 696844, or salts of such esters. In certain embodiments, the pharmaceutically acceptable salt comprises sodium, potassium, calcium, or magnesium. In certain embodiments, the pharmaceutically acceptable salt comprises sodium or magnesium. In certain embodiments, the pharmaceutical composition comprises one or more of sodium, potassium, calcium, or magnesium. In certain embodiments, pharmaceutical compositions comprise a pharmaceutically acceptable salt of the compound represented by Structure 1 comprising one or more cations selected from sodium, potassium, calcium, and magnesium. In certain embodiments, pharmaceutical compositions comprising the Compound 696844 are capable of providing (directly or indirectly) the biologically active metabolite or residue thereof upon administration to a human subject. Accordingly, for example, the disclosure is also drawn to pharmaceutically acceptable salts of the Compound 696844, prodrugs of the Compound 696844, pharmaceutically acceptable salts of such prodrugs, and other bioequivalents.

In certain embodiments, pharmaceutical compositions comprise one or more lipid moieties and the Compound 696844. In certain embodiments, lipid moieties are used to increase distribution of Compound 696844 to a particular cell or tissue. In certain such methods, the Compound 696844 is introduced into preformed liposomes or lipoplexes made of mixtures of cationic lipids and neutral lipids. In certain methods, DNA complexes with mono- or poly-cationic lipids are formed without the presence of a neutral lipid.

In certain embodiments, pharmaceutical compositions disclosed herein comprise a delivery system. Examples of delivery systems include, but are not limited to, liposomes and emulsions. Certain delivery systems are useful for preparing pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver compounds described herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV)). In certain of such embodiments, a pharmaceutical composition comprises a carrier and is formulated in aqueous solution, such as water, or physiologically compatible buffers such as phosphate buffered saline (PBS), Hanks's solution, Ringer's solution, physiological saline buffer, or artificial CSF. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are presented in unit dosage form in pre-filled syringes. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, the Compound 696844 acts as an acid. Although Compound 696844 may be drawn or described in protonated (free acid) form, or ionized and in association with a cation (salt) form, aqueous solutions of Compound 696844 exist in equilibrium among such forms. For example, a phosphate linkage of Compound 696844 in aqueous solution exists in equilibrium among free acid, anion, and salt forms. Unless otherwise indicated, the term, “Compound 696844,” is intended to include all such forms. Moreover, Compound 696844 has several such linkages, each of which is in equilibrium. Thus, Compound 696844 exists in solution in an ensemble of forms at multiple positions all at equilibrium. The term “Compound 696844” is intended to include all such forms. Drawn structures necessarily depict a single form. Nevertheless, unless otherwise indicated, such drawings are likewise intended to include corresponding forms. Herein, a structure depicting the free acid of Compound 696844 followed by the term “or a salt thereof” expressly includes all such forms that may be fully or partially protonated/de-protonated/in association with a cation. In certain instances, one or more specific cation is identified.

In certain embodiments, Compound 696844 is in aqueous solution with sodium. In certain embodiments, Compound 696844 is in aqueous solution with potassium. In certain embodiments, Compound 696844 is in PBS. In certain embodiments, Compound 696844 is in water. In certain such embodiments, the pH of the solution is adjusted with NaOH and/or HCl to achieve a desired pH.

Herein, certain specific doses are described. For clarity, a dose of Compound 696844 in milligrams indicates the mass of the free acid form of Compound 696844. As described above, in aqueous solution, the free acid is in equilibrium with anionic and salt forms. However, for the purpose of calculating dose, it is assumed that Compound 696844 exists as a solvent-free, sodium-acetate free, anhydrous, free acid. For example, where Compound 696844 is in solution comprising sodium (e.g., saline), Compound 696844 may be partially or fully de-protonated and in association with Na+ ions. However, the mass of the protons is nevertheless counted toward the weight of the dose, and the mass of the Na+ ions are not counted toward the weight of the dose. Thus, for example, a dose of 40 mg of Compound 696844 equals the number of fully protonated molecules that weighs 40 mg. This would be equivalent to 40.93 mg of solvent-free, sodium-acetate free, anhydrous sodiated Compound 696844. Similarly, a dose of 70 mg of Compound 696844 equals the number of fully protonated molecules that weighs 70 mg. This would be equivalent to 73.37 mg of solvent-free, sodium-acetate free, anhydrous sodiated Compound 696844.

### IV. Certain Dosage Amounts

In certain embodiments, described herein are methods of administering to a subject a therapeutically effective amount of the Compound 696844. In certain embodiments, the therapeutically effective amount is 10 mg. In certain embodiments, the therapeutically effective amount is 20 mg. In certain embodiments, the therapeutically effective amount is 40 mg. In certain embodiments, the therapeutically effective amount is 70 mg. In certain embodiments, the therapeutically effective amount is 100 mg.

In certain embodiments, the therapeutically effective amount is any of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, and 350 mg.

In certain embodiments, the therapeutically effective amount is any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, and about 350 mg.

In certain embodiments, the therapeutically effective amount is any of 10 mg, 10.1 mg, 10.2 mg, 10.3 mg, 10.4 mg, 10.5 mg, 10.6 mg, 10.7 mg, 10.8 mg, 10.9 mg, 11 mg, 11.1 mg, 11.2 mg, 11.3 mg, 11.4 mg, 11.5 mg, 11.6 mg, 11.7 mg, 11.8 mg, 11.9 mg, 12 mg, 12.1 mg, 12.2 mg, 12.3 mg, 12.4 mg, 12.5 mg, 12.6 mg, 12.7 mg, 12.8 mg, 12.9 mg, 13 mg, 13.1 mg, 13.2 mg, 13.3 mg, 13.4 mg, 13.5 mg, 13.6 mg, 13.7 mg, 13.8 mg, 13.9 mg, 14 mg, 14.1 mg, 14.2 mg, 14.3 mg, 14.4 mg, 14.5 mg, 14.6 mg, 14.7 mg, 14.8 mg, 14.9 mg, 15 mg, 15.1 mg, 15.2 mg, 15.3 mg, 15.4 mg, 15.5 mg, 15.6 mg, 15.7 mg, 15.8 mg, 15.9 mg, 16 mg, 16.1 mg, 16.2 mg, 16.3 mg, 16.4 mg, 16.5 mg, 16.6 mg, 16.7 mg, 16.8 mg, 16.9 mg, 17 mg, 17.1 mg, 17.2 mg, 17.3 mg, 17.4 mg, 17.5 mg, 17.6 mg, 17.7 mg, 17.8 mg, 17.9 mg, 18 mg, 18.1 mg, 18.2 mg, 18.3 mg, 18.4 mg, 18.5 mg, 18.6 mg, 18.7 mg, 18.8 mg, 18.9 mg, 19 mg, 19.1 mg, 19.2 mg, 19.3 mg, 19.4 mg, 19.5 mg, 19.6 mg, 19.7 mg, 19.8 mg, 19.9 mg, 20 mg, 20.1 mg, 20.2 mg, 20.3 mg, 20.4 mg, 20.5 mg, 20.6 mg, 20.7 mg, 20.8 mg, 20.9 mg, 21 mg, 21.1 mg, 21.2 mg, 21.3 mg, 21.4 mg, 21.5 mg, 21.6 mg, 21.7 mg, 21.8 mg, 21.9 mg, 22 mg, 22.1 mg, 22.2 mg, 22.3 mg, 22.4 mg, 22.5 mg, 22.6 mg, 22.7 mg, 22.8 mg, 22.9 mg, 23 mg, 23.1 mg, 23.2 mg, 23.3 mg, 23.4 mg, 23.5 mg, 23.6 mg, 23.7 mg, 23.8 mg, 23.9 mg, 24 mg, 24.1 mg, 24.2 mg, 24.3 mg, 24.4 mg, 24.5 mg, 24.6 mg, 24.7 mg, 24.8 mg, 24.9 mg, 25 mg, 25.1 mg, 25.2 mg, 25.3 mg, 25.4 mg, 25.5 mg, 25.6 mg, 25.7 mg, 25.8 mg, 25.9 mg, 26 mg, 26.1 mg, 26.2 mg, 26.3 mg, 26.4 mg, 26.5 mg, 26.6 mg, 26.7 mg, 26.8 mg, 26.9 mg, 27 mg, 27.1 mg, 27.2 mg, 27.3 mg, 27.4 mg, 27.5 mg, 27.6 mg, 27.7 mg, 27.8 mg, 27.9 mg, 28 mg, 28.1 mg, 28.2 mg, 28.3 mg, 28.4 mg, 28.5 mg, 28.6 mg, 28.7 mg, 28.8 mg, 28.9 mg, 29 mg, 29.1 mg, 29.2 mg, 29.3 mg, 29.4 mg, 29.5 mg, 29.6 mg, 29.7 mg, 29.8 mg, 29.9 mg, 30 mg, 30.1 mg, 30.2 mg, 30.3 mg, 30.4 mg, 30.5 mg, 30.6 mg, 30.7 mg, 30.8 mg, 30.9 mg, 31 mg, 31.1 mg, 31.2 mg, 31.3 mg, 31.4 mg, 31.5 mg, 31.6 mg, 31.7 mg, 31.8 mg, 31.9 mg, 32 mg, 32.1 mg, 32.2 mg, 32.3 mg, 32.4 mg, 32.5 mg, 32.6 mg, 32.7 mg, 32.8 mg, 32.9 mg, 33 mg, 33.1 mg, 33.2 mg, 33.3 mg, 33.4 mg, 33.5 mg, 33.6 mg, 33.7 mg, 33.8 mg, 33.9 mg, 34 mg, 34.1 mg, 34.2 mg, 34.3 mg, 34.4 mg, 34.5 mg, 34.6 mg, 34.7 mg, 34.8 mg, 34.9 mg, 35 mg, 35.1 mg, 35.2 mg, 35.3 mg, 35.4 mg, 35.5 mg, 35.6 mg, 35.7 mg, 35.8 mg, 35.9 mg, 36 mg, 36.1 mg, 36.2 mg, 36.3 mg, 36.4 mg, 36.5 mg, 36.6 mg, 36.7 mg, 36.8 mg, 36.9 mg, 37 mg, 37.1 mg, 37.2 mg, 37.3 mg, 37.4 mg, 37.5 mg, 37.6 mg, 37.7 mg, 37.8 mg, 37.9 mg, 38 mg, 38.1 mg, 38.2 mg, 38.3 mg, 38.4 mg, 38.5 mg, 38.6 mg, 38.7 mg, 38.8 mg, 38.9 mg, 39 mg, 39.1 mg, 39.2 mg, 39.3 mg, 39.4 mg, 39.5 mg, 39.6 mg, 39.7 mg, 39.8 mg, 39.9 mg, 40 mg, 40.1 mg, 40.2 mg, 40.3 mg, 40.4 mg, 40.5 mg, 40.6 mg, 40.7 mg, 40.8 mg, 40.9 mg, 41 mg, 41.1 mg, 41.2 mg, 41.3 mg, 41.4 mg, 41.5 mg, 41.6 mg, 41.7 mg, 41.8 mg, 41.9 mg, 42 mg, 42.1 mg, 42.2 mg, 42.3 mg, 42.4 mg, 42.5 mg, 42.6 mg, 42.7 mg, 42.8 mg, 42.9 mg, 43 mg, 43.1 mg, 43.2 mg, 43.3 mg, 43.4 mg, 43.5 mg, 43.6 mg, 43.7 mg, 43.8 mg, 43.9 mg, 44 mg, 44.1 mg, 44.2 mg, 44.3 mg, 44.4 mg, 44.5 mg, 44.6 mg, 44.7 mg, 44.8 mg, 44.9 mg, 45 mg, 45.1 mg, 45.2 mg, 45.3 mg, 45.4 mg, 45.5 mg, 45.6 mg, 45.7 mg, 45.8 mg, 45.9 mg, 46 mg, 46.1 mg, 46.2 mg, 46.3 mg, 46.4 mg, 46.5 mg, 46.6 mg, 46.7 mg, 46.8 mg, 46.9 mg, 47 mg, 47.1 mg, 47.2 mg, 47.3 mg, 47.4 mg, 47.5 mg, 47.6 mg, 47.7 mg, 47.8 mg, 47.9 mg, 48 mg, 48.1 mg, 48.2 mg, 48.3 mg, 48.4 mg, 48.5 mg, 48.6 mg, 48.7 mg, 48.8 mg, 48.9 mg, 49 mg, 49.1 mg, 49.2 mg, 49.3 mg, 49.4 mg, 49.5 mg, 49.6 mg, 49.7 mg, 49.8 mg, 49.9 mg, 50 mg, 50.1 mg, 50.2 mg, 50.3 mg, 50.4 mg, 50.5 mg, 50.6 mg, 50.7 mg, 50.8 mg, 50.9 mg, 51 mg, 51.1 mg, 51.2 mg, 51.3 mg, 51.4 mg, 51.5 mg, 51.6 mg, 51.7 mg, 51.8 mg, 51.9 mg, 52 mg, 52.1 mg, 52.2 mg, 52.3 mg, 52.4 mg, 52.5 mg, 52.6 mg, 52.7 mg, 52.8 mg, 52.9 mg, 53 mg, 53.1 mg, 53.2 mg, 53.3 mg, 53.4 mg, 53.5 mg, 53.6 mg, 53.7 mg, 53.8 mg, 53.9 mg, 54 mg, 54.1 mg, 54.2 mg, 54.3 mg, 54.4 mg, 54.5 mg, 54.6 mg, 54.7 mg, 54.8 mg, 54.9 mg, 55 mg, 55.1 mg, 55.2 mg, 55.3 mg, 55.4 mg, 55.5 mg, 55.6 mg, 55.7 mg, 55.8 mg, 55.9 mg, 56 mg, 56.1 mg, 56.2 mg, 56.3 mg, 56.4 mg, 56.5 mg, 56.6 mg, 56.7 mg, 56.8 mg, 56.9 mg, 57 mg, 57.1 mg, 57.2 mg, 57.3 mg, 57.4 mg, 57.5 mg, 57.6 mg, 57.7 mg, 57.8 mg, 57.9 mg, 58 mg, 58.1 mg, 58.2 mg, 58.3 mg, 58.4 mg, 58.5 mg, 58.6 mg, 58.7 mg, 58.8 mg, 58.9 mg, 59 mg, 59.1 mg, 59.2 mg, 59.3 mg, 59.4 mg, 59.5 mg, 59.6 mg, 59.7 mg, 59.8 mg, 59.9 mg, 60 mg, 60.1 mg, 60.2 mg, 60.3 mg, 60.4 mg, 60.5 mg, 60.6 mg, 60.7 mg, 60.8 mg, 60.9 mg, 61 mg, 61.1 mg, 61.2 mg, 61.3 mg, 61.4 mg, 61.5 mg, 61.6 mg, 61.7 mg, 61.8 mg, 61.9 mg, 62 mg, 62.1 mg, 62.2 mg, 62.3 mg, 62.4 mg, 62.5 mg, 62.6 mg, 62.7 mg, 62.8 mg, 62.9 mg, 63 mg, 63.1 mg, 63.2 mg, 63.3 mg, 63.4 mg, 63.5 mg, 63.6 mg, 63.7 mg, 63.8 mg, 63.9 mg, 64 mg, 64.1 mg, 64.2 mg, 64.3 mg, 64.4 mg, 64.5 mg, 64.6 mg, 64.7 mg, 64.8 mg, 64.9 mg, 65 mg, 65.1 mg, 65.2 mg, 65.3 mg, 65.4 mg, 65.5 mg, 65.6 mg, 65.7 mg, 65.8 mg, 65.9 mg, 66 mg, 66.1 mg, 66.2 mg, 66.3 mg, 66.4 mg, 66.5 mg, 66.6 mg, 66.7 mg, 66.8 mg, 66.9 mg, 67 mg, 67.1 mg, 67.2 mg, 67.3 mg, 67.4 mg, 67.5 mg, 67.6 mg, 67.7 mg, 67.8 mg, 67.9 mg, 68 mg, 68.1 mg, 68.2 mg, 68.3 mg, 68.4 mg, 68.5 mg, 68.6 mg, 68.7 mg, 68.8 mg, 68.9 mg, 69 mg, 69.1 mg, 69.2 mg, 69.3 mg, 69.4 mg, 69.5 mg, 69.6 mg, 69.7 mg, 69.8 mg, 69.9 mg, and 70 mg.

In certain embodiments, the therapeutically effective amount is any of about 10 mg, about 10.1 mg, about 10.2 mg, about 10.3 mg, about 10.4 mg, about 10.5 mg, about 10.6 mg, about 10.7 mg, about 10.8 mg, about 10.9 mg, about 11 mg, about 11.1 mg, about 11.2 mg, about 11.3 mg, about 11.4 mg, about 11.5 mg, about 11.6 mg, about 11.7 mg, about 11.8 mg, about 11.9 mg, about 12 mg, about 12.1 mg, about 12.2 mg, about 12.3 mg, about 12.4 mg, about 12.5 mg, about 12.6 mg, about 12.7 mg, about 12.8 mg, about 12.9 mg, about 13 mg, about 13.1 mg, about 13.2 mg, about 13.3 mg, about 13.4 mg, about 13.5 mg, about 13.6 mg, about 13.7 mg, about 13.8 mg, about 13.9 mg, about 14 mg, about 14.1 mg, about 14.2 mg, about 14.3 mg, about 14.4 mg, about 14.5 mg, about 14.6 mg, about 14.7 mg, about 14.8 mg, about 14.9 mg, about 15 mg, about 15.1 mg, about 15.2 mg, about 15.3 mg, about 15.4 mg, about 15.5 mg, about 15.6 mg, about 15.7 mg, about 15.8 mg, about 15.9 mg, about 16 mg, about 16.1 mg, about 16.2 mg, about 16.3 mg, about 16.4 mg, about 16.5 mg, about 16.6 mg, about 16.7 mg, about 16.8 mg, about 16.9 mg, about 17 mg, about 17.1 mg, about 17.2 mg, about 17.3 mg, about 17.4 mg, about 17.5 mg, about 17.6 mg, about 17.7 mg, about 17.8 mg, about 17.9 mg, about 18 mg, about 18.1 mg, about 18.2 mg, about 18.3 mg, about 18.4 mg, about 18.5 mg, about 18.6 mg, about 18.7 mg, about 18.8 mg, about 18.9 mg, about 19 mg, about 19.1 mg, about 19.2 mg, about 19.3 mg, about 19.4 mg, about 19.5 mg, about 19.6 mg, about 19.7 mg, about 19.8 mg, about 19.9 mg, about 20 mg, about 20.1 mg, about 20.2 mg, about 20.3 mg, about 20.4 mg, about 20.5 mg, about 20.6 mg, about 20.7 mg, about 20.8 mg, about 20.9 mg, about 21 mg, about 21.1 mg, about 21.2 mg, about 21.3 mg, about 21.4 mg, about 21.5 mg, about 21.6 mg, about 21.7 mg, about 21.8 mg, about 21.9 mg, about 22 mg, about 22.1 mg, about 22.2 mg, about 22.3 mg, about 22.4 mg, about 22.5 mg, about 22.6 mg, about 22.7 mg, about 22.8 mg, about 22.9 mg, about 23 mg, about 23.1 mg, about 23.2 mg, about 23.3 mg, about 23.4 mg, about 23.5 mg, about 23.6 mg, about 23.7 mg, about 23.8 mg, about 23.9 mg, about 24 mg, about 24.1 mg, about 24.2 mg, about 24.3 mg, about 24.4 mg, about 24.5 mg, about 24.6 mg, about 24.7 mg, about 24.8 mg, about 24.9 mg, about 25 mg, about 25.1 mg, about 25.2 mg, about 25.3 mg, about 25.4 mg, about 25.5 mg, about 25.6 mg, about 25.7 mg, about 25.8 mg, about 25.9 mg, about 26 mg, about 26.1 mg, about 26.2 mg, about 26.3 mg, about 26.4 mg, about 26.5 mg, about 26.6 mg, about 26.7 mg, about 26.8 mg, about 26.9 mg, about 27 mg, about 27.1 mg, about 27.2 mg, about 27.3 mg, about 27.4 mg, about 27.5 mg, about 27.6 mg, about 27.7 mg, about 27.8 mg, about 27.9 mg, about 28 mg, about 28.1 mg, about 28.2 mg, about 28.3 mg, about 28.4 mg, about 28.5 mg, about 28.6 mg, about 28.7 mg, about 28.8 mg, about 28.9 mg, about 29 mg, about 29.1 mg, about 29.2 mg, about 29.3 mg, about 29.4 mg, about 29.5 mg, about 29.6 mg, about 29.7 mg, about 29.8 mg, about 29.9 mg, about 30 mg, about 30.1 mg, about 30.2 mg, about 30.3 mg, about 30.4 mg, about 30.5 mg, about 30.6 mg, about 30.7 mg, about 30.8 mg, about 30.9 mg, about 31 mg, about 31.1 mg, about 31.2 mg, about 31.3 mg, about 31.4 mg, about 31.5 mg, about 31.6 mg, about 31.7 mg, about 31.8 mg, about 31.9 mg, about 32 mg, about 32.1 mg, about 32.2 mg, about 32.3 mg, about 32.4 mg, about 32.5 mg, about 32.6 mg, about 32.7 mg, about 32.8 mg, about 32.9 mg, about 33 mg, about 33.1 mg, about 33.2 mg, about 33.3 mg, about 33.4 mg, about 33.5 mg, about 33.6 mg, about 33.7 mg, about 33.8 mg, about 33.9 mg, about 34 mg, about 34.1 mg, about 34.2 mg, about 34.3 mg, about 34.4 mg, about 34.5 mg, about 34.6 mg, about 34.7 mg, about 34.8 mg, about 34.9 mg, about 35 mg, about 35.1 mg, about 35.2 mg, about 35.3 mg, about 35.4 mg, about 35.5 mg, about 35.6 mg, about 35.7 mg, about 35.8 mg, about 35.9 mg, about 36 mg, about 36.1 mg, about 36.2 mg, about 36.3 mg, about 36.4 mg, about 36.5 mg, about 36.6 mg, about 36.7 mg, about 36.8 mg, about 36.9 mg, about 37 mg, about 37.1 mg, about 37.2 mg, about 37.3 mg, about 37.4 mg, about 37.5 mg, about 37.6 mg, about 37.7 mg, about 37.8 mg, about 37.9 mg, about 38 mg, about 38.1 mg, about 38.2 mg, about 38.3 mg, about 38.4 mg, about 38.5 mg, about 38.6 mg, about 38.7 mg, about 38.8 mg, about 38.9 mg, about 39 mg, about 39.1 mg, about 39.2 mg, about 39.3 mg, about 39.4 mg, about 39.5 mg, about 39.6 mg, about 39.7 mg, about 39.8 mg, about 39.9 mg, about 40 mg, about 40.1 mg, about 40.2 mg, about 40.3 mg, about 40.4 mg, about 40.5 mg, about 40.6 mg, about 40.7 mg, about 40.8 mg, about 40.9 mg, about 41 mg, about 41.1 mg, about 41.2 mg, about 41.3 mg, about 41.4 mg, about 41.5 mg, about 41.6 mg, about 41.7 mg, about 41.8 mg, about 41.9 mg, about 42 mg, about 42.1 mg, about 42.2 mg, about 42.3 mg, about 42.4 mg, about 42.5 mg, about 42.6 mg, about 42.7 mg, about 42.8 mg, about 42.9 mg, about 43 mg, about 43.1 mg, about 43.2 mg, about 43.3 mg, about 43.4 mg, about 43.5 mg, about 43.6 mg, about 43.7 mg, about 43.8 mg, about 43.9 mg, about 44 mg, about 44.1 mg, about 44.2 mg, about 44.3 mg, about 44.4 mg, about 44.5 mg, about 44.6 mg, about 44.7 mg, about 44.8 mg, about 44.9 mg, about 45 mg, about 45.1 mg, about 45.2 mg, about 45.3 mg, about 45.4 mg, about 45.5 mg, about 45.6 mg, about 45.7 mg, about 45.8 mg, about 45.9 mg, about 46 mg, about 46.1 mg, about 46.2 mg, about 46.3 mg, about 46.4 mg, about 46.5 mg, about 46.6 mg, about 46.7 mg, about 46.8 mg, about 46.9 mg, about 47 mg, about 47.1 mg, about 47.2 mg, about 47.3 mg, about 47.4 mg, about 47.5 mg, about 47.6 mg, about 47.7 mg, about 47.8 mg, about 47.9 mg, about 48 mg, about 48.1 mg, about 48.2 mg, about 48.3 mg, about 48.4 mg, about 48.5 mg, about 48.6 mg, about 48.7 mg, about 48.8 mg, about 48.9 mg, about 49 mg, about 49.1 mg, about 49.2 mg, about 49.3 mg, about 49.4 mg, about 49.5 mg, about 49.6 mg, about 49.7 mg, about 49.8 mg, about 49.9 mg, about 50 mg, about 50.1 mg, about 50.2 mg, about 50.3 mg, about 50.4 mg, about 50.5 mg, about 50.6 mg, about 50.7 mg, about 50.8 mg, about 50.9 mg, about 51 mg, about 51.1 mg, about 51.2 mg, about 51.3 mg, about 51.4 mg, about 51.5 mg, about 51.6 mg, about 51.7 mg, about 51.8 mg, about 51.9 mg, about 52 mg, about 52.1 mg, about 52.2 mg, about 52.3 mg, about 52.4 mg, about 52.5 mg, about 52.6 mg, about 52.7 mg, about 52.8 mg, about 52.9 mg, about 53 mg, about 53.1 mg, about 53.2 mg, about 53.3 mg, about 53.4 mg, about 53.5 mg, about 53.6 mg, about 53.7 mg, about 53.8 mg, about 53.9 mg, about 54 mg, about 54.1 mg, about 54.2 mg, about 54.3 mg, about 54.4 mg, about 54.5 mg, about 54.6 mg, about 54.7 mg, about 54.8 mg, about 54.9 mg, about 55 mg, about 55.1 mg, about 55.2 mg, about 55.3 mg, about 55.4 mg, about 55.5 mg, about 55.6 mg, about 55.7 mg, about 55.8 mg, about 55.9 mg, about 56 mg, about 56.1 mg, about 56.2 mg, about 56.3 mg, about 56.4 mg, about 56.5 mg, about 56.6 mg, about 56.7 mg, about 56.8 mg, about 56.9 mg, about 57 mg, about 57.1 mg, about 57.2 mg, about 57.3 mg, about 57.4 mg, about 57.5 mg, about 57.6 mg, about 57.7 mg, about 57.8 mg, about 57.9 mg, about 58 mg, about 58.1 mg, about 58.2 mg, about 58.3 mg, about 58.4 mg, about 58.5 mg, about 58.6 mg, about 58.7 mg, about 58.8 mg, about 58.9 mg, about 59 mg, about 59.1 mg, about 59.2 mg, about 59.3 mg, about 59.4 mg, about 59.5 mg, about 59.6 mg, about 59.7 mg, about 59.8 mg, about 59.9 mg, about 60 mg, about 60.1 mg, about 60.2 mg, about 60.3 mg, about 60.4 mg, about 60.5 mg, about 60.6 mg, about 60.7 mg, about 60.8 mg, about 60.9 mg, about 61 mg, about 61.1 mg, about 61.2 mg, about 61.3 mg, about 61.4 mg, about 61.5 mg, about 61.6 mg, about 61.7 mg, about 61.8 mg, about 61.9 mg, about 62 mg, about 62.1 mg, about 62.2 mg, about 62.3 mg, about 62.4 mg, about 62.5 mg, about 62.6 mg, about 62.7 mg, about 62.8 mg, about 62.9 mg, about 63 mg, about 63.1 mg, about 63.2 mg, about 63.3 mg, about 63.4 mg, about 63.5 mg, about 63.6 mg, about 63.7 mg, about 63.8 mg, about 63.9 mg, about 64 mg, about 64.1 mg, about 64.2 mg, about 64.3 mg, about 64.4 mg, about 64.5 mg, about 64.6 mg, about 64.7 mg, about 64.8 mg, about 64.9 mg, about 65 mg, about 65.1 mg, about 65.2 mg, about 65.3 mg, about 65.4 mg, about 65.5 mg, about 65.6 mg, about 65.7 mg, about 65.8 mg, about 65.9 mg, about 66 mg, about 66.1 mg, about 66.2 mg, about 66.3 mg, about 66.4 mg, about 66.5 mg, about 66.6 mg, about 66.7 mg, about 66.8 mg, about 66.9 mg, about 67 mg, about 67.1 mg, about 67.2 mg, about 67.3 mg, about 67.4 mg, about 67.5 mg, about 67.6 mg, about 67.7 mg, about 67.8 mg, about 67.9 mg, about 68 mg, about 68.1 mg, about 68.2 mg, about 68.3 mg, about 68.4 mg, about 68.5 mg, about 68.6 mg, about 68.7 mg, about 68.8 mg, about 68.9 mg, about 69 mg, about 69.1 mg, about 69.2 mg, about 69.3 mg, about 69.4 mg, about 69.5 mg, about 69.6 mg, about 69.7 mg, about 69.8 mg, and about 69.9 mg.

In certain embodiments, the therapeutically effective amount is any of 70 mg, 70.1 mg, 70.2 mg, 70.3 mg, 70.4 mg, 70.5 mg, 70.6 mg, 70.7 mg, 70.8 mg, 70.9 mg, 71 mg, 71.1 mg, 71.2 mg, 71.3 mg, 71.4 mg, 71.5 mg, 71.6 mg, 71.7 mg, 71.8 mg, 71.9 mg, 72 mg, 72.1 mg, 72.2 mg, 72.3 mg, 72.4 mg, 72.5 mg, 72.6 mg, 72.7 mg, 72.8 mg, 72.9 mg, 73 mg, 73.1 mg, 73.2 mg, 73.3 mg, 73.4 mg, 73.5 mg, 73.6 mg, 73.7 mg, 73.8 mg, 73.9 mg, 74 mg, 74.1 mg, 74.2 mg, 74.3 mg, 74.4 mg, 74.5 mg, 74.6 mg, 74.7 mg, 74.8 mg, 74.9 mg, 75 mg, 75.1 mg, 75.2 mg, 75.3 mg, 75.4 mg, 75.5 mg, 75.6 mg, 75.7 mg, 75.8 mg, 75.9 mg, 76 mg, 76.1 mg, 76.2 mg, 76.3 mg, 76.4 mg, 76.5 mg, 76.6 mg, 76.7 mg, 76.8 mg, 76.9 mg, 77 mg, 77.1 mg, 77.2 mg, 77.3 mg, 77.4 mg, 77.5 mg, 77.6 mg, 77.7 mg, 77.8 mg, 77.9 mg, 78 mg, 78.1 mg, 78.2 mg, 78.3 mg, 78.4 mg, 78.5 mg, 78.6 mg, 78.7 mg, 78.8 mg, 78.9 mg, 79 mg, 79.1 mg, 79.2 mg, 79.3 mg, 79.4 mg, 79.5 mg, 79.6 mg, 79.7 mg, 79.8 mg, 79.9 mg, 80 mg, 80.1 mg, 80.2 mg, 80.3 mg, 80.4 mg, 80.5 mg, 80.6 mg, 80.7 mg, 80.8 mg, 80.9 mg, 81 mg, 81.1 mg, 81.2 mg, 81.3 mg, 81.4 mg, 81.5 mg, 81.6 mg, 81.7 mg, 81.8 mg, 81.9 mg, 82 mg, 82.1 mg, 82.2 mg, 82.3 mg, 82.4 mg, 82.5 mg, 82.6 mg, 82.7 mg, 82.8 mg, 82.9 mg, 83 mg, 83.1 mg, 83.2 mg, 83.3 mg, 83.4 mg, 83.5 mg, 83.6 mg, 83.7 mg, 83.8 mg, 83.9 mg, 84 mg, 84.1 mg, 84.2 mg, 84.3 mg, 84.4 mg, 84.5 mg, 84.6 mg, 84.7 mg, 84.8 mg, 84.9 mg, 85 mg, 85.1 mg, 85.2 mg, 85.3 mg, 85.4 mg, 85.5 mg, 85.6 mg, 85.7 mg, 85.8 mg, 85.9 mg, 86 mg, 86.1 mg, 86.2 mg, 86.3 mg, 86.4 mg, 86.5 mg, 86.6 mg, 86.7 mg, 86.8 mg, 86.9 mg, 87 mg, 87.1 mg, 87.2 mg, 87.3 mg, 87.4 mg, 87.5 mg, 87.6 mg, 87.7 mg, 87.8 mg, 87.9 mg, 88 mg, 88.1 mg, 88.2 mg, 88.3 mg, 88.4 mg, 88.5 mg, 88.6 mg, 88.7 mg, 88.8 mg, 88.9 mg, 89 mg, 89.1 mg, 89.2 mg, 89.3 mg, 89.4 mg, 89.5 mg, 89.6 mg, 89.7 mg, 89.8 mg, 89.9 mg, 90 mg, 90.1 mg, 90.2 mg, 90.3 mg, 90.4 mg, 90.5 mg, 90.6 mg, 90.7 mg, 90.8 mg, 90.9 mg, 91 mg, 91.1 mg, 91.2 mg, 91.3 mg, 91.4 mg, 91.5 mg, 91.6 mg, 91.7 mg, 91.8 mg, 91.9 mg, 92 mg, 92.1 mg, 92.2 mg, 92.3 mg, 92.4 mg, 92.5 mg, 92.6 mg, 92.7 mg, 92.8 mg, 92.9 mg, 93 mg, 93.1 mg, 93.2 mg, 93.3 mg, 93.4 mg, 93.5 mg, 93.6 mg, 93.7 mg, 93.8 mg, 93.9 mg, 94 mg, 94.1 mg, 94.2 mg, 94.3 mg, 94.4 mg, 94.5 mg, 94.6 mg, 94.7 mg, 94.8 mg, 94.9 mg, 95 mg, 95.1 mg, 95.2 mg, 95.3 mg, 95.4 mg, 95.5 mg, 95.6 mg, 95.7 mg, 95.8 mg, 95.9 mg, 96 mg, 96.1 mg, 96.2 mg, 96.3 mg, 96.4 mg, 96.5 mg, 96.6 mg, 96.7 mg, 96.8 mg, 96.9 mg, 97 mg, 97.1 mg, 97.2 mg, 97.3 mg, 97.4 mg, 97.5 mg, 97.6 mg, 97.7 mg, 97.8 mg, 97.9 mg, 98 mg, 98.1 mg, 98.2 mg, 98.3 mg, 98.4 mg, 98.5 mg, 98.6 mg, 98.7 mg, 98.8 mg, 98.9 mg, 99 mg, 99.1 mg, 99.2 mg, 99.3 mg, 99.4 mg, 99.5 mg, 99.6 mg, 99.7 mg, 99.8 mg, and 99.9 mg.

In certain embodiments, the therapeutically effective amount is any of about 70 mg, about 70.1 mg, about 70.2 mg, about 70.3 mg, about 70.4 mg, about 70.5 mg, about 70.6 mg, about 70.7 mg, about 70.8 mg, about 70.9 mg, about 71 mg, about 71.1 mg, about 71.2 mg, about 71.3 mg, about 71.4 mg, about 71.5 mg, about 71.6 mg, about 71.7 mg, about 71.8 mg, about 71.9 mg, about 72 mg, about 72.1 mg, about 72.2 mg, about 72.3 mg, about 72.4 mg, about 72.5 mg, about 72.6 mg, about 72.7 mg, about 72.8 mg, about 72.9 mg, about 73 mg, about 73.1 mg, about 73.2 mg, about 73.3 mg, about 73.4 mg, about 73.5 mg, about 73.6 mg, about 73.7 mg, about 73.8 mg, about 73.9 mg, about 74 mg, about 74.1 mg, about 74.2 mg, about 74.3 mg, about 74.4 mg, about 74.5 mg, about 74.6 mg, about 74.7 mg, about 74.8 mg, about 74.9 mg, about 75 mg, about 75.1 mg, about 75.2 mg, about 75.3 mg, about 75.4 mg, about 75.5 mg, about 75.6 mg, about 75.7 mg, about 75.8 mg, about 75.9 mg, about 76 mg, about 76.1 mg, about 76.2 mg, about 76.3 mg, about 76.4 mg, about 76.5 mg, about 76.6 mg, about 76.7 mg, about 76.8 mg, about 76.9 mg, about 77 mg, about 77.1 mg, about 77.2 mg, about 77.3 mg, about 77.4 mg, about 77.5 mg, about 77.6 mg, about 77.7 mg, about 77.8 mg, about 77.9 mg, about 78 mg, about 78.1 mg, about 78.2 mg, about 78.3 mg, about 78.4 mg, about 78.5 mg, about 78.6 mg, about 78.7 mg, about 78.8 mg, about 78.9 mg, about 79 mg, about 79.1 mg, about 79.2 mg, about 79.3 mg, about 79.4 mg, about 79.5 mg, about 79.6 mg, about 79.7 mg, about 79.8 mg, about 79.9 mg, about 80 mg, about 80.1 mg, about 80.2 mg, about 80.3 mg, about 80.4 mg, about 80.5 mg, about 80.6 mg, about 80.7 mg, about 80.8 mg, about 80.9 mg, about 81 mg, about 81.1 mg, about 81.2 mg, about 81.3 mg, about 81.4 mg, about 81.5 mg, about 81.6 mg, about 81.7 mg, about 81.8 mg, about 81.9 mg, about 82 mg, about 82.1 mg, about 82.2 mg, about 82.3 mg, about 82.4 mg, about 82.5 mg, about 82.6 mg, about 82.7 mg, about 82.8 mg, about 82.9 mg, about 83 mg, about 83.1 mg, about 83.2 mg, about 83.3 mg, about 83.4 mg, about 83.5 mg, about 83.6 mg, about 83.7 mg, about 83.8 mg, about 83.9 mg, about 84 mg, about 84.1 mg, about 84.2 mg, about 84.3 mg, about 84.4 mg, about 84.5 mg, about 84.6 mg, about 84.7 mg, about 84.8 mg, about 84.9 mg, about 85 mg, about 85.1 mg, about 85.2 mg, about 85.3 mg, about 85.4 mg, about 85.5 mg, about 85.6 mg, about 85.7 mg, about 85.8 mg, about 85.9 mg, about 86 mg, about 86.1 mg, about 86.2 mg, about 86.3 mg, about 86.4 mg, about 86.5 mg, about 86.6 mg, about 86.7 mg, about 86.8 mg, about 86.9 mg, about 87 mg, about 87.1 mg, about 87.2 mg, about 87.3 mg, about 87.4 mg, about 87.5 mg, about 87.6 mg, about 87.7 mg, about 87.8 mg, about 87.9 mg, about 88 mg, about 88.1 mg, about 88.2 mg, about 88.3 mg, about 88.4 mg, about 88.5 mg, about 88.6 mg, about 88.7 mg, about 88.8 mg, about 88.9 mg, about 89 mg, about 89.1 mg, about 89.2 mg, about 89.3 mg, about 89.4 mg, about 89.5 mg, about 89.6 mg, about 89.7 mg, about 89.8 mg, about 89.9 mg, about 90 mg, about 90.1 mg, about 90.2 mg, about 90.3 mg, about 90.4 mg, about 90.5 mg, about 90.6 mg, about 90.7 mg, about 90.8 mg, about 90.9 mg, about 91 mg, about 91.1 mg, about 91.2 mg, about 91.3 mg, about 91.4 mg, about 91.5 mg, about 91.6 mg, about 91.7 mg, about 91.8 mg, about 91.9 mg, about 92 mg, about 92.1 mg, about 92.2 mg, about 92.3 mg, about 92.4 mg, about 92.5 mg, about 92.6 mg, about 92.7 mg, about 92.8 mg, about 92.9 mg, about 93 mg, about 93.1 mg, about 93.2 mg, about 93.3 mg, about 93.4 mg, about 93.5 mg, about 93.6 mg, about 93.7 mg, about 93.8 mg, about 93.9 mg, about 94 mg, about 94.1 mg, about 94.2 mg, about 94.3 mg, about 94.4 mg, about 94.5 mg, about 94.6 mg, about 94.7 mg, about 94.8 mg, about 94.9 mg, about 95 mg, about 95.1 mg, about 95.2 mg, about 95.3 mg, about 95.4 mg, about 95.5 mg, about 95.6 mg, about 95.7 mg, about 95.8 mg, about 95.9 mg, about 96 mg, about 96.1 mg, about 96.2 mg, about 96.3 mg, about 96.4 mg, about 96.5 mg, about 96.6 mg, about 96.7 mg, about 96.8 mg, about 96.9 mg, about 97 mg, about 97.1 mg, about 97.2 mg, about 97.3 mg, about 97.4 mg, about 97.5 mg, about 97.6 mg, about 97.7 mg, about 97.8 mg, about 97.9 mg, about 98 mg, about 98.1 mg, about 98.2 mg, about 98.3 mg, about 98.4 mg, about 98.5 mg, about 98.6 mg, about 98.7 mg, about 98.8 mg, about 98.9 mg, about 99 mg, about 99.1 mg, about 99.2 mg, about 99.3 mg, about 99.4 mg, about 99.5 mg, about 99.6 mg, about 99.7 mg, about 99.8 mg, and about 99.9 mg.

In certain embodiments, the therapeutically effective amount is any of 100 mg, 100.1 mg, 100.2 mg, 100.3 mg, 100.4 mg, 100.5 mg, 100.6 mg, 100.7 mg, 100.8 mg, 100.9 mg, 101 mg, 101.1 mg, 101.2 mg, 101.3 mg, 101.4 mg, 101.5 mg, 101.6 mg, 101.7 mg, 101.8 mg, 101.9 mg, 102 mg, 102.1 mg, 102.2 mg, 102.3 mg, 102.4 mg, 102.5 mg, 102.6 mg, 102.7 mg, 102.8 mg, 102.9 mg, 103 mg, 103.1 mg, 103.2 mg, 103.3 mg, 103.4 mg, 103.5 mg, 103.6 mg, 103.7 mg, 103.8 mg, 103.9 mg, 104 mg, 104.1 mg, 104.2 mg, 104.3 mg, 104.4 mg, 104.5 mg, 104.6 mg, 104.7 mg, 104.8 mg, 104.9 mg, 105 mg, 105.1 mg, 105.2 mg, 105.3 mg, 105.4 mg, 105.5 mg, 105.6 mg, 105.7 mg, 105.8 mg, 105.9 mg, 106 mg, 106.1 mg, 106.2 mg, 106.3 mg, 106.4 mg, 106.5 mg, 106.6 mg, 106.7 mg, 106.8 mg, 106.9 mg, 107 mg, 107.1 mg, 107.2 mg, 107.3 mg, 107.4 mg, 107.5 mg, 107.6 mg, 107.7 mg, 107.8 mg, 107.9 mg, 108 mg, 108.1 mg, 108.2 mg, 108.3 mg, 108.4 mg, 108.5 mg, 108.6 mg, 108.7 mg, 108.8 mg, 108.9 mg, 109 mg, 109.1 mg, 109.2 mg, 109.3 mg, 109.4 mg, 109.5 mg, 109.6 mg, 109.7 mg, 109.8 mg, 109.9 mg, 110 mg, 110.1 mg, 110.2 mg, 110.3 mg, 110.4 mg, 110.5 mg, 110.6 mg, 110.7 mg, 110.8 mg, 110.9 mg, 111 mg, 111.1 mg, 111.2 mg, 111.3 mg, 111.4 mg, 111.5 mg, 111.6 mg, 111.7 mg, 111.8 mg, 111.9 mg, 112 mg, 112.1 mg, 112.2 mg, 112.3 mg, 112.4 mg, 112.5 mg, 112.6 mg, 112.7 mg, 112.8 mg, 112.9 mg, 113 mg, 113.1 mg, 113.2 mg, 113.3 mg, 113.4 mg, 113.5 mg, 113.6 mg, 113.7 mg, 113.8 mg, 113.9 mg, 114 mg, 114.1 mg, 114.2 mg, 114.3 mg, 114.4 mg, 114.5 mg, 114.6 mg, 114.7 mg, 114.8 mg, 114.9 mg, 115 mg, 115.1 mg, 115.2 mg, 115.3 mg, 115.4 mg, 115.5 mg, 115.6 mg, 115.7 mg, 115.8 mg, 115.9 mg, 116 mg, 116.1 mg, 116.2 mg, 116.3 mg, 116.4 mg, 116.5 mg, 116.6 mg, 116.7 mg, 116.8 mg, 116.9 mg, 117 mg, 117.1 mg, 117.2 mg, 117.3 mg, 117.4 mg, 117.5 mg, 117.6 mg, 117.7 mg, 117.8 mg, 117.9 mg, 118 mg, 118.1 mg, 118.2 mg, 118.3 mg, 118.4 mg, 118.5 mg, 118.6 mg, 118.7 mg, 118.8 mg, 118.9 mg, 119 mg, 119.1 mg, 119.2 mg, 119.3 mg, 119.4 mg, 119.5 mg, 119.6 mg, 119.7 mg, 119.8 mg, 119.9 mg, 120 mg, 120.1 mg, 120.2 mg, 120.3 mg, 120.4 mg, 120.5 mg, 120.6 mg, 120.7 mg, 120.8 mg, 120.9 mg, 121 mg, 121.1 mg, 121.2 mg, 121.3 mg, 121.4 mg, 121.5 mg, 121.6 mg, 121.7 mg, 121.8 mg, 121.9 mg, 122 mg, 122.1 mg, 122.2 mg, 122.3 mg, 122.4 mg, 122.5 mg, 122.6 mg, 122.7 mg, 122.8 mg, 122.9 mg, 123 mg, 123.1 mg, 123.2 mg, 123.3 mg, 123.4 mg, 123.5 mg, 123.6 mg, 123.7 mg, 123.8 mg, 123.9 mg, 124 mg, 124.1 mg, 124.2 mg, 124.3 mg, 124.4 mg, 124.5 mg, 124.6 mg, 124.7 mg, 124.8 mg, 124.9 mg, and 125 mg.

In certain embodiments, the therapeutically effective amount is any of about 100 mg, about 100.1 mg, about 100.2 mg, about 100.3 mg, about 100.4 mg, about 100.5 mg, about 100.6 mg, about 100.7 mg, about 100.8 mg, about 100.9 mg, about 101 mg, about 101.1 mg, about 101.2 mg, about 101.3 mg, about 101.4 mg, about 101.5 mg, about 101.6 mg, about 101.7 mg, about 101.8 mg, about 101.9 mg, about 102 mg, about 102.1 mg, about 102.2 mg, about 102.3 mg, about 102.4 mg, about 102.5 mg, about 102.6 mg, about 102.7 mg, about 102.8 mg, about 102.9 mg, about 103 mg, about 103.1 mg, about 103.2 mg, about 103.3 mg, about 103.4 mg, about 103.5 mg, about 103.6 mg, about 103.7 mg, about 103.8 mg, about 103.9 mg, about 104 mg, about 104.1 mg, about 104.2 mg, about 104.3 mg, about 104.4 mg, about 104.5 mg, about 104.6 mg, about 104.7 mg, about 104.8 mg, about 104.9 mg, about 105 mg, about 105.1 mg, about 105.2 mg, about 105.3 mg, about 105.4 mg, about 105.5 mg, about 105.6 mg, about 105.7 mg, about 105.8 mg, about 105.9 mg, about 106 mg, about 106.1 mg, about 106.2 mg, about 106.3 mg, about 106.4 mg, about 106.5 mg, about 106.6 mg, about 106.7 mg, about 106.8 mg, about 106.9 mg, about 107 mg, about 107.1 mg, about 107.2 mg, about 107.3 mg, about 107.4 mg, about 107.5 mg, about 107.6 mg, about 107.7 mg, about 107.8 mg, about 107.9 mg, about 108 mg, about 108.1 mg, about 108.2 mg, about 108.3 mg, about 108.4 mg, about 108.5 mg, about 108.6 mg, about 108.7 mg, about 108.8 mg, about 108.9 mg, about 109 mg, about 109.1 mg, about 109.2 mg, about 109.3 mg, about 109.4 mg, about 109.5 mg, about 109.6 mg, about 109.7 mg, about 109.8 mg, about 109.9 mg, about 110 mg, about 110.1 mg, about 110.2 mg, about 110.3 mg, about 110.4 mg, about 110.5 mg, about 110.6 mg, about 110.7 mg, about 110.8 mg, about 110.9 mg, about 111 mg, about 111.1 mg, about 111.2 mg, about 111.3 mg, about 111.4 mg, about 111.5 mg, about 111.6 mg, about 111.7 mg, about 111.8 mg, about 111.9 mg, about 112 mg, about 112.1 mg, about 112.2 mg, about 112.3 mg, about 112.4 mg, about 112.5 mg, about 112.6 mg, about 112.7 mg, about 112.8 mg, about 112.9 mg, about 113 mg, about 113.1 mg, about 113.2 mg, about 113.3 mg, about 113.4 mg, about 113.5 mg, about 113.6 mg, about 113.7 mg, about 113.8 mg, about 113.9 mg, about 114 mg, about 114.1 mg, about 114.2 mg, about 114.3 mg, about 114.4 mg, about 114.5 mg, about 114.6 mg, about 114.7 mg, about 114.8 mg, about 114.9 mg, about 115 mg, about 115.1 mg, about 115.2 mg, about 115.3 mg, about 115.4 mg, about 115.5 mg, about 115.6 mg, about 115.7 mg, about 115.8 mg, about 115.9 mg, about 116 mg, about 116.1 mg, about 116.2 mg, about 116.3 mg, about 116.4 mg, about 116.5 mg, about 116.6 mg, about 116.7 mg, about 116.8 mg, about 116.9 mg, about 117 mg, about 117.1 mg, about 117.2 mg, about 117.3 mg, about 117.4 mg, about 117.5 mg, about 117.6 mg, about 117.7 mg, about 117.8 mg, about 117.9 mg, about 118 mg, about 118.1 mg, about 118.2 mg, about 118.3 mg, about 118.4 mg, about 118.5 mg, about 118.6 mg, about 118.7 mg, about 118.8 mg, about 118.9 mg, about 119 mg, about 119.1 mg, about 119.2 mg, about 119.3 mg, about 119.4 mg, about 119.5 mg, about 119.6 mg, about 119.7 mg, about 119.8 mg, about 119.9 mg, about 120 mg, about 120.1 mg, about 120.2 mg, about 120.3 mg, about 120.4 mg, about 120.5 mg, about 120.6 mg, about 120.7 mg, about 120.8 mg, about 120.9 mg, about 121 mg, about 121.1 mg, about 121.2 mg, about 121.3 mg, about 121.4 mg, about 121.5 mg, about 121.6 mg, about 121.7 mg, about 121.8 mg, about 121.9 mg, about 122 mg, about 122.1 mg, about 122.2 mg, about 122.3 mg, about 122.4 mg, about 122.5 mg, about 122.6 mg, about 122.7 mg, about 122.8 mg, about 122.9 mg, about 123 mg, about 123.1 mg, about 123.2 mg, about 123.3 mg, about 123.4 mg, about 123.5 mg, about 123.6 mg, about 123.7 mg, about 123.8 mg, about 123.9 mg, about 124 mg, about 124.1 mg, about 124.2 mg, about 124.3 mg, about 124.4 mg, about 124.5 mg, about 124.6 mg, about 124.7 mg, about 124.8 mg, about 124.9 mg, and about 125 mg.

In certain embodiments, the therapeutically effective amount is any of 10 mg to 200 mg, 10 mg to 190 mg, 10 mg to 180 mg, 10 mg to 170 mg, from 10 mg to 160 mg, 10 mg to 150 mg, 10 mg to 140 mg, 10 mg to 120 mg, 10 mg to 110 mg, 10 mg to 100 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 200 mg, 20 mg to 190 mg, 20 mg to 180 mg, 20 mg to 170 mg, from 20 mg to 160 mg, 20 mg to 150 mg, 20 mg to 140 mg, 20 mg to 120 mg, 20 mg to 110 mg, 20 mg to 100 mg, 20 mg to 80 mg, 20 mg to 70 mg, 20 mg to 60 mg, 20 mg to 50 mg, 20 mg to 10 mg, 20 mg to 30 mg, 30 mg to 200 mg, 30 mg to 190 mg, 30 mg to 180 mg, 30 mg to 170 mg, from 30 mg to 160 mg, 30 mg to 150 mg, 30 mg to 140 mg, 30 mg to 120 mg, 30 mg to 110 mg, 30 mg to 100 mg, 30 mg to 80 mg, 30 mg to 70 mg, 30 mg to 60 mg, 30 mg to 50 mg, 30 mg to 20 mg, 40 mg to 200 mg, 40 mg to 190 mg, 40 mg to 180 mg, 40 mg to 170 mg, 40 mg to 160 mg, 40 mg to 150 mg, 40 mg to 140 mg, 40 mg to 120 mg, 40 mg to 110 mg, 40 mg to 100 mg, 40 mg to 80 mg, 40 mg to 70 mg, 40 mg to 60 mg, 40 mg to 50 mg, 50 mg to 200 mg, 50 mg to 190 mg, 50 mg to 180 mg, 50 mg to 170 mg, 50 mg to 160 mg, 50 mg to 150 mg, 50 mg to 140 mg, 50 mg to 120 mg, 50 mg to 110 mg, 50 mg to 100 mg, 50 mg to 80 mg, 50 mg to 70 mg, 50 mg to 60 mg, 60 mg to 200 mg, 60 mg to 190 mg, 60 mg to 180 mg, 60 mg to 170 mg, 60 mg to 160 mg, 60 mg to 150 mg, 60 mg to 140 mg, 60 mg to 120 mg, 60 mg to 115 mg, 60 mg to 110 mg, 60 mg to 100 mg, 60 mg to 80 mg, 60 mg to 70 mg, 70 mg to 200 mg, 70 mg to 190 mg, 70 mg to 180 mg, 70 mg to 170 mg, 70 mg to 160 mg, 70 mg to 150 mg, 70 mg to 140 mg, 70 mg to 120 mg, 70 mg to 110 mg, 70 mg to 100 mg, 70 mg to 80 mg, 80 mg to 200 mg, 80 mg to 190 mg, 80 mg to 180 mg, 80 mg to 170 mg, 80 mg to 160 mg, 80 mg to 150 mg, 80 mg to 140 mg, 80 mg to 120 mg, 80 mg to 110 mg, 80 mg to 100 mg, 80 mg to 90 mg, 90 mg to 200 mg, 90 mg to 190 mg, 90 mg to 180 mg, 90 mg to 170 mg, 90 mg to 160 mg, 90 mg to 150 mg, 90 mg to 140 mg, 90 mg to 120 mg, 90 mg to 110 mg, 90 mg to 100 mg, 100 mg to 200 mg, 100 mg to 190 mg, 100 mg to 180 mg, 100 mg to 170 mg, 100 mg to 160 mg, 100 mg to 150 mg, 100 mg to 140 mg, 100 mg to 120 mg, 100 mg to 110 mg, 110 mg to 200 mg, 110 mg to 190 mg, 110 mg to 180 mg, 110 mg to 170 mg, 110 mg to 160 mg, 110 mg to 150 mg, 110 mg to 140 mg, 110 mg to 130 mg, 110 mg to 120 mg, 120 mg to 200 mg, 120 mg to 190 mg, 120 mg to 180 mg, 120 mg to 170 mg, 120 mg to 160 mg, 120 mg to 150 mg, 120 mg to 140 mg, 120 mg to 130 mg, 130 mg to 200 mg, 130 mg to 190 mg, 130 mg to 180 mg, 130 mg to 170 mg, 130 mg to 160 mg, 130 mg to 150 mg, 130 mg to 140 mg, 140 mg to 200 mg, 140 mg to 190 mg, 140 mg to 180 mg, 140 mg to 170 mg, 140 mg to 160 mg, 140 mg to 150 mg, 150 mg to 200 mg, 150 mg to 190 mg, 150 mg to 180 mg, 150 mg to 170 mg, 150 mg to 160 mg, 160 mg to 200 mg, 160 mg to 190 mg, 160 mg to 180 mg, 160 mg to 170 mg, 180 mg to 200 mg, 180 mg to 190 mg, 190 mg to 200 mg, 105 mg to 135 mg, 105 mg to 130 mg, 105 mg to 125 mg 105 mg to 120 mg, 110 mg to 135 mg, 110 mg to 130 mg, 110 mg to 125 mg, 110 mg to 120 mg, 115 mg to 135 mg, 115 mg to 130 mg, 115 mg to 125 mg, 115 mg to 120 mg, 115 mg to 125 mg, 115 mg to 120 mg, 120 mg to 135 mg, 120 mg to 125 mg, 125 mg to 140 mg, 125 mg to 130 mg, 130 mg to 135 mg, 135 mg to 140 mg, 120 mg to 129 mg, 120 mg to 128 mg, 120 mg to 127 mg, 120 mg to 86 mg, 120 mg to 124 mg, 120 mg to 123 mg, 120 mg to 122 mg, 120 mg to 121 mg, 121 mg to 130 mg, 122 mg to 129 mg, 122 mg to 128 mg, 122 mg to 127 mg, 122 mg to 126 mg, 122 mg to 125 mg, 122 mg to 124 mg, 122 mg to 123 mg, 123 mg to 130 mg, 123 mg to 129 mg, 123 mg to 128 mg, 123 mg to 127 mg, 123 mg to 126 mg, 123 mg to 125 mg, 123 mg to 124 mg, 124 mg to 130 mg, 124 mg to 129 mg, 124 mg to 128 mg, 124 mg to 127 mg, 124 mg to 126 mg, 124 mg to 125 mg, 125 mg to 129 mg, 125 mg to 128 mg, 125 mg to 127 mg, 125 mg to 126 mg, 126 mg to 130 mg, 126 mg to 129 mg, 126 mg to 128 mg, 126 mg to 127 mg, 127 mg to 130 mg, 127 mg to 129 mg, 127 mg to 128 mg, 128 mg to 130 mg, 128 mg to 129 mg, and 129 mg to 130 mg.

In certain embodiments, the therapeutically effective amount is any of less than 350 mg, less than 345 mg, less than 340 mg, less than 335 mg, less than 330 mg, less than 325 mg, less than 320 mg, less than 315 mg, less than 310 mg, less than 305 mg, less than 300 mg, less than 295 mg, less than 290 mg, less than 285 mg, less than 280 mg, less than 275 mg, less than 270 mg, less than 265 mg, less than 260 mg, less than 255 mg, less than 250 mg, less than 245 mg, less than 240 mg, less than 235 mg, less than 230 mg, less than 225 mg, less than 220 mg, less than 215 mg, less than 210 mg, less than 205 mg, less than 200 mg, less than 195 mg, less than 190 mg, less than 185 mg, less than 180 mg, less than 175 mg, less than 170 mg, less than 165 mg, less than 160 mg, less than 150 mg, less than 145 mg, less than 140 mg, less than 135 mg, less than 130 mg, less than 125 mg, less than 120 mg, less than 115 mg, less than 110 mg, less than 105 mg, less than 100 mg, less than 95 mg, less than 90 mg, less than 85 mg, less than 80 mg, less than 75 mg, less than 70 mg, less than 65 mg, less than 60 mg, less than 55 mg, less than 50 mg, less than 45 mg, less than 40 mg, less than 35 mg, less than 30 mg, less than 25 mg, less than 20 mg, less than 15 mg, less than 10 mg, and less than 5 mg.

In certain embodiments, the therapeutically effective amount is any of less than about 350 mg, less than about 345 mg, less than about 340 mg, less than about 335 mg, less than about 330 mg, less than about 325 mg, less than about 320 mg, less than about 315 mg, less than about 310 mg, less than about 305 mg, less than about 300 mg, less than about 295 mg, less than about 290 mg, less than about 285 mg, less than about 280 mg, less than about 275 mg, less than about 270 mg, less than about 265 mg, less than about 260 mg, less than about 255 mg, less than about 250 mg, less than about 245 mg, less than about 240 mg, less than about 235 mg, less than about 230 mg, less than about 225 mg, less than about 220 mg, less than about 215 mg, less than about 210 mg, less than about 205 mg, less than about 200 mg, less than about 195 mg, less than about 190 mg, less than about 185 mg, less than about 180 mg, less than about 175 mg, less than about 170 mg, less than about 165 mg, less than about 160 mg, less than about 150 mg, less than about 145 mg, less than about 140 mg, less than about 135 mg, less than about 130 mg, less than about 125 mg, less than about 120 mg, less than about 115 mg, less than about 110 mg, less than about 105 mg, less than about 100 mg, less than about 95 mg, less than about 90 mg, less than about 85 mg, less than about 80 mg, less than about 75 mg, less than about 70 mg, less than about 65 mg, less than about 60 mg, less than about 55 mg, less than about 50 mg, less than about 45 mg, less than about 40 mg, less than about 35 mg, less than about 30 mg, less than about 25 mg, less than about 20 mg, less than about 15 mg, less than about 10 mg, and less than about 5 mg.

In certain embodiments, the therapeutically effective amount is any of at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185, at least 190 mg, at least 195 mg, and at least 200 mg.

In certain embodiments, the therapeutically effective amount is any of at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 115 mg, at least about 120 mg, at least about 125 mg, at least about 130 mg, at least about 135 mg, at least about 140 mg, at least about 145 mg, or at least about 150 mg, at least about 155 mg, at least about 160 mg, at least about 165 mg, at least about 170 mg, at least about 175 mg, at least about 180 mg, at least about 185, at least about 190 mg, at least about 195 mg, and at least about 200 mg.

### V. Certain Dosing Regimens

In certain embodiments, described herein are methods of administering to a subject a therapeutically effective amount of the Compound 696844 one or more times. In certain embodiments, methods comprise administering the therapeutically effective amount at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 50, 61, 62, 63, 64, 65, 66, 67, 68, 69, or 70 times. In certain embodiments, methods comprise administering the therapeutically effective amount once every week. In certain embodiments, methods comprise administering the therapeutically effective amount once every 2 weeks. In certain embodiments, methods comprise administering the therapeutically effective amount once every 3 weeks. In certain embodiments, methods comprise administering the therapeutically effective amount once every 4 weeks. In certain embodiments, methods comprise administering the therapeutically effective amount once every 8 weeks. In certain embodiments, methods comprise administering the therapeutically effective amount once every 12 weeks. In certain embodiments, methods comprise administering the therapeutically effective amount once every 16 weeks. In certain embodiments, methods comprise administering the therapeutically effective amount once every 20 weeks. In certain embodiments, methods comprise administering the therapeutically effective amount once every 24 weeks. In certain embodiments, methods comprise administering the therapeutically effective amount once every 6 months. In certain embodiments, methods comprise administering the therapeutically effective amount monthly. In certain embodiments, methods comprise administering the therapeutically effective amount once every two months. In certain embodiments, methods comprise administering the therapeutically effective amount once every three months. In certain embodiments, methods comprise administering the therapeutically effective amount quarterly. In certain embodiments, methods comprise administering the therapeutically effective amount semiannually. In certain embodiments, methods comprise administering the therapeutically effective amount annually. In certain embodiments, methods comprise administering the therapeutically effective amount once every two years.

In certain embodiments, methods comprise administering the therapeutically effective amount about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, about every 20 weeks, about every 21 weeks, about every 22 weeks, about every 23 weeks, about every 24 weeks, or about every 6 months. In certain embodiments, methods comprise administering the therapeutically effective amount about monthly. In certain embodiments, methods comprise administering the therapeutically effective amount once about every two months. In certain embodiments, methods comprise administering the therapeutically effective amount once about every three months. In certain embodiments, methods comprise administering the therapeutically effective amount about quarterly. In certain embodiments, methods comprise administering the therapeutically effective amount about semiannually. In certain embodiments, methods comprise administering the therapeutically effective amount about annually. In certain embodiments, methods comprise administering the therapeutically effective amount once about every two years.

In certain embodiments, methods comprise administering the therapeutically effective amount at a therapeutically effective frequency for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months.

### Loading and Maintenance Doses

In certain embodiment, the therapeutically effective amount is administered as a loading dose and/or a maintenance dose. In certain embodiments, a loading dose is a different dose level than a maintenance dose. In certain embodiments, the loading dose(s) are a greater dose level than the maintenance dose(s). In certain embodiments, the loading dose(s) and maintenance dose(s) are the same dose level. In certain embodiments, methods comprise administering a loading dose or doses and subsequently administering a maintenance dose or doses. In certain embodiments, methods comprise administering a loading dose once about every 4 weeks, and subsequently administering a maintenance dose once about every 4 weeks, about every 8 weeks, about every 12 weeks, about every 16 weeks, about 20 weeks, about 24 weeks, or about 6 months. In certain embodiments, methods comprise administering a loading dose once about every 4 weeks, and subsequently administering a maintenance dose once about every 6 months. In certain embodiments, methods comprise administering a loading dose once about every 12 weeks, and subsequently administering a maintenance dose once about every 6 months.

In certain embodiments, methods comprise administering at least 2 loading doses, at least 3 loading doses, at least 4 loading doses, at least 5 loading doses, or at least 6 loading doses. In certain embodiments, methods comprise administering 2, 3, 4, 5, 6, 7, 8, 9, 10, 11, 12, 13, 14, 15, or 16 loading doses. In certain embodiments, methods comprise administering a loading dose or doses about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, or about every 12 weeks. In certain embodiments, methods comprise administering an initial loading dose and administering a second loading dose about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks after administering the initial loading dose.

In certain embodiments, methods comprise administering at least 2 maintenance doses, at least 3 maintenance doses, at least 4 maintenance doses, at least 5 maintenance doses, or at least 6 maintenance doses. In certain embodiments, methods comprise administering 2, 3, 4, 5, 6, 7, 8, 9, 10, 11, 12, 13, 14, 15, or 16 maintenance doses. In some instances, methods comprise administering a maintenance dose or doses about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, about every 20 weeks, about every 21 weeks, about every 22 weeks, about every 23 weeks, about every 24 weeks, or about every 6 months. In certain embodiments, methods comprise administering a first maintenance dose and administering a second maintenance dose about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, or about 6 months after administering the first maintenance dose.

In certain embodiments, methods comprise administering a first maintenance dose or doses about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, or about 24 weeks after administering the last loading dose.

### VI. Potency and Efficacy

In certain embodiments, described herein are methods of reducing CFB RNA and/or CFB protein in a cell or biological fluid of a human subject, wherein the methods comprise administering a therapeutically effective amount of Compound 696844 to the subject. In certain embodiments, methods reduce CFB RNA and/or CFB protein in the serum or plasma of the human subject. One may determine whether or not methods reduce CFB RNA and/or CFB protein, e.g., by detecting/quantifying a first amount of CFB RNA or CFB protein in a first biological sample obtained before administering and detecting/quantifying a second amount of CFB RNA or CFB protein in a second biological sample obtained after administering, and detecting or quantifying a reduction in CFB RNA or CFB protein by comparing the first amount to the second amount.

In certain embodiments, methods comprise reducing CFB RNA and/or CFB protein by 1-100%, or a range defined by any two of these values. In certain embodiments, methods comprise reducing CFB RNA and/or CFB protein by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In certain embodiments, methods comprise reducing CFB RNA or CFB protein by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

In certain embodiments, methods comprise reducing CFB RNA or CFB protein by about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to 100%.

In certain embodiments, methods comprise administering Compound 696844 to a subject and detecting or quantifying an amount of CFB RNA or CFB protein in a cell or a biological fluid of the subject. In certain embodiments, methods comprise detecting/quantifying a first amount of CFB RNA or CFB protein in a first biological sample obtained before administering and detecting/quantifying a second amount of CFB RNA or CFB protein in a second biological sample obtained after administering, and detecting or quantifying a reduction in CFB RNA or CFB protein by comparing the first amount to the second amount. In certain embodiments, the second biological sample is obtained less than about 24 hours after administering. In certain embodiments, the second biological sample is obtained less than about 1 week after administering. In certain embodiments, the second biological sample is obtained about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, or about 18 weeks after administering. In certain embodiments, methods comprise increasing or decreasing the dose after comparing the first amount to the second amount.

In certain embodiments, methods comprise administering more frequently or less frequently after comparing the first amount to the second amount. In certain embodiments, the biological sample is plasma. In certain embodiments, the biological fluid is serum.

VII. Overview of Diseases and Disorders Associated with Dysregulation of the Alternative Complement Pathway

Certain embodiments provided herein relate to methods of treating, preventing, or ameliorating a disease or disorder associated with dysregulation of the alternative complement pathway in a subject by administration of a therapeutically effective amount of Compound 696844.

Studies have shown that the kidney and eye share developmental pathways and structural features including basement membrane collagen IV protomer composition and vascularity (Savige et al., J Am Soc Nephrol. (2011) 22 (8): 1403-15). Inherited complement regulatory protein deficiency causes predisposition to atypical hemolytic uremic syndrome and AMD (Richards A et al., Adv Immunol. (2007) 96:141-77). Additionally, chronic kidney disease has been associated with AMD (Nitsch, D. et al., Ophthalmic Epidemiol. (2009) 16 (3): 181-6; Choi, J. et al, Ophthalmic Epidemiol. (2011) 18 (6): 259-63). Dense deposit disease (DDD), a kidney disease associated with dysregulated alternative complement pathway, is characterized by acute nephritic syndrome and ocular drusen (Martin B, Smith R J H, C3 Glomerulopathy. 2007 Jul. 20 [Updated 2018 Apr. 5]. In: Adam M P, Everman D B, Mirzaa G M, et al., editors. GeneReviews® [Internet]. Seattle (WA): University of Washington, Seattle; 1993-2022; ncbi.nlm.nih.gov/books/NBK1425/Cruz and Smith, GeneReviews (2007) July 20). Moreover, mice harboring genetic deletion of a component of the alternative complement pathway have coexisting renal and ocular disease phenotypes. It has been reported that CFH homozygous null mice develop DDD and present retinal abnormalities and visual dysfunction (Pickering et al., Nat Genet. (2002) 31 (4): 424-8). Mouse models of renal diseases associated with dysregulation of the alternative complement pathway are also accepted as models of AMD (Pennesi M E et al., Mol Aspects Med (2012) 33:487-509). CFH null mice, for example, are an accepted model for renal diseases, such as DDD, and AMD. Furthermore, it has been reported that AMD is associated with the systemic source of complement factors, which accumulate locally in the eye to drive alternative pathway complement activation (Loyet et al., Invest Ophthalmol Vis Sci. (2012) 53 (10): 6628-37).

Certain embodiments provided herein relate to methods of treating, preventing, or ameliorating an ocular disease or disorder associated with dysregulation of the alternative complement pathway in a subject by administration of a CFB specific inhibitor, such as an antisense compound targeted to CFB. In certain aspects, the ocular disease or disorder is macular degeneration, age-associated macular degeneration (AMD), including wet AMD, intermediate AMD, and dry AMD, including Geographic Atrophy; neuromyelitis optica; corneal disease, such as corneal inflammation; autoimmune uveitis; or diabetic retinopathy; or combinations thereof. In certain embodiments, the antisense compound is Compound 696844.

Certain embodiments provided herein relate to methods of treating, preventing, or ameliorating a renal disease or disorder associated with dysregulation of the alternative complement pathway in a subject by administration of a CFB specific inhibitor, such as an antisense compound targeted to CFB. In certain aspects, the renal disease or disorder is C3 glomerulopathy; atypical hemolytic uremic syndrome (aHUS); dense deposit disease (DDD, also known as MPGN Type II or C3Neph), CFHR5 nephropathy; IgA Nephropathy (IgAN); mesangiocapillary (membranoproliferative) glomerulonephritis (MPGN); autoimmune disorders including lupus nephritis and systemic lupus erythematosus (SLE); dense deposit disease (DDD); infection-induced glomerulonephritis (also known as Postinfectious glomerulonephritis); C3 glomerulonephritis (C3GN); CFHR5 nephropathy; atypical hemolytic uremic syndrome (aHUS); renal ischemia-reperfusion injury, for example, post-transplant renal ischemia-reperfusion injury, or combinations thereof. In certain embodiments, the antisense compound is Compound 696844.

Certain embodiments provided herein relate to methods of treating, preventing, or ameliorating a disease or disorder associated with dysregulation of the alternative complement pathway in a subject by administration of a CFB specific inhibitor, such as an antisense compound targeted to CFB. In certain aspects, the disease or disorder is ANCA-associated vasculitis, antiphospholipid syndrome (also known as antiphospholipid antibody syndrome (APS)), asthma, rheumatoid arthritis, Myasthenia Gravis, multiple sclerosis, preeclampsia or a metabolic disorder (such as nonalcoholic steatohepatitis (NASH)). In certain embodiments, the antisense compound is Compound 696844.

### Age-Related Macular Degeneration

Age-related macular degeneration (AMD) is a progressive disease of the macula and is the leading cause of central vision impairment in persons over the age of 50 years in developed countries (Ratnapriya and Chew 2013, Clinical Genet. 2:160-166). The disease is characterized by progressive loss of central vision, the distortion of images and straight lines, and the presence of blurry and dark areas in the central vision (Cascella et al. 2014, Ophthalmol. 582842). AMD is associated with alternative complement pathway dysregulation. Complement components are common constituents of ocular drusen, the extracellular material that accumulates in the macula of AMD patients. Furthermore, it has been reported that CFH and CFB variants account for nearly 75% of AMD cases in northern Europe and North America. It has also been found that a specific CFB polymorphism confers protection against AMD (Patel, N. et al., Eye (2008) 22 (6): 768-76). Additionally, CFB homozygous null mice have lower complement pathway activity, exhibit smaller ocular lesions, and choroidal neovascularization (CNV) after laser photocoagulation (Rohrer, B. et al., Invest Ophthalmol Vis Sci. (2009) 50 (7): 3056-64). Administration of siRNA targeting CFB protects mice from laser-induced CNV (Bora, N S et al., J Immunol. (2006) 177 (3): 1872-8). Administration of single stranded antisense oligonucleotides reduces CFB levels in the eye and plasma of C57BL/6 mice, reduces CFB RNA and plasma C3 levels in CFH^{+/-} mice, and reduces plasma levels of CFB and CFB RNA in cynomolgus monkeys (WO 2015/168635).

The underlying pathophysiology drivers for AMD are complex and the symptoms manifest in multiple related, but distinct forms. In the early and intermediate stages of AMD, the disease is characterized by the deposition of drusen, protein and lipid rich extracellular deposits between the retinal pigment epithelial (RPE) cells and Bruch's membrane (Bird et al. 1995, Ophthalmol 39:367-374; van Lookeren Campagne et al. 2014, J Pathol 232:151-164). As part of the natural course of the disease, there is a development of atrophic areas, which enlarge continuously and correspond with an absolute scotoma/geographic atrophy (Lindblad et al. 2009, Arch Ophthalmol 127:1168-1174; Grunwald et al. 2017, Ophthalmology 124:97-104). This area of geographic atrophy associated with late stage AMD corresponds with retinal regions of impaired visual function resulting from the atrophy of photoreceptor and RPE cells (Klein et al. 1991, 98:1128-1134; Sunness et al. 2007, Ophthalmology 114:271-277; Schmitz-Valckenberg et al. 2016, Ophthalmology 123:361-368).

The complement system, an important component of innate immunity, is the most widely accepted pathogenic pathway of the immune system implicated in AMD, and genome wide association studies (GWAS) and rare variant analyses suggest the alternative complement pathway (AP) is overactive in AMD (Kijlstra et al. 2005, Ocul Immunol Inflamm 13:3-11; Donoso et al., 2006 Surv Ophthalmol 51:137-152; Anderson et al. 2010, Prog Retin Eye Res 29:95-112; Whitmore et al. 2015, Prog Retin Eye Res 45:1-29; Cao et al. 2016, Br J Ophthalmol 100:713-718; Geerlings et al. 2017, Mol Immunol 84:65-76). Specific polymorphisms of complement factor H (CFH), which is the negative regulator of the AP, confer increased risk for AMD (Donoso et al. 2010, Surv Ophthalmol 55:227-246; Heurich et al. 2011, Proc Natl Acad Sci USA 108:8761-8766). In contrast, specific polymorphisms of Factor B, the positive regulator, confer protection against AMD (Gold et al. 2006 Nat Genet 38:458-462; Montes et al. 2009; Proc Natl Acad Sci USA 106:4366-4371; Heurich et al. 2011; Mantel et al. 2014, Ophthalmic Genetics 35:12-17).

CFB is synthesized primarily by the liver (Koskimies et al., Complement Inflamm 1991; 8:257-260; Morgan and Gasque Clin Exp Immunol 1997; 107:1-7; Marsh et al. Atherosclerosis 2002; 162:227-244) and at very low levels in several extrahepatic sites (Whaley J Exp Med 1980; 151:501-516; Ripoche et al. J Exp Med 1988; 168:1917-1922; Strunk et al. J Clin Invest 1988; 81:1419-1426; Matsumoto et al. Proc Natl Acad Sci USA 1997; 94:8720-8725). Ocular CFB is located predominately in the choroidal capillaries and Bruch's membrane region and not evident in the neural retina (Loyet et al. Invest Ophthalmol Vis Sci 2012; 53:6628-6637). Hence, choroidal CFB appears to be derived from systemic sources. Plasma concentrations of complement alternative pathway activation products were found to be significantly elevated in AMD patients compared to controls (Scholl et al. PLOS One 2008; 3: e2593; Reynolds et al. Invest Ophthalmol Vis Sci 2009; 50:5818-5827; Loyet et al. 2012; Paun et al. Sci Rep 2016; 6:26568; PLOS One 2016; 11: e0144367). This suggests an ongoing systemic activation of the alternative complement pathway in AMD pathogenesis and adds to the increasing evidence that AMD is a systemic disease with local disease manifestation in the ageing macula (Smailhodzic et al. 2012, Ophthalmology 119:339-346; Br J Ophthalmol 2016; 100:713-718).

### IgA Nephropathy

IgA Nephropathy (IgAN) is the most prevalent primary chronic glomerulonephritis worldwide and is an important cause of chronic kidney failure (Maillard et al. 2015, J Am Soc Nephrol 26:1503-1512). IgAN is characterized by immunodeposits with dominant or co-dominant IgA in the glomerular mesangium of the kidneys, resulting in inflammation and tissue damage (Maillard et al. 2015). Although IgAN may occur at any age, it generally presents in the second or third decade of life. The clinical presentation, disease progression and histologic findings are highly variable among affected individuals.

IgAN is characterized by microscopic and/or macroscopic hematuria sometimes in the setting of an acute illness such as a respiratory tract infection or gastroenteritis. Over time, affected individuals may develop proteinuria and hypertension. Up to 40% of affected individuals will experience a progressive decline in kidney function, developing end-stage kidney disease (ESKD) up to 20 years from the date of their diagnostic kidney biopsy (Maillard et al. 2015). Patients with ESKD require dialysis or kidney transplant and ESKD is associated with a significant risk of premature mortality and reduced quality of life comparable to that of certain cancers and heart disease (Chen et al. 2016, Blood Purif. 41 (1-3): 218-24).

IgAN is broadly categorized as primary or secondary (i.e., associated with a systemic disease) (Rizk et al. 2019, Frontiers Immunol 10:504). IgAN can manifest without extra-renal involvement, or as part of a systemic vasculitis phenotype currently referred to as IgA vasculitis with nephritis (previously Henoch Schönlein pupura nephritis). IgAN may be diagnosed with a renal biopsy. The hallmark of IgA Nephropathy is the deposition of IgA in the glomerular mesangium. Ninety percent of renal biopsies in patients with IgAN also contain C3 in the same distribution as IgA deposits (Maillard et al. 2015). Plasma C3 levels are generally within the normal range; although complement activation products may be elevated in the plasma supporting the role of the alternative pathway in IgAN. On renal biopsy, in addition to IgA deposits, there are glomerular deposits derived from overactivity of the alternative pathway (C3 and properdin), the lectin pathway (C4d), as well as the terminal product of the complement cascade (membrane attack complex C5b-9C) (Wyatt and Julian 2013, N Engl J Med 368:2402-2414). Deposition of C1q is generally not observed (Wyatt and Julian 2013). Therefore, data support that both the alternative complement and the lectin complement pathways have roles in triggering renal injury in IgAN.

CFB circulates in the blood as a proenzyme and is a key protease in the alternative pathway (AP) of Complement CFB associates with C3 in the fluid phase via the continual production or spontaneous hydrolysis of C3 or “tick-over” or associates with Complement Protein C3b (C3b) (activated form of C3) attached to the target; its cleavage by Factor D into Ba and Bb leads to activation of CFB (Maillard et al. 2015). This results in formation of C3Bb or amplification of C3 convertase (C3bBb) which then triggers the amplification loop of the AP. As a result, C3b molecules attach to the target surface activating the terminal complement cascade that results in the formation of C5b-9 (Maillard et al. 2015; Thurman 2017, Nephrol Dial Transplant 32:157-i64). The C5b-9 membrane attack complex creates pores in the cell membrane with resultant influx of calcium, cell activation and glomerular injury. Of note, deposits of the membrane attack complex have been observed in renal biopsies of individuals with IgAN (Maillard et al. 2015). Upon reducing CFB, the formation of Complement Protein C3a (C3a), Complement Factor C5a (C5a) and the membrane attack complex are potentially reduced and the consequent inflammatory damage to the kidney is potentially attenuated.

In certain embodiments, provided herein are methods for ameliorating IgA Nephropathy and/or delaying the progression of renal failure associated with primary IgA Nephropathy by systemic administration of Compound 696844. Compound 696844 targets CFB messenger ribonucleic acid (mRNA) in the liver, and reduces levels of plasma FB. The reduction of plasma CFB, an essential component of the AP, diminishes the hyper-activation of the AP that contributes to primary IgA Nephropathy pathology. As demonstrated in Example 2, reducing the level of plasma CFB in subjects with IgAN, diminished the hyper-activation of the AP, with a resulting reduction in proteinuria. Administration of single stranded antisense oligonucleotides reduces CFB levels in the plasma of C57BL/6 mice, reduces CFB RNA and plasma C3 levels in CFH^{+/-} mice, reduces renal C3 accumulation in a mouse lupus model, and reduces plasma levels of CFB and CFB RNA in cynomolgus monkeys (WO 2015/168635).

### VIII. Assessing Efficacy of Compound 696844

### Reduction of Plasma CFB

In certain embodiments, methods described herein are sufficiently effective to reduce the level of plasma CFB in a subject. In certain embodiments, methods described herein are sufficiently effective to reduce the level of plasma CFB in a subject as assessed by percent reduction of plasma CFB between one time point and a later time point following administration of Compound 696844. In some embodiments, the percent reduction in the level of plasma CFB is assessed by comparing the level of plasma CFB measured before the first administration of Compound 696844 (baseline) to the level of plasma CFB measured at a time point after administration of one or more doses of Compound 696844. In some embodiments the percent reduction is calculated as the percent reduction of the level of plasma CFB 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first administration of Compound 696844 from the baseline 24-hour protein excretion. In some embodiments the percent reduction is calculated as the percent reduction of the level of plasma CFB between one time point after administration of one or more doses of Compound 696844 and a second time point 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first time point. In certain embodiments, the level of CFB may be determined by a radial immunodiffusion assay, an ELISA assay, or by nephelometry.

### Reduction of Serum AH50 Activity

In certain embodiments, methods described herein are sufficiently effective to reduce serum AH50 activity in a subject. In certain embodiments, methods described herein are sufficiently effective to reduce serum AH50 activity in a subject as assessed by percent reduction of serum AH50 activity between one time point and a later time point following administration of Compound 696844. In some embodiments, the percent reduction in serum AH50 activity is assessed by comparing the level of serum AH50 activity measured before the first administration of Compound 696844 (baseline) to the level of serum AH50 activity measured at a time point after administration of one or more doses of Compound 696844. In some embodiments the percent reduction is calculated as the percent reduction of serum AH50 activity 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first administration of Compound 696844 from the baseline serum AH50 activity. In some embodiments the percent reduction is calculated as the percent reduction of serum AH50 activity between one time point after administration of one or more doses of Compound 696844 and a second time point 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first time point. In certain embodiments, AH50 activity may be determined by use of a hemolytic assay (AH50 Hemolytic). In some embodiments, AH50 activity may be determined by use of a WIESLAB Complement System Alternative Pathway assay (SVAR LIFE SCIENCE) (AH50 WAP).

### Reduction in Urine Protein Excretion

In certain embodiments, methods described herein are sufficiently effective to reduce urine protein excretion in a subject (proteinuria). In certain embodiments, methods described herein are sufficiently effective to reduce urine protein excretion in a subject as assessed by percent reduction in 24-hour urine protein excretion between one time point and a later time point following administration of Compound 696844. In some embodiments, the percent reduction in 24-hour protein excretion is assessed by comparing the 24-hour protein excretion measured before the first administration of Compound 696844 (baseline) to the 24-hour urine protein excretion measured at a time point after administration of one or more doses of Compound 696844. In some embodiments the percent reduction is calculated as the percent reduction of 24-hour protein excretion 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first administration of Compound 696844 from the baseline 24-hour protein excretion. In some embodiments the percent reduction is calculated as the percent reduction of 24-hour protein excretion between one time point after administration of one or more doses of Compound 696844 and a second time point 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first time point.

In certain embodiments, methods described herein are sufficient to reduce urine protein excretion in a subject as assessed by absolute reduction in urine protein excretion between baseline and a time point 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after administration of one or more doses of Compound 696844. In certain embodiments, methods described herein are sufficient to reduce albumin excretion in a subject as assessed by absolute reduction in albuminuria (“urine albumin/creatinine” or “UAC ratio”) between baseline and a time point 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after administration of one or more doses of Compound 696844. In certain embodiments, methods described herein are sufficient to reduce protein excretion in a subject as assessed by absolute reduction in proteinuria (UPC ratio) between baseline and a time point 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after administration of one or more doses of Compound 696844.

In certain embodiments, methods described herein are sufficient to reduce urine protein excretion in a subject as assessed by absolute reduction in urine protein excretion between one time point after administration of one or more doses of Compound 696844 and a second time point 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first time point. In certain embodiments, methods described herein are sufficient to reduce albumin excretion in a subject as assessed by absolute reduction in albuminuria (UAC ratio) between one time point after administration of one or more doses of Compound 696844 and a second time point 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first time point.

In some embodiments, urine protein excretion may be measured in a single sample (spot sample), for example a first voided, or first morning sample, or in an aliquot of a collection of multiple samples, for example from a 24 hour collection. Urine protein excretion may be measured as urine protein ((grams/day) 2 (g/day) 2), and by urine protein to creatinine ratio (UPCr) 2.

### Reduction in Serum CH50 Activity

In certain embodiments, methods described herein are sufficiently effective to reduce serum CH50 activity in a subject. In certain embodiments, methods described herein are sufficiently effective to reduce serum CH50 activity in a subject as assessed by percent reduction or absolute reduction of serum CH50 activity between one time point and a later time point following administration of Compound 696844. In some embodiments, the percent reduction in serum CH50 activity is assessed by comparing the level of serum CH50 activity measured before the first administration of Compound 696844 (baseline) to the level of serum CH50 activity measured at a time point after administration of one or more doses of Compound 696844. In some embodiments the percent reduction is calculated as the percent reduction or absolute reduction of serum CH50 activity 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first administration of Compound 696844 from the baseline of serum CH50 activity. In some embodiments the percent reduction or absolute reduction is calculated as the percent reduction or absolute reduction of serum CH50 activity between one time point after administration of one or more doses of Compound 696844 and a second time point 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first time point. In some embodiments, the level of CH50 activity may be determined by a hemolytic assay.

Reduction in Urine Ba and sC5b-9

In certain embodiments, methods described herein are sufficiently effective to reduce the level of urine Ba or sC5b-9 in a subject. In certain embodiments, methods described herein are sufficiently effective to reduce the level of urine Ba or sC5b-9 in a subject as assessed by percent reduction or absolute reduction of the level of urine Ba or sC5b-9 between one time point and a later time point following administration of Compound 696844. In some embodiments, the percent reduction in the level of urine Ba or sC5b-9 is assessed by comparing the level of urine Ba or sC5b-9 measured before the first administration of Compound 696844 (baseline) to the level of urine Ba or sC5b-9 measured at a time point after administration of one or more doses of Compound 696844. In some embodiments the percent reduction is calculated as the percent reduction or absolute reduction of the level of urine Ba or sC5b-9 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first administration of Compound 696844 from the baseline level of urine Ba or sC5b-9. In some embodiments the percent reduction or absolute reduction is calculated as the percent reduction or absolute reduction of the level of urine Ba or sC5b-9 between one time point after administration of one or more doses of Compound 696844 and a second time point 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first time point. In certain embodiments, the level of Bb may be determined by use of an assay such as QUIDEL MICROVUE sC5b-9 PLUS EIA.

### Reduction of Bb

In certain embodiments, methods described herein are sufficiently effective to reduce the level of Bb in serum, plasma, or urine in a subject. In certain embodiments, methods described herein are sufficiently effective to reduce the level of Bb in a subject as assessed by percent reduction of Bb between one time point and a later time point following administration of Compound 696844. In some embodiments, the percent reduction in the level of Bb is assessed by comparing the level of Bb measured before the first administration of Compound 696844 (baseline) to the level of Bb measured at a time point after administration of one or more doses of Compound 696844. In some embodiments the percent reduction is calculated as the percent reduction of the level of Bb 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first administration of Compound 696844 from the baseline 24-hour protein excretion. In some embodiments the percent reduction is calculated as the percent reduction of the level of Bb between one time point after administration of one or more doses of Compound 696844 and a second time point 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first time point. In certain embodiments, the level of Bb may be determined by use of an assay such as QUIDEL MICROVUE Bb PLUS EIA.

### Slowing of Progression of Geographic Atrophy Area

In certain embodiments, methods described herein are sufficiently effective to slow the progression of the Geographic Atrophy Area in a subject. In certain embodiments, administration of Compound 696844 delays the progression of the total Geographic Atrophy area in an eye, in certain embodiments, administration of Compound 696844 delays the appearance of additional areas of Geographic Atrophy in an eye. In certain embodiments, methods described herein are sufficiently effective to slow the progression of the Geographic Atrophy Area in a subject as assessed by percent reduction of the rate of change of the area of GA measured by fundus autofluorescence (FAF) or by spectral-domain optical coherence tomography (SD-OCT) between one time point and a later time point following administration of Compound 696844. In some embodiments, the rate of change of the area of GA is assessed by comparing the area of GA measured before the first administration of Compound 696844 (baseline) to the area of GA measured at a time point after administration of one or more doses of Compound 696844. In some embodiments, the rate of change of the area of GA is assessed by comparing rate of change of the area of GA calculated from two or more measurements at two or more time points before the first administration of Compound 696844 (baseline) to the rate of change of the area of GA calculated from two or more measurements at two or more time points after administration of one or more doses of Compound 696844. In some embodiments the percent reduction is calculated as the percent reduction of area or reduction of the rate of change of the area of GA 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first administration of Compound 696844 from the baseline of the area of GA or the rate of change of the area of GA.

### Slowing of Reduction of Low Luminance Visual Acuity (LLVA).

In certain embodiments, methods described herein are sufficiently effective to slow the reduction of LLVA in a subject. In certain embodiments, administration of Compound 696844 delays the reduction of LLVA in an eye. In certain embodiments, methods described herein are sufficiently effective to slow the reduction of LLVA in a subject as assessed by percent reduction of LLVA Score measured between one time point and a later time point following administration of Compound 696844. In some embodiments, the rate of change of LLVA Score is assessed by comparing LLVA measured before the first administration of Compound 696844 (baseline) to LLVA measured at a time point after administration of one or more doses of Compound 696844. In some embodiments, the rate of change of LLVA is assessed by comparing rate of change of LLVA Score calculated from two or more measurements at two or more time points before the first administration of Compound 696844 (baseline) to the rate of change of LLVA Score calculated from two or more measurements at two or more time points after administration of one or more doses of Compound 696844. In some embodiments the percent reduction is calculated as the percent reduction of LLVA Score or reduction of the rate of change of LLVA Score 1, 2, 3, 4, 5, 6, 7, 8, 9, 11, 12, 13, 14, 15, 16 17, 18, 19, 20, 21, 22, 23, 24, 25, 26, 27, 28, 29, or 30 weeks after the first administration of Compound 696844 from the baseline of LLVA or the rate of change of LLVA Score.

### Slowing of Morphological Changes in Choriocapillaris

In certain embodiments, methods described herein are sufficiently effective to slow the progression of morphological changes in choriocapillaris in a subject. In certain embodiments, administration of Compound 696844 delays the progression of morphological changes in choriocapillaris in an eye. In certain embodiments, methods described herein are sufficiently effective to slow the progression of morphological changes in choriocapillaris in a subject as assessed by percent of morphological changes in choriocapillaris measured by measured by optical coherence tomography angiography (OCTA) between one time point and a later time point following administration of Compound 696844. In some embodiments, the rate of change of morphological changes in choriocapillaris is assessed by comparing morphological changes in choriocapillaris measured before the first administration of Compound 696844 (baseline) to morphological changes in choriocapillaris measured at a time point after administration of one or more doses of Compound 696844. In some embodiments, the rate of change of morphological changes in choriocapillaris is assessed by comparing rate of change of morphological changes in choriocapillaris calculated from two or more measurements at two or more time points before the first administration of Compound 696844 (baseline) to the rate of change of morphological changes in choriocapillaris calculated from two or more measurements at two or more time points after administration of one or more doses of Compound 696844. In some embodiments the percent reduction is calculated as the percent reduction of morphological changes in choriocapillaris or reduction of the rate of morphological changes in choriocapillaris 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first administration of Compound 696844 from the baseline of morphological changes in choriocapillaris or the rate of morphological changes in choriocapillaris.

### Slowing of Reduction of Best Corrected Visual Acuity (BCVA)

In certain embodiments, methods described herein are sufficiently effective to slow the reduction of BCVA in a subject. In certain embodiments, administration of Compound 696844 delays the reduction of BCVA in an eye. In certain embodiments, methods described herein are sufficiently effective to slow the reduction of BCVA in a subject as assessed by eye chart or computer, between one time point and a later time point following administration of Compound 696844. In some embodiments, the rate of change of BCVA is assessed by comparing BCVA measured before the first administration of Compound 696844 (baseline) to BCVA measured at a time point after administration of one or more doses of Compound 696844. In some embodiments, the rate of change of BCVA is assessed by comparing rate of change of BCVA calculated from two or more measurements at two or more time points before the first administration of Compound 696844 (baseline) to the rate of change of BCVA calculated from two or more measurements at two or more time points after administration of one or more doses of Compound 696844. In some embodiments the percent reduction is calculated as the percent reduction of BCVA or reduction of the rate of change of BCVA 1, 2, 3, 4, 5, 6, 7, 8, 9, 10 11, 12, 13, 14, 15, 16 17, 18, 19, 20, 21, 22, 23, 24, 25, 26, 27, 28, 29, or 30 weeks after the first administration of Compound 696844 from the baseline of BCVA or the rate of change of BCVA.

In certain embodiments, methods described herein are sufficiently effective to ameliorate at least one symptom or hallmark of a disease or disorder associated with CFB protein in a human subject. In certain embodiments, methods described herein are sufficiently effective to prevent or decrease the rate of progression, or delay the onset of at least one symptom or hallmark associated with CFB protein in a human subject. In certain embodiments, the disease or disorder is an ocular disease or disorder or a kidney disease or disorder. In certain embodiments, the disease or disorder is age-related macular degeneration (AMD), wet AMD, intermediate AMD, dry AMD, Geographic Atrophy associated with AMD (GA), IgA Nephropathy (IgAN), systemic lupus erythematosus (SLE), dense deposit disease (DDD), C3 glomerulonephritis (C3GN), CFHR5 nephropathy, atypical hemolytic uremic syndrome (aHUS), aHUS characterized by thrombotic microangiopathy, ANCA-associated vasculitis, antiphospholipid syndrome (also known as antiphospholipid antibody syndrome (APS)), asthma, rheumatoid arthritis, Myasthenia Gravis, multiple sclerosis, preeclampsia or a metabolic disorder (such as NASH). In certain embodiments, the at least one symptom or hallmark is proteinuria, reduction in kidney function, C3 deposits in kidney, hematuria, hypertension, inflammation in kidney, IgA deposits in glomerular mesangium, hyper-active alternative complement pathway, elevated plasma CFB, elevated serum, plasma, or urine Bb, elevated serum, plasma, or urine Ba, elevated serum, plasma or urine sC5b-9, elevated serum, plasma, or urine C3, elevated serum, plasma, or urine C4, elevated serum AH50 activity, reduction in visual acuity, reduction in low luminescence visual acuity, reduction in best corrected visual acuity, central vision loss, area of geographic atrophy, number of areas of geographic atrophy, or morphological changes in choriocapillaris.

### IX. Certain Combination Therapies

In certain embodiments, methods comprise co-administering Compound 696844 with at least one other pharmaceutical agent. In certain embodiments, the at least one other pharmaceutical agent ameliorates a disease or disorder associated with dysregulation of the complement pathway. In certain embodiments, the at least one other pharmaceutical agent ameliorates a disease or disorder associated with dysregulation of the alternative complement pathway. In certain embodiments, the at least one other pharmaceutical agent ameliorates a disease or disorder associated with CFB protein. In certain embodiments, the at least one other pharmaceutical agent ameliorates a symptom or hallmark of a disease or disorder associated with an overactive alternative complement pathway. In certain embodiments, Compound 696844 is co-administered with the at least one other pharmaceutical agent to produce a combinational effect. In certain embodiments, Compound 696844 is co-administered with the at least one other pharmaceutical agent to produce a synergistic effect.

In certain embodiments, Compound 696844 and the at least one other pharmaceutical agent are administered at the same time. In certain embodiments, Compound 696844 and the at least one other pharmaceutical agent are administered at different times. In certain embodiments, Compound 696844 and the at least one other pharmaceutical agent are prepared together in a single formulation. In certain embodiments, Compound 696844 and the at least one other pharmaceutical agent are administered are prepared in separate formulations.

In certain embodiments, pharmaceutical agents that may be co-administered with Compound 696844 include agents such as angiotensin converting enzyme inhibitors (ACEi), angiotensin receptor blockers (ARB), or sodium/glucose cotransporter-2 inhibitors (SGLT2i), or agents directed to renal diseases or disorders; anti-complement agents or complement inhibitors; agents directed to AMD, or other agents directed to ocular diseases or disorders.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Phase 1 Human Clinical Trial with Compound 696844

Compound 696844 was formulated for administration as a sterile, parenteral solution of 100 mg/mL of the compound in phosphate buffered saline, adjusted to approximately pH 7.4 with acid or base during compounding. The solution was clear and colorless to light yellow in color. The medicinal product was packaged as a 0.8 mL deliverable volume in an ISO 2R Type I, clear glass vial that was stoppered with a butyl rubber closure and sealed with an aluminum overseal. Approximately 1.0 mL was filled into each vial to ensure the labeled 0.8 mL volume was available for dosing.

Compound 696844 was evaluated in two phase 1 double-masked, placebo-controlled, dose escalation studies of single or multiple doses of Compound 696844 administered subcutaneously by injection to healthy adult human subjects.

A total of 54 healthy human subjects, age range 27-65 years, were randomized to receive placebo (phosphate buffered saline (PBS)) or Compound 696844 in PBS. In the single ascending dose study, eighteen (18) subjects were administered 10, 20, or 40 mg of Compound 696844, and 6 were administered placebo, by subcutaneous injection, and were followed during post-treatment period for 90 days.

In the multiple ascending dose study, 12 subjects were administered 10 mg of Compound 696844, 12 subjects were administered 20 mg of Compound 696844, and 6 subjects were administered placebo, by subcutaneous injection. Each subject was administered 8 doses in 36 days, 4 loading doses in the first two weeks (Days 1, 4, 8, and 12), then once-a-week for the next 4 weeks (Days 15, 22, 29, and 36).

TABLE 1
Phase 1 Study in Healthy Volunteers

| | Single Dose | Multiple Doses |
|---|---|---|
| Number (N) | 24 (6/cohort) | 30 (6/placebo; |
| | | 12 drug dose) |
| Gender | 21/3 | 22/8 |
| (male/female) | | |
| Ages (ranges) | 21-56 | 25-65 |
| Race | 24/0/0 | 27/2/1 |
| (white/Asian/Maori) | | |

Ocular and systemic safety, pharmacokinetic (PK) activity, and pharmacodynamic activity (CFB and ACP activity (AH50)) were assessed in both single and multiple ascending dose studies.

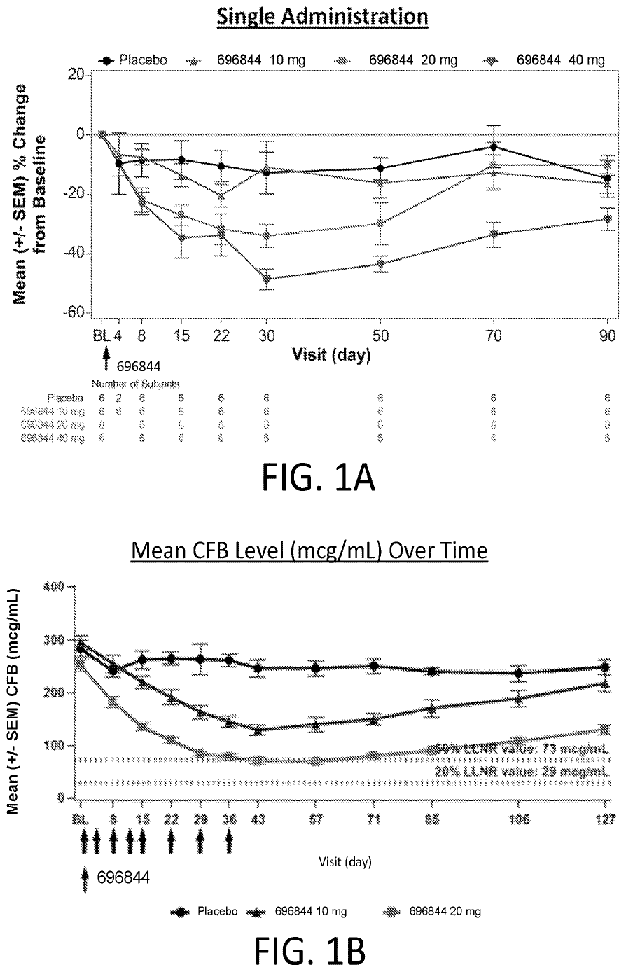

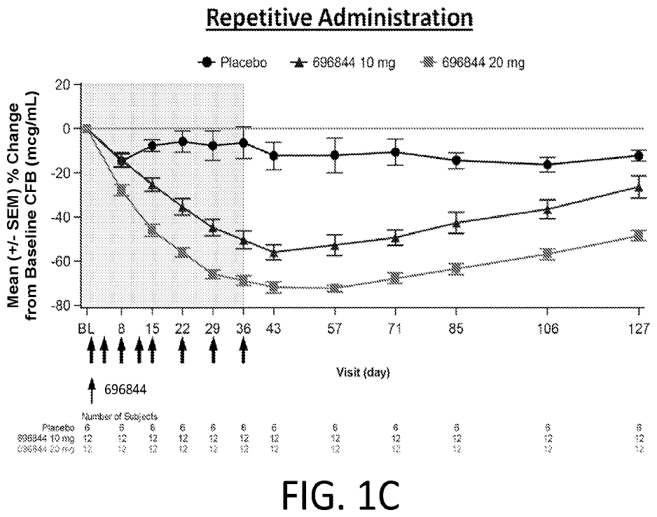

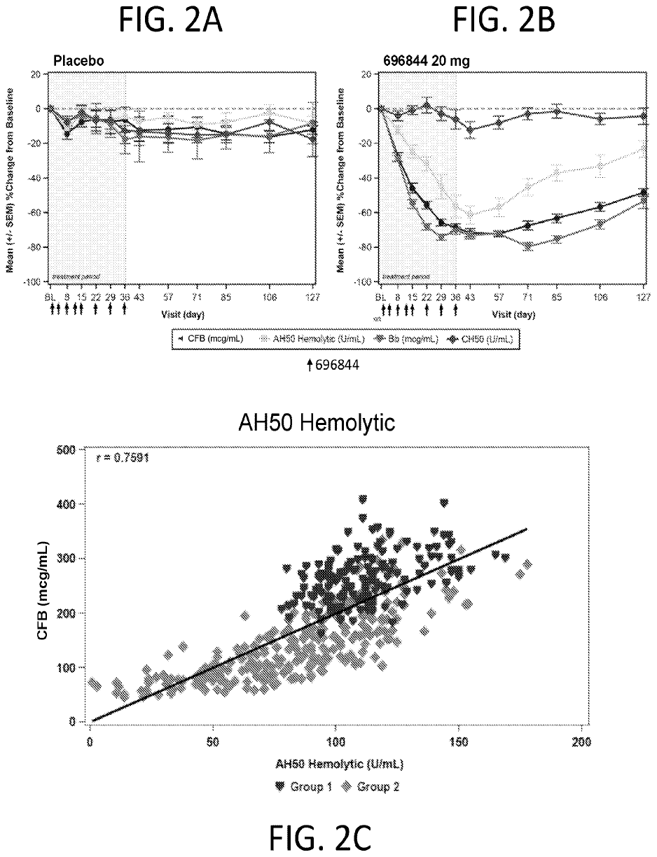

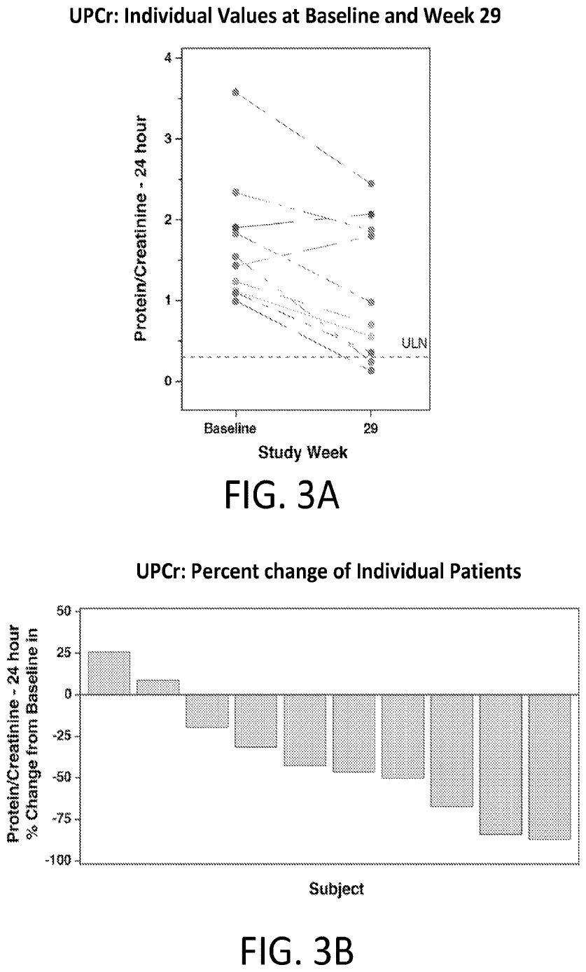

All subjects completed the Phase 1 study (week 19). There were no safety signals or clinically-relevant changes in ocular endpoints (BCVA, IOP, comprehensive examination), blood chemistries, hematology or vital signs following Compound 696844 dosing. Administration of single and multiple doses of Compound 696844 to healthy volunteers resulted in statistically significant, dose-dependent reductions in plasma CFB levels compared to placebo, with the nadir following multiple doses occurring on Day 43 (56% reduction with 10 mg and 72% reduction with 20 mg) (FIG. 1B, FIG. 2). Serum Factor B functional (Factor BF) activity and Factor B split product were similarly reduced.

Complement assays used include the following: CFB level (radial immunodiffusion); Factor B function, AH50 activity, and CH50 activity (hemolytic assay); Bb level (QUIDEL MICROVUE Bb PLUS EIA; ELISA immunoassay); AH50 activity WAP (Weislab ELISA); sC5b-9 levels (MICROVUE sC5b-9 PLUS Enzyme immunoassay); C5a levels (QUIDEL C5a ELISA); C2 (radial immunodiffusion); C3 (SPAplus (Binding Site)); C3a (QUIDEL C3a PLUS EIA; ELISA immunoassay); C4 (SPAplus (Binding Site)); Factor D levels (R & D SYSTEMS QUANTIKINE kit; ELISA immunoassay).

Single administrations of Compound 696844 resulted in a dose-dependent reduction of plasma CFB levels, shown as percentage change from Baseline over time by treatment group (FIG. 1A). Results for placebo-treated subjects varied over time but remained with 2 SD of normal. The nadir for CFB reduction was generally reached between 15 and 30 days after dosing. Averaged over Days 15, 22, and 30, the mean percent change from Baseline with Compound 696844 was-15% with 10 mg, −31% with 20 mg, and −39% with 40 mg compared to −11% with placebo. The difference versus placebo was statistically significant for 40 mg (p=0.030) and approached statistical significance with 20 mg (p=0.054).

Following single administration of Compound 696844 the serum CFB functional levels (reported as Factor B function) and Factor B split product (Bb) were similarly reduced with Compound 696844 treatment. However, the reductions of Factor B function were not of sufficient magnitude to cause consistent changes in serum alternative pathway activity (AH50). In addition, there were no consistent changes in either serum classical complement activity, as measured by CH50 and sC5b-9, or compensatory changes in Factor D or C3 levels.

Multiple administrations of Compound 696844 to healthy subjects resulted in a dose-dependent reduction of plasma CFB, shown as actual values in FIG. 1B and as percentage change from Baseline over time by treatment group in FIG. 1C. Results for placebo-treated subjects varied over time but remained with 2 SD of normal. The nadir for CFB reduction was reached at Day 43 (56% reduction with 10 mg and 72% with 20 mg). Averaged over Days 29, 36, 43, and 57, the mean percent change from Baseline with Compound 696844 was 51% with 10 mg and 70% with 20 mg compared to 10% with placebo.

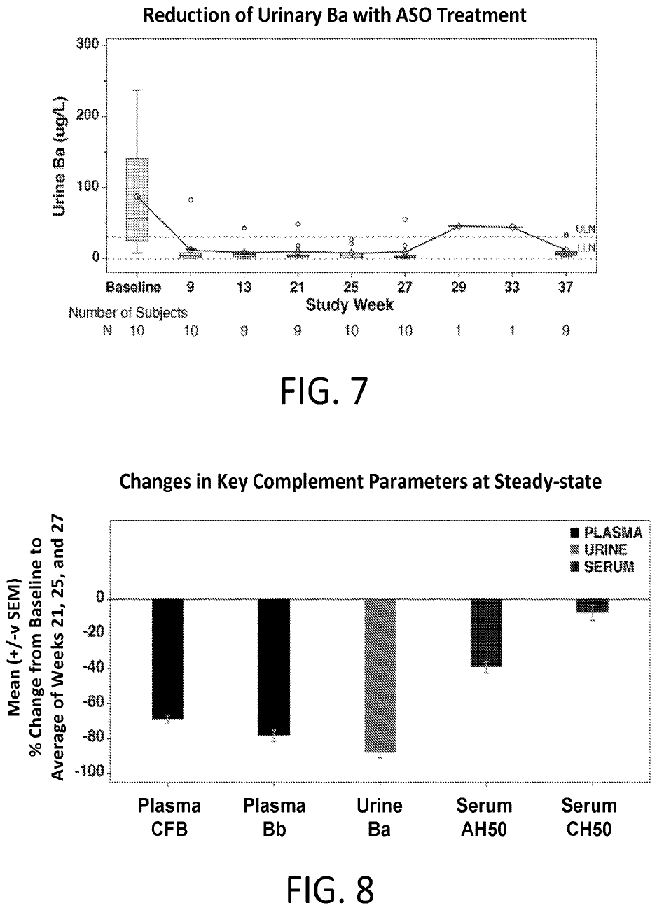

The greater reductions of CFB levels following multiple administrations resulted in greater reductions of serum Factor B function levels and Bb levels (FIG. 6 and FIG. 7).

Multiple administrations of Compound 696844 resulted in a reduction of plasma CFB levels and serum Bb levels (FIG. 2A and FIG. 2B). A modest (˜25%) reduction of AH50 hemolytic activity was observed with multiple doses of 10 mg SC and a greater reduction (˜55%) with the 20 mg dose. AH50 hemolytic activity correlated with plasma CFB levels (FIG. 2C). Even with the substantial reduction of CFB, the maximum reduction in CH50 with Compound 696844 treatment was only 11% with 10 mg (Day 85) and 12% with 20 mg (Day 43). No clinically relevant changes were observed in other complement components, including Factor D, C2, C3 and C4. The mean (+/−SE) % reductions of AH50, and classical component pathway activity (CH50) levels for the 20 mg dose on day 43 (7 days post last administration) was −62 (+/−5) and −12 (+/−5) % (FIG. 2B).

There were no meaningful safety or tolerability findings after 2 months of exposure to Compound 696844, with no serious adverse events, and mild to moderate adverse events with a comparable incidence with the placebo treatment group. No increased incidence of infection was found. No injection site reactions, or clinically relevant changes in hematology, serum chemistry, liver function, or renal function were recorded.

### Example 2: Phase 2a Human Clinical Trial with Compound 696844 for Treatment of Kidney Disease

An exploratory, single arm, open label Ph 2 study was conducted to evaluate Compound 696844 in patients with primary IgA Nephropathy. Subjects had biopsy-confirmed IgA Nephropathy (IgAN) diagnosed within 12 months, demonstrating≤50% interstitial fibroses, ≤50% crescents, and presence of C3 deposition; proteinuria from a 24-hour collection of between 1.5 g/day to 5.0 g/day; hematuria; an eGFR>40 mL/min/1.73 m²; and had not been treated with immunosuppressive/immunomodulatory medications within 12 months.

An interim analysis of the ongoing open label study was conducted following administration of Compound 696844 by subcutaneous injection at (70 mg/month, with two administrations two weeks apart during the first month). Subjects were administered the compound at weeks 1, 3, 5 and every 4 weeks thereafter for 20 weeks (7 administrations). The final administration was at week 25. The primary outcome was a reduction in 24-hr proteinuria at week 29 (4 weeks after last dose) compared to baseline (BL (prior to administration of Compound 696844)). Proteinuria is indicated by urine protein ((grams/day)² (g/day)²), and by urine protein to creatinine ratio (UPCr)². Secondary outcome measures included: safety, complement levels and estimated glomerular flow rate (eGFR).

Complement assays include assays listed in Example 1.

The study enrolled 10 subjects, ages 25-59 yr, 40% Female, 6 Asian, and 4 White.

TABLE 2
Phase 2 Kidney Disease Study, Individual Demographics

| | | | | | Proteinuria | Proteinuria |
|---|---|---|---|---|---|---|
| Patient | Age | Gender | Race | eGFR¹ | (g/day)² | (UPCr)² |
| A | 54 | M | White | 56 | 1.89 | 1.12 |
| B | 25 | M | Asian | 126 | 5.59 | 3.58 |
| C | 39 | F | Asian | 88 | 0.71 | 1.43 |
| D | 44 | F | Asian | 67 | 1.09 | 1.00 |
| E | 31 | F | Asian | 41 | 2.95 | 1.90 |
| F | 42 | M | Asian | 64 | 3.51 | 1.83 |
| G | 56 | F | White | 39 | 1.43 | 1.55 |
| H | 42 | M | Asian | 84 | 1.67 | 1.10 |
| I | 34 | M | White | 73 | 2.24 | 1.23 |
| J | 59 | M | White | 51 | 4.78 | 2.33 |

¹eGFR (mL/min/1.73 m²) based on CKD-EPI 2009 on Day 1
²Baseline proteinuria determined from two 24-hr collections during baseline/screening period

TABLE 3
Individual Renal Histological Analysis

| Patient | MHS | EHS | SSS | TAIFS | CS | C3 |
|---|---|---|---|---|---|---|
| A | M0 | E0 | S1 | T1 | C0 | 3+ |
| B | M1 | E0 | S1 | T1 | C1 | 3+ |
| C | M0 | E0 | S1 | T0 | C1 | 2+ |
| D | M1 | E1 | S1 | T0 | C0 | 2 |
| E | M1 | E0 | S1 | T1 | C0 | 1+ |
| F | M1 | E0 | S1 | T1 | C0 | 2 |
| G | M1 | E0 | S1 | T0 | C0 | 3+ |
| H | M0 | E0 | S1 | T0 | C1 | 1 |
| I | M1 | E0 | S1 | T1 | C0 | 2 |
| J | M1 | E0 | S1 | T1 | C0 | 2-3 |

C3: C3 deposition Score; CS: Crescent Score; EHS: Endocapillary Hypercellularity Score; MHS: Mesangial Hypercellularity Score; SSS: Segmental Sclerosis Score; TAIFS: Tubular Atrophy/Interstitial Fibrosis Score.

Compound 696844 reduced the level of plasma CFB in subjects with IgAN, diminished the hyper-activation of the AP, and reduced proteinuria in subjects diagnosed with IgAN. Plasma CFB levels of IgAN patients were measured following multiple administrations by subcutaneous injection of 70 mg of Compound 696844 on Weeks 1, 3, 5, 9, 13, 17, 21 and 25. Plasma CFB levels of IgAN patients were reduced 71% at week 9, the first time of sampling, and maintained low levels during treatment, as measured by nephelometry assay (FIG. 6). At Week 37, 12 weeks after the last administration, the plasma CFB levels were below LLN. The steady state (average of Weeks 21, 25 and 27) levels of plasma CFB were decreased 69%.

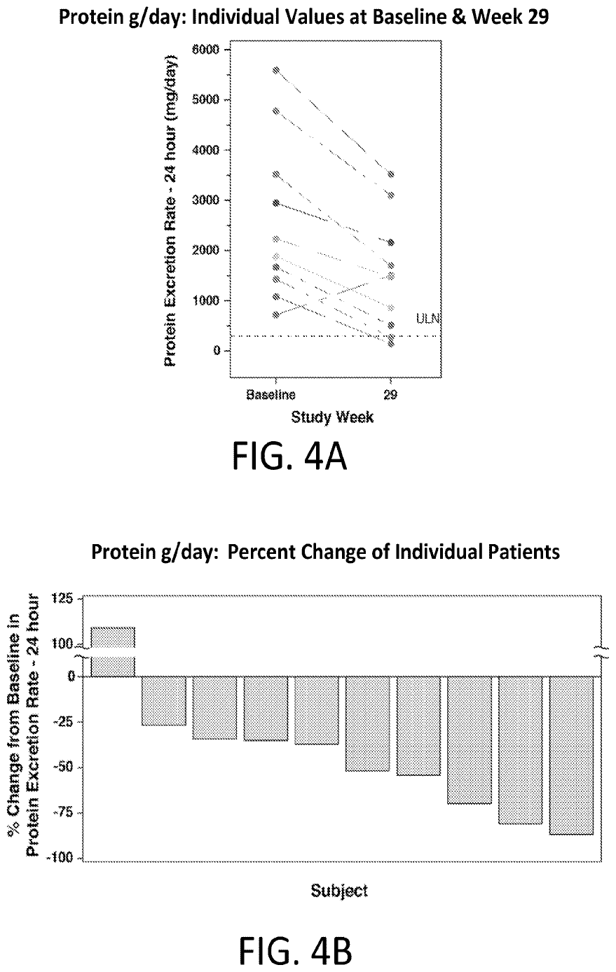

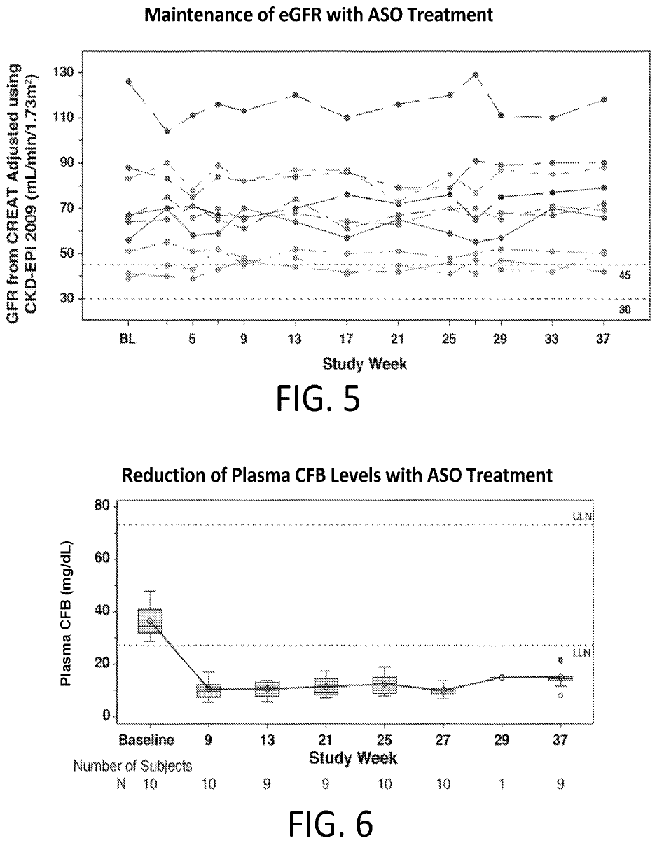

There was a selective reduction of plasma CFB protein levels, serum AP activity and urinary Ba from baseline to end of treatment (mean % change of −69%, −39% and −88%, respectively) (FIG. 8). Median proteinuria (24 hour urine collection) at baseline was 1.89 g/d (IQR 1.09, 3.51 g/d). At week 29, the change in proteinuria was-1.00 g/d (IQR-1.82, −0.79 g/d), corresponding to a 52% reduction (FIG. 4). There was no change in eGFR at week 29 compared to baseline (mean±SD; BL 69±30; Wk29 70±24 mL/min/1.73 m2) (FIG. 5). The decreases of other complement components were similar in IgA Nephropathy patients to that observed in healthy subjects. The plasma level of the Factor B split product, Bb was decreased by 78%. Furthermore, the urinary level of Ba was reduced by 88% (FIG. 7). The serum alternative pathway activity, measured using a modified AH50 Wieslab alternative pathway ELISA assay (AH50 WAP), was decreased to −40%. The CH50 WAP activity was lowered only 8%. In addition, plasma CFBb was reduced approximately 69% and serum AH50 activity was decreased without observed changes in CH50.

Eight of the ten subjects administered Compound 696844 demonstrated reduced proteinuria as measured by 24-hour urine protein/creatinine ratio at the interim analysis. The 10 IgAN patients in the ongoing open label clinical Study for Compound 696844 were first maintained on maximal doses of ACE/ARB and then baseline 24-hr urine collections were obtained. The baseline UPCr level from the 24-hr collections of ranged from 1.00 to 3.58 g/g. The median UPCr at baseline was 1.49 g/g (Q1, Q3 of 1.12 and 1.90 g/g). The median reduction of proteinuria (UPCr from a 24-hr urine collection) was −45% when assessed on Week 29, following monthly dose of 70 mg SC. The range of change of proteinuria was from −87% to +26%. At Week 29, the median eGFR (estimated creatinine adjusted CKD-EPI 2021) was maintained (+4%) as compared to baseline (67.7 mL/1.73 m2).

Compound 696844 demonstrated a favorable safety profile with no treatment emergent severe adverse events and the only clinically meaningful safety signal (moderate treatment emergent adverse event (TEAE)) was an elevation of ALT (3-5 fold ULN), in one subject and a transient erythema at the injection site for 1 subject. All subjects completed the study (week 29).

### Example 3: Phase 2 Human Clinical Trial with Compound 696844 for Treatment of AMD-Associated Geographic Atrophy

A randomized, placebo-controlled, double-masked Phase 2 study in patients with geographic atrophy secondary to AMD was conducted. Subjects 50 years or older with study eye AMD-associated Geographic Atrophy, BCVA>35 letters, and GA area 1.9 mm² to ≤17 mm² without macular neovascularization have been enrolled. Two screening visits were performed three months apart to assess initial growth rate for randomization stratification. The median participant age is 76 and approximately 58% are female. Approximately 67% of GA lesions are subfoveal, 65% are multifocal, 95% are bilateral, and the baseline GA area is 7.6 mm². The annualized GA area change was approximately 2.0 mm². The growth rate was fastest for non-foveal centered multifocal lesions and slowest for unifocal non-foveal centered lesions. The BCVA was more than 2 lines better for multifocal lesions than unifocal lesions, and, as expected, was worse for foveal centered lesions.

Subjects were administered subcutaneous injections of Compound 696844, every four weeks, with a primary analysis 12 months after the start of treatment. In stage 1, subjects received 40 mg, 70 mg, or 100 mg of Compound 696844 from week 1 to week 45, or placebo matching solution (“placebo”); in stage 2, the 40 mg and 70 mg dosing cohorts were expanded in a new randomized group of participants based on the state 1 interim analysis at week 45. Subjects were assessed for primary outcome measures including absolute change from baseline in the Geographic Atrophy (GA) Area at week 49, as assessed by retinal imaging. Retinal imaging was assessed by fundus autofluorescence (FAF). Subjects were assessed for secondary outcome measures including percentage change from baseline in complement, including change levels of Factor B (CFB) in plasma (baseline and up to week 49); and percentage change from baseline in levels of serum AH50 activity (baseline and up to week 49); and absolute change from baseline in low luminance visual acuity (LLVA) (baseline and up to week 49).

Complement assays may include assays listed in Example 1, and may also include the following assessments: FB level (BECKMAN IMMAGE 800; nephelometry/turbidity); AH50 WAP (Weislab ELISA; deposition function ELISA); CH50 WCP (Weislab Elisa; deposition function ELISA); C3 level (BECKMAN IMMAGE 800; nephelometry/turbidity); Bb level (QUIDEL MICROVUE Bb PLUS EIA; ELISA Immunoassay); MBL level (BIOPORTO (distributed by QUIDEL); ELISA Immunoassay); sC5b-9 level (QUIDEL MICROVUE sC5b-9 PLUS EIA; ELISA Immunoassay); Factor D level (MILLIPORE Complement Panel #1; multiplex Luminex immunoassay); C2 level (MILLIPORE Complement Panel #1; Multiplex Luminex Immunoassay); and C4 level (BECKMAN IMMAGE 800; nephelometry/turbidity).

### Example 4: Phase 3 Human Clinical Trial with Compound 696844 for IgA Nephropathy

A randomized, double-blind, placebo-controlled Phase 3 clinical trial is conducted to evaluate the efficacy and safety of subcutaneously administered Compound 696844 in adult human subjects with IgA Nephropathy.

### Example 5: Phase 3 Human Clinical Trial with Compound 696844 for Geographic Atrophy Associated with AMD

A randomized, double-blind, placebo-controlled Phase 3 clinical trial is conducted to evaluate the efficacy and safety of subcutaneously administered Compound 696844 in adult human subjects with Geographic Atrophy associated with AMD.

1-214. (canceled)

215. A method of ameliorating IgA Nephropathy in a human subject, comprising administering to the human subject a dose comprising about 10 mg to about 100 mg of a compound according to the following chemical structure: 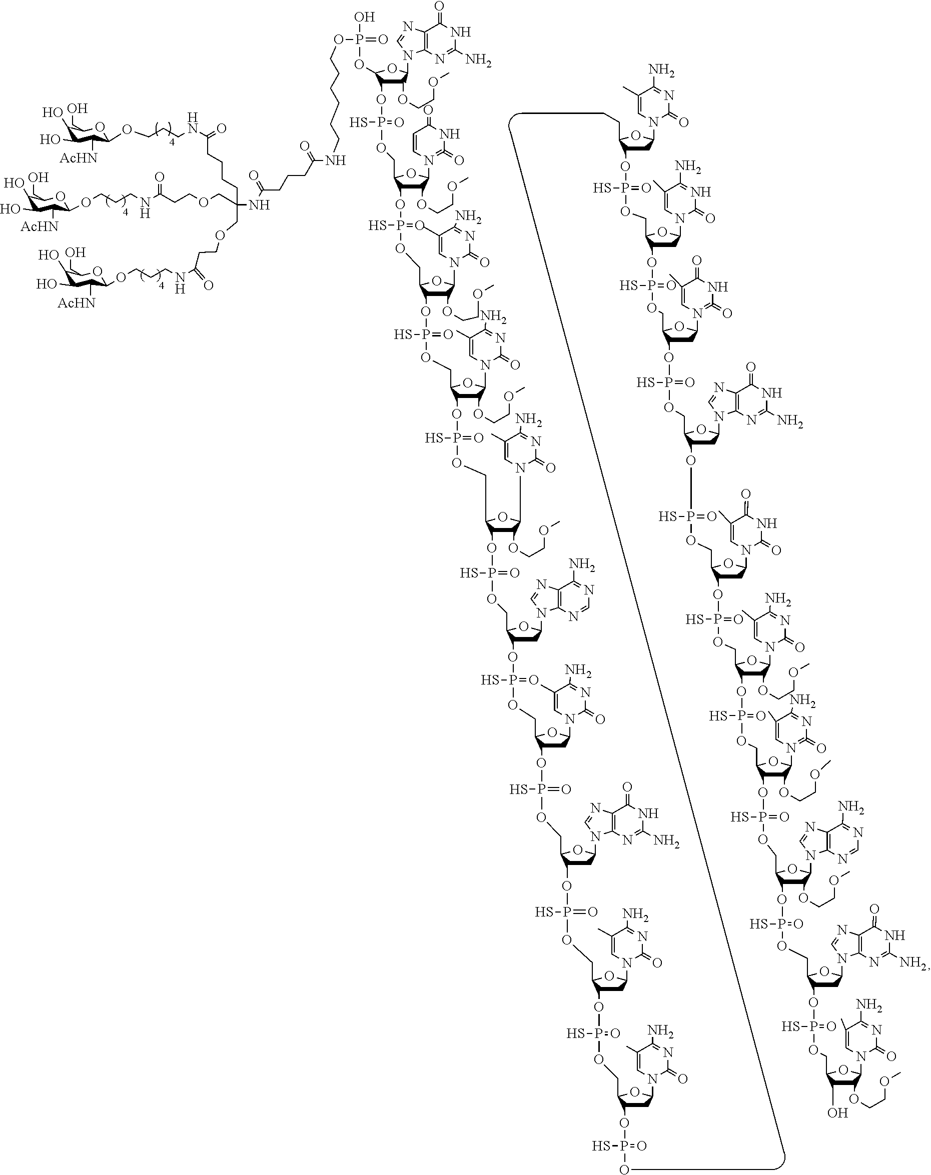
or a pharmaceutically acceptable salt thereof.

216. The method of claim 215, wherein the compound is the sodium salt or the potassium salt.

217. The method of claim 215, wherein the compound is the sodium salt of Compound 696844, represented by the following chemical structure:

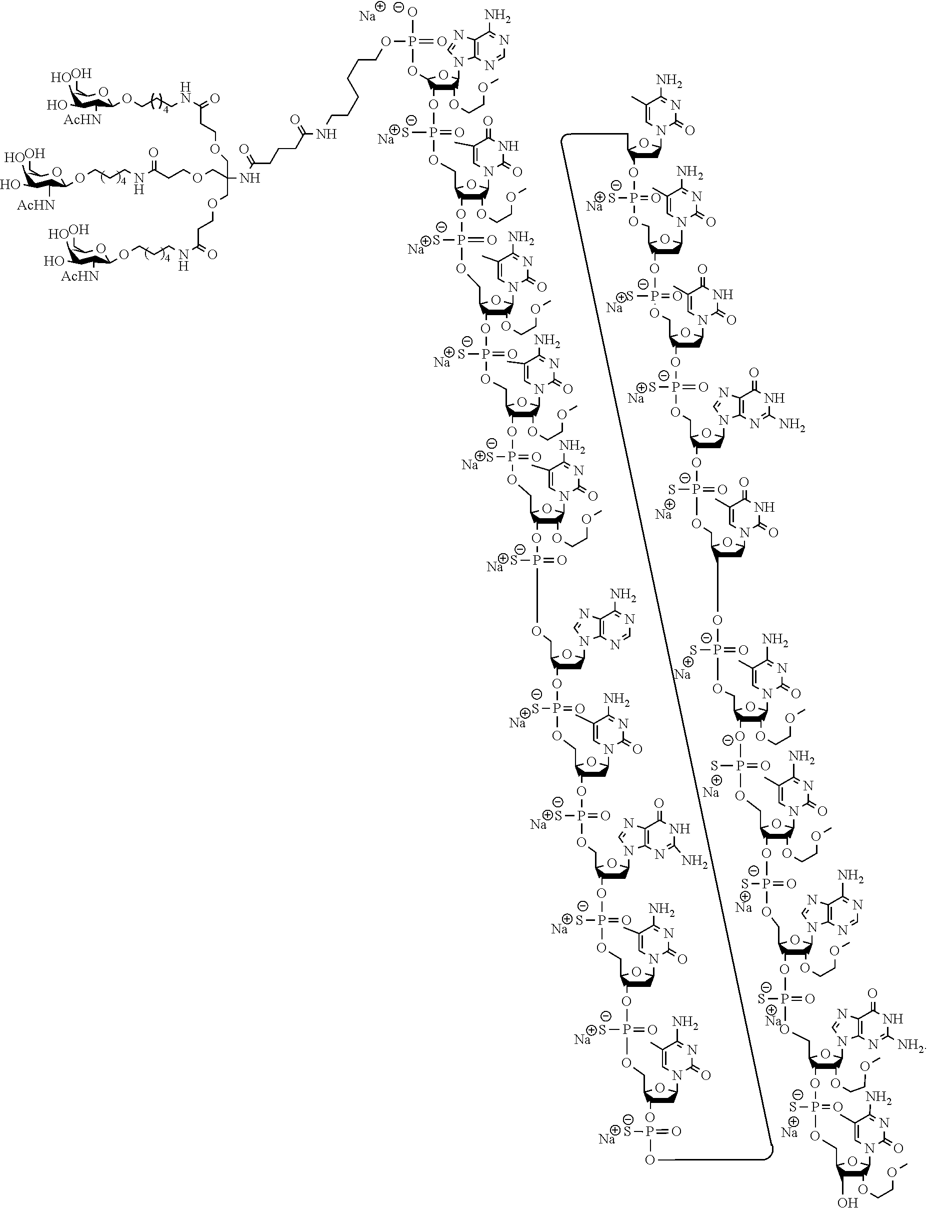

218. The method of claim 215, wherein the dose of the compound is about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg.

219. The method of claim 215, wherein the dose of the compound is about 70 mg, about 60 to about 70 mg, about 65 to about 75 mg, about 70 to about 75 mg, or about 70 to about 80 mg.

220. The method of claim 219, wherein the dose of the compound is equivalent to 70 mg of the fully protonated form of Compound 696844.

221. The method of claim 215, wherein the dose is administered once about every week, once about every two weeks, once about every 3 weeks, once about every 4 weeks, once about every 28 days, once about every month, once about every 8 weeks, once about every two months, once about every 12 weeks, once about every 16 weeks, or once about every 24 weeks.

222. The method of claim 217, wherein the dose of the compound is about 70 mg, about 60 to about 70 mg, about 65 to about 75 mg, about 70 to about 75 mg, or about 70 to about 80 mg.

223. The method of claim 222, wherein the dose of the compound is equivalent to 70 mg of the fully protonated form of Compound 696844.

224. The method of claim 217, wherein the dose is administered once about every week, once about every two weeks, once about every 3 weeks, once about every 4 weeks, once about every 28 days, once about every month, once about every 8 weeks, once about every two months, once about every 12 weeks, once about every 16 weeks, or once about every 24 weeks.

225. The method of claim 221, wherein the dose is administered once about every 4 weeks, once about every 28 days, or once about every month.

226. The method of claim 221, wherein the dose is administered at day 15 or at week 2, and at week 3.

227. The method of claim 215, comprising administering at least 2 doses, at least 3 doses, at least 4 doses, at least 5 doses, or at least 6 doses.

228. The method of claim 215, wherein the dose is administered on Day 1, Day 15, and Day 29.

229. The method of claim 215, wherein the dose is administered on Day 1, Day 15, and Day 29 and once every 4 weeks thereafter.

230. The method of claim 215 wherein the dose is administered:
(i) on Day 1, Day 15, and Day 29; and
(ii) once about every 4 weeks, once about every 28 days, or once about every month thereafter.

231. The method of claim 215, wherein administering the compound reduces the level of plasma CFB protein, reduces serum AP activity, reduces the level of serum functional CFB, reduces the level of urinary Factor B split product (Ba), or reduces the level of proteinuria in the subject.

232. The method of claim 215, wherein the administering comprises administration by injection selected from intravenous, subcutaneous, intramuscular, intrathecal (IT), and intracerebroventricular (ICV).

233. The method of claim 229, wherein the administration is subcutaneous administration.


SEQUENCE LISTING (6 sequences)

SEQ ID NO: 1 - RNA, 2646 nt, Homo sapiens
  source 1..2646: mol_type = mRNA
gacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccgatggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaacaaaaaaaaaaaaaaaaaaaa

SEQ ID NO: 2 - DNA, 9001 bp, Homo sapiens
  source 1..9001: mol_type = genomic DNA
ggaggtgagggtctcaggttggggatgctgggatccccctgtgacagctcccagaatgtctctcttccttctccaggtctggctgctttctctctctgacgcgggtcacccctcctcccaagcctcacaaacctgctaggtgtccctgggtctgcttattctttttttgttgttattgagatggagtcttgctctgtctcccaggctggagtgcagtggcacgacctcagctcactgcaacttctgcctcctgggttcaagcgattctcctacttcagcctcccgagtagctgagattacaggtgcccaccaccacaccagctaatttttgtatttttagtagagacgggatttcgccatgttggccaggatggtcttgaactcctgacctcaagtgatctgcctgcctcaacctcccaaagtgctgagattacaggcgtgagccactgcacccacccgggtctgcttattctacccttctctctggttccacccctgctgcagtggacaagctgtgccgaggttgtctcccaagaaaaaaccatgttccccaacttgacagatgtcagggaggtggtgacagaccagttcctatgcagtgggacccaggaggatgagagtccctgcaagggtgagtccctcaccatgcctggattcccaaggggaaggccacctgtgtctctgtggccagcatgcatgccagaacaccagtccactgccctagatgacactgtctcctgtcaccctttgctggcaggagaatctgggggagcagttttccttgagcggagattcaggttttttcaggtgagaaggtagaagcttgcaggacccaggggttacaggatctcagccttgttggggggatgagggaggcctttgagggatctagggaggttggggcttacagttggggctgtggcagcctcccagccagttctctccttttctccaggtgggtctggtgagctggggtctttacaacccctgccttggctctgctgacaaaaactcccgcaaaagggcccctcgtagcaaggtcccgccgccacgagactttcacatcaatctcttccgcatgcagccctggctgaggcagcacctgggggatgtcctgaattttttacccctctagccatggccactgagccctctgctgccctgccagaatctgccgcccctccatcttctacctctgaatggccacccttagaccctgtgatccatcctctctcctagctgagtaaatccgggtctctaggatgccagaggcagcgcacacaagctgggaaatcctcagggctcctaccagcaggactgcctcgctgccccacctcccgctccttggcctgtccccagattccttccctggttgacttgactcatgcttgtttcactttcacatggaatttcccagttatgaaattaataaaaatcaatggtttccacatctctcagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtaagcgagggtaaccttcccttcctgctgtctccagcatccctccttggccttttggggccaggcttcatcagcctttctcttcaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagaggtttgagggcaatgagtgtgggcagtggcctaaggcagaaacagggcaggcggcagcaaggtcaggactaggatgagactaggcagggtgacaaggtgggctgaccgggagtaggagcagttttagggtggcaggcggaaagggggcaagaaaaagcggagttaacccttactaagcatttaccctgggcttccaggcagccctggaagtcaagagaacactcagaaatggggagggagaagcagtggaaatccatatgggttgaggagtaggtaagatgctgcttctgcgggactgggaatgcgctgtttctcagtgacatggtctccgagaccaggagggatacacctaaggcagcctttccctcttgatgacttctacttgtccccccttctcaaagcaatccactgtccaagaccacacgacttcgagaacggggaatactggccccggtctccctactacaatgtgagtgatgagatctctttccactgctatgacggttacactctccggggctctgccaatcgcacctgccaagtgaatggccgatggagtgggcagacagcgatctgtgacaacggaggtgagaagcatcccctccccctacattgctgtctccctgacggcgcccagcccgaggagtgggcactcggctccggacactgtaactcttgctctctaccttgctcacggggcctcaggcttcagtgcttacctcgatgtctcatacctctgcagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaaggtgacctttgacctgtacccccaggtcagatcctggtcttccatcctactgtcttctctccccacctcaaccctgctctttcctcactttgtttaaacctccctgtacaactatctcacttctgagccttttataccctggaaacccatgatcccccgtctctttggtcactgtatccctgacactcccagacatttgacctcatttctgactctcccagactccttcatgtacgacacccctcaagaggtggccgaagctttcctgtcttccctgacagagaccatagaaggagtcgatgctgaggatgggcacggcccaggtttgaagacagagaagggaggcagggcagggaactgggggaaaatggagaagggacagaactgttaatgctggagcctgagccactctcctggcacccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggaatcctcctatccctgaactcgggggaatggaatctcgctgatcttccaggactagctccctgatcattccagcccctctgaacaacagggccccaggaaaatctccaggtcctattctgtcctccttcccttttacttgaagcagtttcttgactggtaattcctccatgaacctcagcccttgagcctcttactgagagcctccctgtcccagcaaagtcgctgaaatctcccaatcacagtattctattttcaatgccatggcgccttgttctcctcacccacaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaaggtcagaggttagggaatggtgggaggttcactttggggtcaggaggttcagggtggagggggtcatgagactaccttgagggcgacagggaggaccactttgtagtcaaaagttgaacagcaggatcgttgggcaatggaggttagtgggaacctgttgggggctggaagggccactttgtggtcaaagggaagtccgtgtaatgatgattaacttaaaaagttgaaagatgtgggatttcagttgcagattggtctctggggttaaaagatggcttggaagaccaggtgaggtgatggtctcttccctctccacagaccacaagttgaagtcagggactaacaccaagaaggccctccaggcagtgtacagcatgatgagctggccagatgacgtccctcctgaaggctggaaccgcacccgccatgtcatcatcctcatgactgatggtcagaagggacctctctcctgtcccagcctccccaccttctcagaccagcatgtggcccttaagtccacttgtaacactatacccatggttggggccctgaatgtgactcatagctggctgttcatctctcctgtgacccttcataaggaattcttcctaagccctgtgatcaactatctctaacccttcctcaacttgctcaccctgccatgtgtatccctgcctttagccagtttatcttccttatctcctaccctcatggtcctgtctcttctgcaggattgcacaacatgggcggggacccaattactgtcattgatgagatccgggacttgctatacattggcaaggatcgcaaaaacccaagggaggattatctgggtgagtaacctgcctaggacccagcaccccacttcctcagggcttggaccctcatccttcctttttatccctcagatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcggtagggagatacaagggaataaagaacacaactctcctcaggttcccctgaagtaattcattcttcctctacacctgaagctctagttgcctggaaagccttcttcattcctccttctctacctcagtgtcactattcttgtttcctggcactgttcacttaaccttagaatcacagagctctgagcacttcagagatctttctatagtcctacatttgacacgtggaaacagaagccaaaggaggtcaagggacagcaagttagcaacaagggtgggcttgaaaacagccaggcctctgacagcttgatcccaagttctttcccttttcagtccaccatagcagttttctcctaacacgaggaaacaaatacccgtggtctttccctttctccttttgggcctttgctccccatagactcctacccaaaaggctgctgccatttgggaatgaagtgttccgagttttcagcacattctccttctctgccagatgaaagccagtctctgagtctctgtggcatggtttgggaacacaggaagggtaccgattaccacaagcaaccatggcaggccaagatctcagtcattgtaagcacagaatcccagtagtggggacttgggggaggtgaggtcaaggtgaaatgggagtaggggaaggaaaaaatggccataagagatggtggtttgtgaaagttgagctttccctctctactgttgtgtccccagcgcccttcaaagggacacgagagctgtatgggggctgtggtgtctgagtactttgtgctgacagcagcacattgtttcactgtggatgacaaggaacactcaatcaaggtcagcgtaggtaaggatgcaactgaaggtcctgggctgcacctatgctctccaggcaacacctcccactttctacagatcctacactccacccatcctcaatgcagccccattccttgcaccccagaccagtcagggatgggggaagacgtgaagttaggaatgacacggggccagaggcaggaagctgcccacaaagaggtggtacctactctcctacttcaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggtgagagcgtccagatccctgaggaaaggctgggaaaggctggaggactggggtgaggagcaggcctggtttgctgttctccttgtcctttataggcccatttgtctcccctgcaccgagggaacaactcgagctttgaggcttcctccaactaccacttgccagcaacaaagtaagacatacttggcaagaggataaggatgagatcccaagagacaagtggggcatgagagggaggtgcaataggaagagatgatgcctggcccagaacctagctctagaagggcttaggggacatctactgagtgacaaaggcaatggggagatgacagtggtgggagcagctgaagtgacgcagtctattcgtccagaggaagagctgctccctgcacaggatatcaaagctctgtttgtgtctgaggaggagaaaaagctgactcggaaggaggtctacatcaagaatggggataaggtgagaaacgggcatcctaaggaggcactctaggccccaatccttcctaagccacttctgttcattacttctccatgcttcccacctcccctacagaaaggcagctgtgagagagatgctcaatatgccccaggctatgacaaagtcaaggacatctcagaggtggtcacccctcggttcctttgtactggaggagtgagtccctatgctgaccccaatacttgcagaggtgagagaatgctctttggttgtgctacaagtgcccaaggcccaacagtccttttctctacagcttctcctctccttgcaggtgattctggcggccccttgatagttcacaagagaagtcgtttcattcaagtgagtcctccctttcctatctggggagatgccaagtggtcagcatgggccccaaagcaggaaagctcaatgcatgtggctagtaattcgaggtaggcagagcctgcctcaccttaggaccgcatgtcttgcctgcgtgtgtcaagaacgaggctgagctgggtccctagtctgattcctttaggtcagctaagacacaagcaggaacagccatgcttccaggattaggaattctactgaatgatccatggcaccccactgcctctgcaggttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaacacctgtgttccagatccttttggggcaagggagtggggaacaggcactggccatgttgttacactgagatcaaacctgacagccgtttttaaaggtttaaccccaatcccaagtgctgaaaaaccagaggctgagggagatgtgtaagcttccacctcagtgttttactgagaccagcattggggcatatgaggcacaaggaatccagctctgttccctagaagccatccacaaggttttccttgtagacgtcatcactgtagacaatctgggtcctcttgtcccggtggcaacccttagggctgttctggacagctagggagggaggagaggaacagttaaggtctaaaggagatcatagaacagaccctgaggctgactcctgaccacctcactcctggccactggcccctggaagcccagtttccacgctgccctctggtggccaggatggcctgtcttccttagctcctttgtgccaacccatggccaagaaaagtataagtggacattttgatgaatgttttgttcttagaaaaatcccaaatgtcattgttgagacacgtgaatgatattaacccactacttacagtcagtatgtcagaagctaaaaactagaaaacctctgtagcccttttttgacatgctggtcaattctagttcctttcttttgcctgaagggccactgtagctgagcccttctttctgctcactcctttcccaggaaaatctactttcagggaaaatggattattcacactaagaaatgctactagctccaccagaactcattcagggtgtagctttggccctcaccattctctctcaagcctctagctgtttcttccccttcctctttcctccctccaccagacatgttactctcttcaccccatccaatggttccatccccaccacccttgagctacagagaatctctctcacccactcccatcctgtgatctctgtgcctcaacactgctggctactccctctttctcaaagtgtgtgtccttttgcttcagtggcccaggcccctgcggtgctgctcccagccctccgacccctcctcctgtctcctttgctaacgttaggctcaacgttagcctaacatgtcaggacagctggggacatgtgggg

SEQ ID NO: 3 - DNA, 20 bp, synthetic construct
  source 1..20: mol_type = other DNA
atcccacgcccctgtccagc

SEQ ID NO: 4 - DNA, 20 bp, synthetic construct
  source 1..20: mol_type = other DNA
  modified_base <1: mod_base = OTHER; note = THA-GalNAc phosphodiester
  modified_base 1: mod_base = OTHER; note = 2'-MOE adenosine 3'-phosphorothioate
  modified_base 2: mod_base = OTHER; note = 2'-MOE thymidine 3'-phosphorothioate
  modified_base 3..5: mod_base = OTHER; note = 2'-MOE 5-methylcytidine 3'-phosphorothioate
  modified_base 6: mod_base = OTHER; note = 2'-deoxyadenosine 3'-phosphorothioate
  modified_base 7: mod_base = OTHER; note = 5-methyl 2'-deoxycytidine 3'-phosphorothioate
  modified_base 8: mod_base = OTHER; note = 2'-deoxyguanosine 3'-phosphorothioate
  modified_base 9..12: mod_base = OTHER; note = 5-methyl 2'-deoxycytidine 3'-phosphorothioate
  modified_base 13: mod_base = OTHER; note = 2'-deoxythymidine 3'-phosphorothioate
  modified_base 14: mod_base = OTHER; note = 2'-deoxyguanosine 3'-phosphorothioate
  modified_base 15: mod_base = OTHER; note = 2'-deoxythymidine 3'-phosphorothioate
  modified_base 16..17: mod_base = OTHER; note = 2'-MOE 5-methylcytidine 3'-phosphorothioate
  modified_base 18: mod_base = OTHER; note = 2'-MOE adenosine 3'-phosphorothioate
  modified_base 19: mod_base = OTHER; note = 2'-MOE guanosine 3'-phosphorothioate
  modified_base 20: mod_base = OTHER; note = 2'-MOE 5-methylcytidine
atcccacgcccctgtccagc

SEQ ID NO: 5 - DNA, 12000 bp, Homo sapiens
  source 1..12000: mol_type = genomic DNA
gagaactgaatgagctagggtccaagaaggatggtgagaggcatgccttcattctgcaggacacaaaggctctgcaccaggtctttgaacatatgctgggtgagtgagctttgccctccttggtgtggggaggatggtgaggagcccgccagaggcccgtgttgggaacctggacacagtgcccctcacttgcctccttccccatctgatcctcacacccacagatgtctccaagctcacagacaccatctgcggggtggggaacatgtcagcaaacgcctctgaccaggagaggacaccctggcatgtcactattaaggtaccaggaaggaggggcagggcttggattccagaggtaaaagcggccatgggccagacatactgcaatctctgaaaatcacctgttcccctgcagcccaagagccaagagacctgccggggggccctcatctccgaccaatgggtcctgacagcagctcattgcttccgcgatggcaacgaccactccctgtggagggtcaatgtgggtaaggcaggggatgcaccagcctcctgatcctgaagccacagatcctaccacctcacccagcctctggcccctgcaggagccctggtctagcctaatctagtgtatcatttccaggagaccccaaatcccagtggggcaaagaattccttattgagaaggcggtgatctccccagggtttgatgtctttgccaaaaagaaccagggaatcctggagttctatggtgatgacatagctctgctgaagctggcccagaaagtaaagatgtccacccatgccaggtgcctggagtctgggatgggagggtgccctgcagggaagagtgctctggagatccctggaagagatactggggacaggctggtgtgacccttgctcttctccccaggcccatctgccttccctgcacgatggaggccaatctggctctgcggagacctcaaggcagcacctgtagggaccatggtgagtgctgggacttatggtgcttgagagctggggccggggtttgggggtgataacaaggactaggctgcagtccccaagccaggaacctggattctgggtaaaaggaccagcaccaacatccccttctcttgactatagagaatgaactgctgaacaaacagagtgttcctgctcattttgtcgccttgaatgggagcaaactgaacattaaccttaagatgggagtggaggtgagggtctcaggttggggatgctgggatccccctgtgacagctcccagaatgtctctcttccttctccaggtctggctgctttctctctctgacgcgggtcacccctcctcccaagcctcacaaacctgctaggtgtccctgggtctgcttattctttttttgttgttattgagatggagtcttgctctgtctcccaggctggagtgcagtggcacgacctcagctcactgcaacttctgcctcctgggttcaagcgattctcctacttcagcctcccgagtagctgagattacaggtgcccaccaccacaccagctaatttttgtatttttagtagagacgggatttcgccatgttggccaggatggtcttgaactcctgacctcaagtgatctgcctgcctcaacctcccaaagtgctgagattacaggcgtgagccactgcacccacccgggtctgcttattctacccttctctctggttccacccctgctgcagtggacaagctgtgccgaggttgtctcccaagaaaaaaccatgttccccaacttgacagatgtcagggaggtggtgacagaccagttcctatgcagtgggacccaggaggatgagagtccctgcaagggtgagtccctcaccatgcctggattcccaaggggaaggccacctgtgtctctgtggccagcatgcatgccagaacaccagtccactgccctagatgacactgtctcctgtcaccctttgctggcaggagaatctgggggagcagttttccttgagcggagattcaggttttttcaggtgagaaggtagaagcttgcaggacccaggggttacaggatctcagccttgttggggggatgagggaggcctttgagggatctagggaggttggggcttacagttggggctgtggcagcctcccagccagttctctccttttctccaggtgggtctggtgagctggggtctttacaacccctgccttggctctgctgacaaaaactcccgcaaaagggcccctcgtagcaaggtcccgccgccacgagactttcacatcaatctcttccgcatgcagccctggctgaggcagcacctgggggatgtcctgaattttttacccctctagccatggccactgagccctctgctgccctgccagaatctgccgcccctccatcttctacctctgaatggccacccttagaccctgtgatccatcctctctcctagctgagtaaatccgggtctctaggatgccagaggcagcgcacacaagctgggaaatcctcagggctcctaccagcaggactgcctcgctgccccacctcccgctccttggcctgtccccagattccttccctggttgacttgactcatgcttgtttcactttcacatggaatttcccagttatgaaattaataaaaatcaatggtttccacatctctcagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtaagcgagggtaaccttcccttcctgctgtctccagcatccctccttggccttttggggccaggcttcatcagcctttctcttcaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagaggtttgagggcaatgagtgtgggcagtggcctaaggcagaaacagggcaggcggcagcaaggtcaggactaggatgagactaggcagggtgacaaggtgggctgaccgggagtaggagcagttttagggtggcaggcggaaagggggcaagaaaaagcggagttaacccttactaagcatttaccctgggcttccaggcagccctggaagtcaagagaacactcagaaatggggagggagaagcagtggaaatccatatgggttgaggagtaggtaagatgctgcttctgcgggactgggaatgcgctgtttctcagtgacatggtctccgagaccaggagggatacacctaaggcagcctttccctcttgatgacttctacttgtccccccttctcaaagcaatccactgtccaagaccacacgacttcgagaacggggaatactggccccggtctccctactacaatgtgagtgatgagatctctttccactgctatgacggttacactctccggggctctgccaatcgcacctgccaagtgaatggccgatggagtgggcagacagcgatctgtgacaacggaggtgagaagcatcccctccccctacattgctgtctccctgacggcgcccagcccgaggagtgggcactcggctccggacactgtaactcttgctctctaccttgctcacggggcctcaggcttcagtgcttacctcgatgtctcatacctctgcagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaaggtgacctttgacctgtacccccaggtcagatcctggtcttccatcctactgtcttctctccccacctcaaccctgctctttcctcactttgtttaaacctccctgtacaactatctcacttctgagccttttataccctggaaacccatgatcccccgtctctttggtcactgtatccctgacactcccagacatttgacctcatttctgactctcccagactccttcatgtacgacacccctcaagaggtggccgaagctttcctgtcttccctgacagagaccatagaaggagtcgatgctgaggatgggcacggcccaggtttgaagacagagaagggaggcagggcagggaactgggggaaaatggagaagggacagaactgttaatgctggagcctgagccactctcctggcacccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggaatcctcctatccctgaactcgggggaatggaatctcgctgatcttccaggactagctccctgatcattccagcccctctgaacaacagggccccaggaaaatctccaggtcctattctgtcctccttcccttttacttgaagcagtttcttgactggtaattcctccatgaacctcagcccttgagcctcttactgagagcctccctgtcccagcaaagtcgctgaaatctcccaatcacagtattctattttcaatgccatggcgccttgttctcctcacccacaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaaggtcagaggttagggaatggtgggaggttcactttggggtcaggaggttcagggtggagggggtcatgagactaccttgagggcgacagggaggaccactttgtagtcaaaagttgaacagcaggatcgttgggcaatggaggttagtgggaacctgttgggggctggaagggccactttgtggtcaaagggaagtccgtgtaatgatgattaacttaaaaagttgaaagatgtgggatttcagttgcagattggtctctggggttaaaagatggcttggaagaccaggtgaggtgatggtctcttccctctccacagaccacaagttgaagtcagggactaacaccaagaaggccctccaggcagtgtacagcatgatgagctggccagatgacgtccctcctgaaggctggaaccgcacccgccatgtcatcatcctcatgactgatggtcagaagggacctctctcctgtcccagcctccccaccttctcagaccagcatgtggcccttaagtccacttgtaacactatacccatggttggggccctgaatgtgactcatagctggctgttcatctctcctgtgacccttcataaggaattcttcctaagccctgtgatcaactatctctaacccttcctcaacttgctcaccctgccatgtgtatccctgcctttagccagtttatcttccttatctcctaccctcatggtcctgtctcttctgcaggattgcacaacatgggcggggacccaattactgtcattgatgagatccgggacttgctatacattggcaaggatcgcaaaaacccaagggaggattatctgggtgagtaacctgcctaggacccagcaccccacttcctcagggcttggaccctcatccttcctttttatccctcagatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcggtagggagatacaagggaataaagaacacaactctcctcaggttcccctgaagtaattcattcttcctctacacctgaagctctagttgcctggaaagccttcttcattcctccttctctacctcagtgtcactattcttgtttcctggcactgttcacttaaccttagaatcacagagctctgagcacttcagagatctttctatagtcctacatttgacacgtggaaacagaagccaaaggaggtcaagggacagcaagttagcaacaagggtgggcttgaaaacagccaggcctctgacagcttgatcccaagttctttcccttttcagtccaccatagcagttttctcctaacacgaggaaacaaatacccgtggtctttccctttctccttttgggcctttgctccccatagactcctacccaaaaggctgctgccatttgggaatgaagtgttccgagttttcagcacattctccttctctgccagatgaaagccagtctctgagtctctgtggcatggtttgggaacacaggaagggtaccgattaccacaagcaaccatggcaggccaagatctcagtcattgtaagcacagaatcccagtagtggggacttgggggaggtgaggtcaaggtgaaatgggagtaggggaaggaaaaaatggccataagagatggtggtttgtgaaagttgagctttccctctctactgttgtgtccccagcgcccttcaaagggacacgagagctgtatgggggctgtggtgtctgagtactttgtgctgacagcagcacattgtttcactgtggatgacaaggaacactcaatcaaggtcagcgtaggtaaggatgcaactgaaggtcctgggctgcacctatgctctccaggcaacacctcccactttctacagatcctacactccacccatcctcaatgcagccccattccttgcaccccagaccagtcagggatgggggaagacgtgaagttaggaatgacacggggccagaggcaggaagctgcccacaaagaggtggtacctactctcctacttcaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggtgagagcgtccagatccctgaggaaaggctgggaaaggctggaggactggggtgaggagcaggcctggtttgctgttctccttgtcctttataggcccatttgtctcccctgcaccgagggaacaactcgagctttgaggcttcctccaactaccacttgccagcaacaaagtaagacatacttggcaagaggataaggatgagatcccaagagacaagtggggcatgagagggaggtgcaataggaagagatgatgcctggcccagaacctagctctagaagggcttaggggacatctactgagtgacaaaggcaatggggagatgacagtggtgggagcagctgaagtgacgcagtctattcgtccagaggaagagctgctccctgcacaggatatcaaagctctgtttgtgtctgaggaggagaaaaagctgactcggaaggaggtctacatcaagaatggggataaggtgagaaacgggcatcctaaggaggcactctaggccccaatccttcctaagccacttctgttcattacttctccatgcttcccacctcccctacagaaaggcagctgtgagagagatgctcaatatgccccaggctatgacaaagtcaaggacatctcagaggtggtcacccctcggttcctttgtactggaggagtgagtccctatgctgaccccaatacttgcagaggtgagagaatgctctttggttgtgctacaagtgcccaaggcccaacagtccttttctctacagcttctcctctccttgcaggtgattctggcggccccttgatagttcacaagagaagtcgtttcattcaagtgagtcctccctttcctatctggggagatgccaagtggtcagcatgggccccaaagcaggaaagctcaatgcatgtggctagtaattcgaggtaggcagagcctgcctcaccttaggaccgcatgtcttgcctgcgtgtgtcaagaacgaggctgagctgggtccctagtctgattcctttaggtcagctaagacacaagcaggaacagccatgcttccaggattaggaattctactgaatgatccatggcaccccactgcctctgcaggttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaacacctgtgttccagatccttttggggcaagggagtggggaacaggcactggccatgttgttacactgagatcaaacctgacagccgtttttaaaggtttaaccccaatcccaagtgctgaaaaaccagaggctgagggagatgtgtaagcttccacctcagtgttttactgagaccagcattggggcatatgaggcacaaggaatccagctctgttccctagaagccatccacaaggttttccttgtagacgtcatcactgtagacaatctgggtcctcttgtcccggtggcaacccttagggctgttctggacagctagggagggaggagaggaacagttaaggtctaaaggagatcatagaacagaccctgaggctgactcctgaccacctcactcctggccactggcccctggaagcccagtttccacgctgccctctggtggccaggatggcctgtcttccttagctcctttgtgccaacccatggccaagaaaagtataagtggacattttgatgaatgttttgttcttagaaaaatcccaaatgtcattgttgagacacgtgaatgatattaacccactacttacagtcagtatgtcagaagctaaaaactagaaaacctctgtagcccttttttgacatgctggtcaattctagttcctttcttttgcctgaagggccactgtagctgagcccttctttctgctcactcctttcccaggaaaatctactttcagggaaaatggattattcacactaagaaatgctactagctccaccagaactcattcagggtgtagctttggccctcaccattctctctcaagcctctagctgtttcttccccttcctctttcctccctccaccagacatgttactctcttcaccccatccaatggttccatccccaccacccttgagctacagagaatctctctcacccactcccatcctgtgatctctgtgcctcaacactgctggctactccctctttctcaaagtgtgtgtccttttgcttcagtggcccaggcccctgcggtgctgctcccagccctccgacccctcctcctgtctcctttgctaacgttaggctcaacgttagcctaacatgtcaggacagctggggacatgtggggtgtgaggtgaacagtcctgtttcctaacatagtcccagagtactcctcaaactgagtcctgggtcgtttttttttctctgaaatcagagtctccctgatgatcctattgtttggcagccaccctgtgatgtggatgacttaatctatgttttccttccttacctcacacctgagttccagatccctgatttcgaatacttatgaaactcactctactccatctcaaaatgaacaagccccatgagacactcatcttcctcaccaatctcactccagctcccactttcttccctgttccagtcactgctttggaagctgttttcaatccttttctctcctttctttacctctaactgacagaggatctgaaattttccttcccattcccatagcctccgcacacactctgacctcgatcatctctaggaaacccaaggatgtgtgggggaaccaaaaggaatggcctgtgggggagaggatgggaaaggaagaatcccattcttaccgagggagccccagacagacttgccagtagcggcatccagcatgggctgttttcgggctatgttgactttgagctgtacagactccacctgggtcccgttgagctgaagcagaagaggggaggcagaggatggggaggaaaacattacagataaaccaaagaagttattccaggagttgctatcctaggaggagactgaataaggaatctgagaatgtgagtttttctgtgtgaatagggagaggctttctttatcaagaggaaccaacttcttcctggcatctagtattttgaggagaacacatgagaacagcagaagcgatgggaagaacagatttgggaagttccaacctcagcaacggcctgatctgctgactccatcttttcataggtgacgaaggcacagctgggataagagaaaacacggtcagtggagagccaaggggctcttctggacccaaccaaacccagtgataataggcggctgcagggagggcagcttcttccctcaggtctcacaccccaggattctcccaggacttctcatcatgccctgttgtcatccttactttctgggtgggtccatggagaggtcaatgatgtttccaaaaggagagaaggccccacggagaagggtgggtgtcatgtcttctccatatacatagagagtattccctttcctaggggctcgccgttcagggaatgaatccgaccctttgggagcacaaatcatagtcacaagacatagaccatgccacatttcacttagtaggacccacataaacctcagttaaggtcaccttgacctccagccaaaatcactcactgcggaaaggaccctctcggtctcggtctcgatcccgctcccgatccctgtcccgttcccggtctcgatctcgatcccgatcccgatccctgtcccgctctctgtctctgtctcgatcccggtctcgatcccgctcccgatctcggtctctgtcccggttcctctcatggctgcggtcccggctgcggcttcggggaggggaggctgaggagtgggcaccactgcgttcttcatagccccagtcaaagcttcgagggggaccatcaccagcccctgggccctctgcctcttctccatctggtcctagttctcgaagtcgatcactagaagacacaaagctggggagatgcagactgaagatcaaaggggggttttaccttctcccctcagaccctgtggagactcaatattccctctatagcccagctcctacagcccaaacctcc

SEQ ID NO: 6 - DNA, 7637 bp, Homo sapiens
  source 1..7637: mol_type = genomic DNA
aggacccaggggttacaggatctcagccttgttggggggatgagggaggcctttgagggatctagggaggttggggcttacagttggggctgtggcagcctcccagccagttctctccttttctccaggtgggtctggtgagctggggtctttacaacccctgccttggctctgctgacaaaaactcccgcaaaagggcccctcgtagcaaggtcccgccgccacgagactttcacatcaatctcttccgcatgcagccctggctgaggcagcacctgggggatgtcctgaattttttacccctctagccatggccactgagccctctgctgccctgccagaatctgccgcccctccatcttctacctctgaatggccacccttagaccctgtgatccatcctctctcctagctgagtaaatccgggtctctaggatgccagaggcagcgcacacaagctgggaaatcctcagggctcctaccagcaggactgcctcgctgccccacctcccgctccttggcctgtccccagattccttccctggttgacttgactcatgcttgtttcactttcacatggaatttcccagttatgaaattaataaaaatcaatggtttccacatctctcagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtaagcgagggtaaccttcccttcctgctgtctccagcatccctccttggccttttggggccaggcttcatcagcctttctcttcaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagaggtttgagggcaatgagtgtgggcagtggcctaaggcagaaacagggcaggcggcagcaaggtcaggactaggatgagactaggcagggtgacaaggtgggctgaccgggagtaggagcagttttagggtggcaggcggaaagggggcaagaaaaagcggagttaacccttactaagcatttaccctgggcttccaggcagccctggaagtcaagagaacactcagaaatggggagggagaagcagtggaaatccatatgggttgaggagtaggtaagatgctgcttctgcgggactgggaatgcgctgtttctcagtgacatggtctccgagaccaggagggatacacctaaggcagcctttccctcttgatgacttctacttgtccccccttctcaaagcaatccactgtccaagaccacacgacttcgagaacggggaatactggccccggtctccctactacaatgtgagtgatgagatctctttccactgctatgacggttacactctccggggctctgccaatcgcacctgccaagtgaatggccgatggagtgggcagacagcgatctgtgacaacggaggtgagaagcatcccctccccctacattgctgtctccctgacggcgcccagcccgaggagtgggcactcggctccggacactgtaactcttgctctctaccttgctcacggggcctcaggcttcagtgcttacctcgatgtctcatacctctgcagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaaggtgacctttgacctgtacccccaggtcagatcctggtcttccatcctactgtcttctctccccacctcaaccctgctctttcctcactttgtttaaacctccctgtacaactatctcacttctgagccttttataccctggaaacccatgatcccccgtctctttggtcactgtatccctgacactcccagacatttgacctcatttctgactctcccagactccttcatgtacgacacccctcaagaggtggccgaagctttcctgtcttccctgacagagaccatagaaggagtcgatgctgaggatgggcacggcccaggtttgaagacagagaagggaggcagggcagggaactgggggaaaatggagaagggacagaactgttaatgctggagcctgagccactctcctggcacccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggaatcctcctatccctgaactcgggggaatggaatctcgctgatcttccaggactagctccctgatcattccagcccctctgaacaacagggccccaggaaaatctccaggtcctattctgtcctccttcccttttacttgaagcagtttcttgactggtaattcctccatgaacctcagcccttgagcctcttactgagagcctccctgtcccagcaaagtcgctgaaatctcccaatcacagtattctattttcaatgccatggcgccttgttctcctcacccacaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaaggtcagaggttagggaatggtgggaggttcactttggggtcaggaggttcagggtggagggggtcatgagactaccttgagggcgacagggaggaccactttgtagtcaaaagttgaacagcaggatcgttgggcaatggaggttagtgggaacctgttgggggctggaagggccactttgtggtcaaagggaagtccgtgtaatgatgattaacttaaaaagttgaaagatgtgggatttcagttgcagattggtctctggggttaaaagatggcttggaagaccaggtgaggtgatggtctcttccctctccacagaccacaagttgaagtcagggactaacaccaagaaggccctccaggcagtgtacagcatgatgagctggccagatgacgtccctcctgaaggctggaaccgcacccgccatgtcatcatcctcatgactgatggtcagaagggacctctctcctgtcccagcctccccaccttctcagaccagcatgtggcccttaagtccacttgtaacactatacccatggttggggccctgaatgtgactcatagctggctgttcatctctcctgtgacccttcataaggaattcttcctaagccctgtgatcaactatctctaacccttcctcaacttgctcaccctgccatgtgtatccctgcctttagccagtttatcttccttatctcctaccctcatggtcctgtctcttctgcaggattgcacaacatgggcggggacccaattactgtcattgatgagatccgggacttgctatacattggcaaggatcgcaaaaacccaagggaggattatctgggtgagtaacctgcctaggacccagcaccccacttcctcagggcttggaccctcatccttcctttttatccctcagatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcggtagggagatacaagggaataaagaacacaactctcctcaggttcccctgaagtaattcattcttcctctacacctgaagctctagttgcctggaaagccttcttcattcctccttctctacctcagtgtcactattcttgtttcctggcactgttcacttaaccttagaatcacagagctctgagcacttcagagatctttctatagtcctacatttgacacgtggaaacagaagccaaaggaggtcaagggacagcaagttagcaacaagggtgggcttgaaaacagccaggcctctgacagcttgatcccaagttctttcccttttcagtccaccatagcagttttctcctaacacgaggaaacaaatacccgtggtctttccctttctccttttgggcctttgctccccatagactcctacccaaaaggctgctgccatttgggaatgaagtgttccgagttttcagcacattctccttctctgccagatgaaagccagtctctgagtctctgtggcatggtttgggaacacaggaagggtaccgattaccacaagcaaccatggcaggccaagatctcagtcattgtaagcacagaatcccagtagtggggacttgggggaggtgaggtcaaggtgaaatgggagtaggggaaggaaaaaatggccataagagatggtggtttgtgaaagttgagctttccctctctactgttgtgtccccagcgcccttcaaagggacacgagagctgtatgggggctgtggtgtctgagtactttgtgctgacagcagcacattgtttcactgtggatgacaaggaacactcaatcaaggtcagcgtaggtaaggatgcaactgaaggtcctgggctgcacctatgctctccaggcaacacctcccactttctacagatcctacactccacccatcctcaatgcagccccattccttgcaccccagaccagtcagggatgggggaagacgtgaagttaggaatgacacggggccagaggcaggaagctgcccacaaagaggtggtacctactctcctacttcaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggtgagagcgtccagatccctgaggaaaggctgggaaaggctggaggactggggtgaggagcaggcctggtttgctgttctccttgtcctttataggcccatttgtctcccctgcaccgagggaacaactcgagctttgaggcttcctccaactaccacttgccagcaacaaagtaagacatacttggcaagaggataaggatgagatcccaagagacaagtggggcatgagagggaggtgcaataggaagagatgatgcctggcccagaacctagctctagaagggcttaggggacatctactgagtgacaaaggcaatggggagatgacagtggtgggagcagctgaagtgacgcagtctattcgtccagaggaagagctgctccctgcacaggatatcaaagctctgtttgtgtctgaggaggagaaaaagctgactcggaaggaggtctacatcaagaatggggataaggtgagaaacgggcatcctaaggaggcactctaggccccaatccttcctaagccacttctgttcattacttctccatgcttcccacctcccctacagaaaggcagctgtgagagagatgctcaatatgccccaggctatgacaaagtcaaggacatctcagaggtggtcacccctcggttcctttgtactggaggagtgagtccctatgctgaccccaatacttgcagaggtgagagaatgctctttggttgtgctacaagtgcccaaggcccaacagtccttttctctacagcttctcctctccttgcaggtgattctggcggccccttgatagttcacaagagaagtcgtttcattcaagtgagtcctccctttcctatctggggagatgccaagtggtcagcatgggccccaaagcaggaaagctcaatgcatgtggctagtaattcgaggtaggcagagcctgcctcaccttaggaccgcatgtcttgcctgcgtgtgtcaagaacgaggctgagctgggtccctagtctgattcctttaggtcagctaagacacaagcaggaacagccatgcttccaggattaggaattctactgaatgatccatggcaccccactgcctctgcaggttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaacacctgtgttccagatccttttggggcaagggagtggggaacaggcactggccatgttgttacactgagatcaaacctgacagccgtttttaaaggtttaaccccaatcccaagtgctgaaaaaccagaggctgagggagatgtgtaagcttccacctcagtgttttactgagaccagcattggggcatatgaggcacaaggaatccagctctgttccctagaagccatccacaaggttttccttgtagacgtcatcactgtagacaatctgggtcctcttgtcccggtggcaacccttagggctgttctggacagctagggagggaggagaggaacagttaaggtctaaaggagatcatagaacagaccctgaggctgactcctgaccacctcactcctggccactggcccctggaagcccagtttccacgctgccctctggtggccaggatggcctgtcttccttagctcctttgtgccaacccatggccaagaaaagtataagtggacattttgatgaatgttttgttcttagaaaaatcccaaatgtcattgttgagacacgtgaatgatattaacccactacttacagtcagtatgtca